(19) United States
(12) Patent Application Publication
(10) Pub. No.: US 2012/0151629 A1
(43) Pub. Date: Jun. 14, 2012

(54) Nematode-Resistant Transgenic Plants

(75) Inventors: Bonnie C. McCaig, Durham, NC (US); Aaron Wiig, Chapel Hill, NC (US)

(73) Assignee: BASF Plant Science Company GmbH, Ludwigshafen (DE)

(21) Appl. No.: 13/390,519

(22) PCT Filed: Aug. 13, 2010
(86) PCT No.: PCT/EP10/61799

§ 371 Date: Feb. 15, 2012

Related U.S. Application Data
(60) Provisional application No. 61/236,624, filed on Aug. 25, 2009.

(51) Int. Cl. A01H 1/00; A01H 5/10; C12N 15/82; A01H 5/00

(52) U.S. Cl. 800/279; 800/298; 435/320.1; 800/290

(57) ABSTRACT

The invention provides nematode-resistant transgenic plants and seed that express polynucleotides encoding AP2/EREBP transcription factors, harpin-induced proteins, TINY-like transcription factors, annexins, laccases, isoflavone 7-O-methyltransferases, anthocyanidin 3-glucoside rhanmosyltransferases, hsr201-like, or AUX/IAA proteins. The invention also provides methods of producing transgenic plants with increased resistance to plant parasitic nematodes and expression vectors for use in such methods.

### FIELD OF THE INVENTION

The invention relates to enhancement of agricultural productivity through use of nematode-resistant transgenic plants and seeds, and methods of making such plants and seeds.

### BACKGROUND OF THE INVENTION

Nematodes are microscopic roundworms that feed on the roots, leaves and stems of more than 2,000 row crops, vegetables, fruits, and ornamental plants, causing an estimated $100 billion crop loss worldwide. A variety of parasitic nematode species infect crop plants, including root-knot nematodes (RKN), cyst- and lesion-forming nematodes. Root-knot nematodes, which are characterized by causing root gall formation at feeding sites, have a relatively broad host range and are therefore parasitic on a large number of crop species. The cyst- and lesion-forming nematode species have a more limited host range, but still cause considerable losses in susceptible crops.

Parasitic nematodes are present throughout the United States, with the greatest concentrations occurring in the warm, humid regions of the South and West and in sandy soils. Soybean cyst nematode (Heterodera glycines), the most serious pest of soybean plants, was first discovered in the United States in North Carolina in 1954. Some areas are so heavily infested by soybean cyst nematode (SCN) that soybean production is no longer economically possible without control measures. Although soybean is the major economic crop attacked by SCN, SCN parasitizes some fifty hosts in total, including field crops, vegetables, ornamentals, and weeds.

Signs of nematode damage include stunting and yellowing of leaves, and wilting of the plants during hot periods. Nematode infestation, however, can cause significant yield losses without any obvious above-ground disease symptoms. The primary causes of yield reduction are due to underground root damage. Roots infected by SCN are dwarfed or stunted. Nematode infestation also can decrease the number of nitrogen-fixing nodules on the roots, and may make the roots more susceptible to attacks by other soil-borne plant nematodes.

The nematode life cycle has three major stages: egg, juvenile, and adult. The life cycle varies between species of nematodes. The life cycle of SCN is similar to the life cycles of other plant parasitic nematodes. The SCN life cycle can usually be completed in 24 to 30 days under optimum conditions, whereas other species can take as long as a year, or longer, to complete the life cycle. When temperature and moisture levels become favorable in the spring, worm-shaped juveniles hatch from eggs in the soil. Only nematodes in the juvenile developmental stage are capable of infecting soybean roots.

After penetrating soybean roots, SCN juveniles move through the root until they contact vascular tissue, at which time they stop migrating and begin to feed. With a stylet, the nematode injects secretions that modify certain root cells and transform them into specialized feeding sites. The root cells are morphologically transformed into large multinucleate syncytia (or giant cells in the case of RKN), which are used as a source of nutrients for the nematodes. The actively feeding nematodes thus steal essential nutrients from the plant resulting in yield loss. As female nematodes feed, they swell and eventually become so large that their bodies break through the root tissue and are exposed on the surface of the root.

After a period of feeding, male SCN, which are not swollen as adult females, migrate out of the root into the soil and fertilize the enlarged adult females. The males then die, while the females remain attached to the root system and continue to feed. The eggs in the swollen females begin developing, initially in a mass or egg sac outside the body, and then later within the nematode body cavity. Eventually the entire adult female body cavity is filled with eggs, and the nematode dies. It is the egg-filled body of the dead female that is referred to as the cyst. Cysts eventually dislodge and are found free in the soil. The walls of the cyst become very tough, providing excellent protection for the approximately 200 to 400 eggs contained within. SCN eggs survive within the cyst until proper hatching conditions occur. Although many of the eggs may hatch within the first year, many also will survive within the protective cysts for several years.

A nematode can move through the soil only a few inches per year on its own power. However, nematode infestation can spread substantial distances in a variety of ways. Anything that can move infested soil is capable of spreading the infestation, including farm machinery, vehicles and tools, wind, water, animals, and farm workers. Seed sized particles of soil often contaminate harvested seed. Consequently, nematode infestation can be spread when contaminated seed from infested fields is planted in non-infested fields. There is even evidence that certain nematode species can be spread by birds. Only some of these causes can be prevented.

Traditional practices for managing nematode infestation include: maintaining proper soil nutrients and soil pH levels in nematode-infested land; controlling other plant diseases, as well as insect and weed pests; using sanitation practices such as plowing, planting, and cultivating of nematode-infested fields only after working non-infested fields; cleaning equipment thoroughly with high pressure water or steam after working in infested fields; not using seed grown on infested land for planting non-infested fields unless the seed has been properly cleaned; rotating infested fields and alternating host crops with non-host crops; using nematicides; and planting resistant plant varieties.

Methods have been proposed for the genetic transformation of plants in order to confer increased resistance to plant parasitic nematodes. For example, U.S. Pat. Nos. 5,589,622 and 5,824,876 are directed to the identification of plant genes expressed specifically in or adjacent to the feeding site of the plant after attachment by the nematode. A number of approaches involve transformation of plants with double-stranded RNA capable of inhibiting essential nematode genes. Other agricultural biotechnology approaches propose to over-express genes that encode proteins that are toxic to nematodes.

To date, no genetically modified plant comprising a transgene capable of conferring nematode resistance has been deregulated in any country. Accordingly, a need continues to exist to identify safe and effective compositions and methods for controlling plant parasitic nematodes using agricultural biotechnology.

### SUMMARY OF THE INVENTION

The present inventors have discovered that transgenic overexpression of certain plant polynucleotides can render plants resistant to parasitic nematodes. In particular, overexpression of a plant polynucleotide selected from the group consisting of: a) an AP2/EREBP transcription factor polynucleotide similar to SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, or SEQ ID NO:19; b) a harpin-induced polynucleotide similar to SEQ ID NO:21, SEQ ID NO:23, SEQ ID NO:25, SEQ ID NO:27, SEQ ID NO:29, SEQ ID NO:31, SEQ ID NO:33, SEQ ID NO:35, or SEQ ID NO:37; c) a TINY-like polynucleotide similar to SEQ ID NO:39, SEQ ID NO:41, SEQ ID NO:43, SEQ ID NO:45, or SEQ ID NO:47; d) an annexin polynucleotide similar to SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, or SEQ ID NO:77; e) a laccase polynucleotide similar to SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, or SEQ ID NO:103; f) a benzoyl transferase polynucleotide similar to SEQ ID NO:105 or SEQ ID NO:107; g) a rhamnosyltransferase polynucleotide similar to SEQ ID NO:109, SEQ ID NO:111, SEQ ID NO:113, or SEQ ID NO:115; h) an isoflavone-7-O-methyltransferase polynucleotide similar to SEQ ID NO:117, SEQ ID NO:119, SEQ ID NO:121, SEQ ID NO:123, or SEQ ID NO:125; and i) an AUX/IAA polynucleotide similar to SEQ ID NO:127, SEQ ID NO:129, SEQ ID NO:131, or SEQ ID NO:133. Accordingly, the present invention provides transgenic plants and seeds, and methods to overcome, or at least alleviate, nematode infestation of valuable agricultural crops.

In one embodiment, the invention provides a transgenic plant transformed with an expression vector comprising an isolated polynucleotide selected from the group consisting of: a) a polynucleotide encoding an AP2/EREBP transcription factor similar to SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, or SEQ ID NO:20; b) a polynucleotide encoding a harpin-induced protein similar to SEQ ID NO:22, SEQ ID NO:24, SEQ ID NO:26, SEQ ID NO:28, SEQ ID NO:30, SEQ ID NO:32, SEQ ID NO:34, SEQ ID NO:36, or SEQ ID NO:38; c) a polynucleotide encoding a TINY-like transcription factor similar to SEQ ID NO:40, SEQ ID NO:42, SEQ ID NO:44, SEQ ID NO:46, or SEQ ID NO:48; d) a polynucleotide encoding an annexin protein similar to SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, or SEQ ID NO:78; e) a polynucleotide encoding a laccase similar to SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, or SEQ ID NO:104; f) a polynucleotide encoding a benzoyl transferase similar to SEQ ID NO:106 or SEQ ID NO:108; g) a polynucleotide encoding a rhamnosyltransferase similar to SEQ ID NO:110, SEQ ID NO:112, SEQ ID NO:114, or SEQ ID NO:116; h) a polynucleotide encoding an isoflavone-7-O-methyltransferase similar to SEQ ID NO:118, SEQ ID NO:120, SEQ ID NO:122, SEQ ID NO:124, or SEQ ID NO:126; and i) a polynucleotide encoding an AUX/IAA protein similar to SEQ ID NO:128, SEQ ID NO:130, SEQ ID NO:132, or SEQ ID NO:134.

Another embodiment of the invention provides a seed produced by the transgenic plant described above. The seed is true breeding for a transgene comprising at least one polynucleotide selected from the group consisting of: a) a polynucleotide encoding an AP2/EREBP transcription factor similar to SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, or SEQ ID NO:20; b) a polynucleotide encoding a harpin-induced protein similar to SEQ ID NO:22, SEQ ID NO:24, SEQ ID NO:26, SEQ ID NO:28, SEQ ID NO:30, SEQ ID NO:32, SEQ ID NO:34, SEQ ID NO:36, or SEQ ID NO:38; c) a polynucleotide encoding a TINY-like transcription factor similar to SEQ ID NO:40, SEQ ID NO:42, SEQ ID NO:44, SEQ ID NO:46, or SEQ ID NO:48; d) a polynucleotide encoding an annexin protein similar to SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, or SEQ ID NO:78; e) a polynucleotide encoding a laccase similar to SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, or SEQ ID NO:104; f) a polynucleotide encoding a benzoyl transferase similar to SEQ ID NO:106 or SEQ ID NO:108; g) a polynucleotide encoding a rhamnosyltransferase similar to SEQ ID NO:110, SEQ ID NO:112, SEQ ID NO:114, or SEQ ID NO:116; h) a polynucleotide encoding an isoflavone-7-O-methyltransferase similar to SEQ ID NO:118, SEQ ID NO:120, SEQ ID NO:122, SEQ ID NO:124, or SEQ ID NO:126; and i) a polynucleotide encoding an AUX/IAA protein similar to SEQ ID NO:128, SEQ ID NO:130, SEQ ID NO:132, or SEQ ID NO:134, and expression of the transgene confers increased nematode resistance to the plant grown from the transgenic seed.

In another embodiment, the invention provides an expression vector comprising a promoter operably linked to a polynucleotide encoding at least one polynucleotide selected from the group consisting of: a) a polynucleotide encoding an AP2/EREBP transcription factor similar to SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, or SEQ ID NO:20; b) a polynucleotide encoding a harpin-induced protein similar to SEQ ID NO:22, SEQ ID NO:24, SEQ ID NO:26, SEQ ID NO:28, SEQ ID NO:30, SEQ ID NO:32, SEQ ID NO:34, SEQ ID NO:36, or SEQ ID NO:38; c) a polynucleotide encoding a TINY-like transcription factor similar to SEQ ID NO:40, SEQ ID NO:42, SEQ ID NO:44, SEQ ID NO:46, or SEQ ID NO:48; d) a polynucleotide encoding an annexin protein similar to SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, or SEQ ID NO:78; e) a polynucleotide encoding a laccase similar to SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, or SEQ ID NO:104; f) a polynucleotide encoding a benzoyl transferase similar to SEQ ID NO:106 or SEQ ID NO:108; g) a polynucleotide encoding a rhamnosyltransferase similar to SEQ ID NO:110, SEQ ID NO:112, SEQ ID NO:114, or SEQ ID NO:116; h) a polynucleotide encoding an isoflavone-7-O-methyltransferase similar to SEQ ID NO:118, SEQ ID NO:120, SEQ ID NO:122, SEQ ID NO:124, or SEQ ID NO:126; and i) a polynucleotide encoding an AUX/IAA protein similar to SEQ ID NO:128, SEQ ID NO:130, SEQ ID NO:132, or SEQ ID NO:134. Preferably, the promoter is a constitutive promoter. More preferably, the promoter is capable of specifically directing expression in plant roots. Most preferably, the promoter is capable of specifically directing expression in a syncytia site of a plant infected with nematodes.

In another embodiment, the invention provides a method of producing a nematode-resistant transgenic plant, wherein the method comprises the steps of: a) transforming a wild type plant cell with an expression vector comprising a promoter operably linked to a polynucleotide selected from the group consisting of: a) a polynucleotide encoding an AP2/EREBP transcription factor similar to SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, or SEQ ID NO:20; b) a polynucleotide encoding a harpin-induced protein similar to SEQ ID NO:22, SEQ ID NO:24, SEQ ID NO:26, SEQ ID NO:28, SEQ ID NO:30, SEQ ID NO:32, SEQ ID NO:34, SEQ ID NO:36, or SEQ ID NO:38; c) a polynucleotide encoding a TINY-like transcription factor similar to SEQ ID NO:40, SEQ ID NO:42, SEQ ID NO:44, SEQ ID NO:46, or SEQ ID NO:48; d) a polynucleotide encoding an annexin protein similar to SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, or SEQ ID NO:78; e) a polynucleotide encoding a laccase similar to SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, or SEQ ID NO:104; f) a polynucleotide encoding a benzoyl transferase similar to SEQ ID NO:106 or SEQ ID NO:108; g) a polynucleotide encoding a rhamnosyltransferase similar to SEQ ID NO:110, SEQ ID NO:112, SEQ ID NO:114, or SEQ ID NO:116; h) a polynucleotide encoding an isoflavone-7-O-methyltransferase similar to SEQ ID NO:118, SEQ ID NO:120, SEQ ID NO:122, SEQ ID NO:124, or SEQ ID NO:126; and i) a polynucleotide encoding an AUX/IAA protein similar to SEQ ID NO:128, SEQ ID NO:130, SEQ ID NO:132, or SEQ ID NO:134; b) regenerating transgenic plants from the transformed plant cell; and c) selecting transgenic plants for increased nematode resistance as compared to a control plant of the same species.

In another embodiment, the invention provides a method of increasing yield of a crop plant, the method comprising the steps of transforming a plant cell with an expression vector comprising a promoter operably linked to a polynucleotide encoding an AP2/EREBP transcription factor similar to SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, or SEQ ID NO:20; regenerating transgenic plants from the transformed plant cell, and selecting transgenic plants for increased root growth as compared to a control plant of the same species.

### BRIEF DECRIPTION OF THE DRAWINGS

FIG. 1 shows the table of SEQ ID NOs assigned to corresponding polynucleotides and promoters.

FIG. 2 shows an amino acid alignment of exemplary AP2/EREBP transcription factors suitable for use in the present invention. The alignment is performed in Vector NTI software suite (gap opening penalty=10, gap extension penalty=0.05, gap separation penalty=8).

FIG. 3 shows an amino acid alignment of exemplary harpin-induced proteins suitable for use in the present invention. The alignment is performed in Vector NTI software suite (gap opening penalty=10, gap extension penalty=0.05, gap separation penalty=8).

FIG. 4 shows an amino acid alignment of exemplary TINY-like transcription factors suitable for use in the present invention. The alignment is performed in Vector NTI software suite (gap opening penalty=10, gap extension penalty=0.05, gap separation penalty=8).

FIG. 5a-5b shows an amino acid alignment of exemplary annexin proteins suitable for use in the present invention. The alignment is performed in Vector NTI software suite (gap opening penalty=10, gap extension penalty=0.05, gap separation penalty=8).

FIG. 6a-6c shows an amino acid alignment of exemplary laccase proteins suitable for use in the present invention. The alignment is performed in Vector NTI software suite (gap opening penalty=10, gap extension penalty=0.05, gap separation penalty=8).

FIG. 7 shows an amino acid alignment of exemplary benzoyl transferases suitable for use in the present invention. The alignment is performed in Vector NTI software suite (gap opening penalty=10, gap extension penalty=0.05, gap separation penalty=8).

FIG. 8 shows an amino acid alignment of exemplary anthocyanidin-3-glucoside rhamnosyltransferases suitable for use in the present invention The alignment is performed in Vector NTI software suite (gap opening penalty=10, gap extension penalty=0.05, gap separation penalty=8).

FIG. 9 shows an amino acid alignment of exemplary isoflavone-7-O-methyltransferases suitable for use in the present invention. The alignment is performed in Vector NTI software suite (gap opening penalty=10, gap extension penalty=0.05, gap separation penalty=8).

FIG. 10 shows an amino acid alignment of exemplary AUX/IAA proteins suitable for use in the present invention. The alignment is performed in Vector NTI software suite (gap opening penalty=10, gap extension penalty=0.05, gap separation penalty=8).

### DETAILED DESCRIPTION OF THE PREFERRED EMBODIMENTS

The present invention may be understood more readily by reference to the following detailed description and the examples included herein. Throughout this application, various publications are referenced. The disclosures of all of these publications and those references cited within those publications in their entireties are hereby incorporated by reference into this application in order to more fully describe the state of the art to which this invention pertains. The terminology used herein is for the purpose of describing specific embodiments only and is not intended to be limiting. As used herein, “a” or “an” can mean one or more, depending upon the context in which it is used. Thus, for example, reference to “a cell” can mean that at least one cell can be used. As used herein, the word “or” means any one member of a particular list and also includes any combination of members of that list.

As defined herein, a “transgenic plant” is a plant that has been altered using recombinant DNA technology to contain an isolated nucleic acid which would otherwise not be present in the plant. As used herein, the term “plant” includes a whole plant, plant cells, and plant parts. Plant parts include, but are not limited to, stems, roots, ovules, stamens, leaves, embryos, meristematic regions, callus tissue, gametophytes, sporophytes, pollen, microspores, and the like.

As defined herein, the term “nucleic acid” and “polynucleotide” are interchangeable and refer to RNA or DNA that is linear or branched, single or double stranded, or a hybrid thereof. The term also encompasses RNA/DNA hybrids. An “isolated” nucleic acid molecule is one that is substantially separated from other nucleic acid molecules which are present in the natural source of the nucleic acid (i.e., sequences encoding other polypeptides). For example, a cloned nucleic acid is considered isolated. A nucleic acid is also considered isolated if it has been altered by human intervention, or placed in a locus or location that is not its natural site, or if it is introduced into a cell by transformation. Moreover, an isolated nucleic acid molecule, such as a cDNA molecule, can be free from some of the other cellular material with which it is naturally associated, or culture medium when produced by recombinant techniques, or chemical precursors or other chemicals when chemically synthesized. While it may optionally encompass untranslated sequence located at both the 3′ and 5′ ends of the coding region of a gene, it may be preferable to remove the sequences which naturally flank the coding region in its naturally occurring replicon.

The term “gene” is used broadly to refer to any segment of nucleic acid associated with a biological function. Thus, genes include introns and exons as in genomic sequence, or just the coding sequences as in cDNAs and/or the regulatory sequences required for their expression. For example, gene refers to a nucleic acid fragment that expresses mRNA or functional RNA, or encodes a specific protein, and which includes regulatory sequences.

The terms “polypeptide” and “protein” are used interchangeably herein to refer to a polymer of consecutive amino acid residues.

The terms “operably linked” and “in operative association with” are interchangeable and as used herein refer to the association of isolated polynucleotides on a single nucleic acid fragment so that the function of one isolated polynucleotide is affected by the other isolated polynucleotide. For example, a regulatory DNA is said to be “operably linked to” a DNA that expresses an RNA or encodes a polypeptide if the two DNAs are situated such that the regulatory DNA affects the expression of the coding DNA.

The term “promoter” as used herein refers to a DNA sequence which, when ligated to a nucleotide sequence of interest, is capable of controlling the transcription of the nucleotide sequence of interest into mRNA. A promoter is typically, though not necessarily, located 5′ (e.g., upstream) of a nucleotide of interest (e.g., proximal to the transcriptional start site of a structural gene) whose transcription into mRNA it controls, and provides a site for specific binding by RNA polymerase and other transcription factors for initiation of transcription.

The term “transcription regulatory element” as used herein refers to a polynucleotide that is capable of regulating the transcription of an operably linked polynucleotide. It includes, but not limited to, promoters, enhancers, introns, 5′ UTRs, and 3′ UTRs.

As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. In the present specification, “plasmid” and “vector” can be used interchangeably as the plasmid is the most commonly used form of vector. A vector can be a binary vector or a T-DNA that comprises the left border and the right border and may include a gene of interest in between. The term “expression vector” is interchangeable with the term “transgene” as used herein and means a vector capable of directing expression of a particular nucleotide in an appropriate host cell. The expression of the nucleotide can be over-expression. An expression vector comprises a regulatory nucleic acid element operably linked to a nucleic acid of interest, which is—optionally—operably linked to a termination signal and/or other regulatory element.

The term “homologs” as used herein refers to a gene related to a second gene by descent from a common ancestral DNA sequence. The term “homologs” may apply to the relationship between genes separated by the event of speciation (e.g., orthologs) or to the relationship between genes separated by the event of genetic duplication (e.g., paralogs).

As used herein, the term “orthologs” refers to genes from different species, but that have evolved from a common ancestral gene by speciation. Orthologs retain the same function in the course of evolution. Orthologs encode proteins having the same or similar functions. As used herein, the term “paralogs” refers to genes that are related by duplication within a genome. Paralogs usually have different functions or new functions, but these functions may be related.

The term “conserved region” or “conserved domain” as used herein refers to a region in heterologous polynucleotide or polypeptide sequences where there is a relatively high degree of sequence identity between the distinct sequences. The “conserved region” can be identified, for example, from the multiple sequence alignment using the Clustal W algorithm.

The term “cell” or “plant cell” as used herein refers to single cell, and also includes a population of cells. The population may be a pure population comprising one cell type. Likewise, the population may comprise more than one cell type. A plant cell within the meaning of the invention may be isolated (e.g., in suspension culture) or comprised in a plant tissue, plant organ or plant at any developmental stage.

The term “true breeding” as used herein refers to a variety of plant for a particular trait if it is genetically homozygous for that trait to the extent that, when the true-breeding variety is self-pollinated, a significant amount of independent segregation of the trait among the progeny is not observed.

The term “null segregant” as used herein refers to a progeny (or lines derived from the progeny) of a transgenic plant that does not contain the transgene due to Mendelian segregation.

The term “wild type” as used herein refers to a plant cell, seed, plant component, plant tissue, plant organ, or whole plant that has not been genetically modified or treated in an experimental sense.

The term “control plant” as used herein refers to a plant cell, an explant, seed, plant component, plant tissue, plant organ, or whole plant used to compare against transgenic or genetically modified plant for the purpose of identifying an enhanced phenotype or a desirable trait in the transgenic or genetically modified plant. A “control plant” may in some cases be a transgenic plant line that comprises an empty vector or marker gene, but does not contain the recombinant polynucleotide of interest that is present in the transgenic or genetically modified plant being evaluated. A control plant may be a plant of the same line or variety as the transgenic or genetically modified plant being tested, or it may be another line or variety, such as a plant known to have a specific phenotype, characteristic, or known genotype. A suitable control plant would include a genetically unaltered or non-transgenic plant of the parental line used to generate a transgenic plant herein.

The term “syncytia site” as used herein refers to the feeding site formed in plant roots after nematode infestation. The site is used as a source of nutrients for the nematodes. A syncytium is the feeding site for cyst nematodes and giant cells are the feeding sites of root knot nematodes.

Crop plants and corresponding parasitic nematodes are listed in Index of Plant Diseases in the United States (U.S. Dept. of Agriculture Handbook No. 165, 1960); Distribution of Plant-Parasitic Nematode Species in North America (Society of Nematologists, 1985); and Fungi on Plants and Plant Products in the United States (American Phytopathological Society, 1989). For example, plant parasitic nematodes that are targeted by the present invention include, without limitation, cyst nematodes and root-knot nematodes. Specific plant parasitic nematodes which are targeted by the present invention include, without limitation, Heterodera glycines, Heterodera schachtii, Heterodera avenae, Heterodera oryzae, Heterodera cajani, Heterodera trifolii, Globodera pallida, G. rostochiensis, or Globodera tabacum, Meloidogyne incognita, M. arenaria, M. hapla, M. javanica, M. naasi, M. exigua, Ditylenchus dipsaci, Ditylenchus angustus, Radopholus similis, Radopholus citrophilus, Helicotylenchus multicinctus, Pratylenchus coffeae, Pratylenchus brachyurus, Pratylenchus vulnus, Paratylenchus curvitatus, Paratylenchus zeae, Rotylenchulus reniformis, Paratrichodorus anemones, Paratrichodorus minor, Paratrichodorus christiei, Anguina tritici, Bidera avenae, Subanguina radicicola, Hoplolaimus seinhorsti, Hoplolaimus Columbus, Hoplolaimus galeatus, Tylenchulus semipenetrans, Hemicycliophora arenaria, Rhadinaphelenchus cocophilus, Belonolaimus longicaudatus, Trichodorus primitivus, Nacobbus aberrans, Aphelenchoides besseyi, Hemicriconemoides kanayaensis, Tylenchorhynchus claytoni, Xiphinema americanum, Cacopaurus pestis, Heterodera zeae, Heterodera filipjevi and the like.

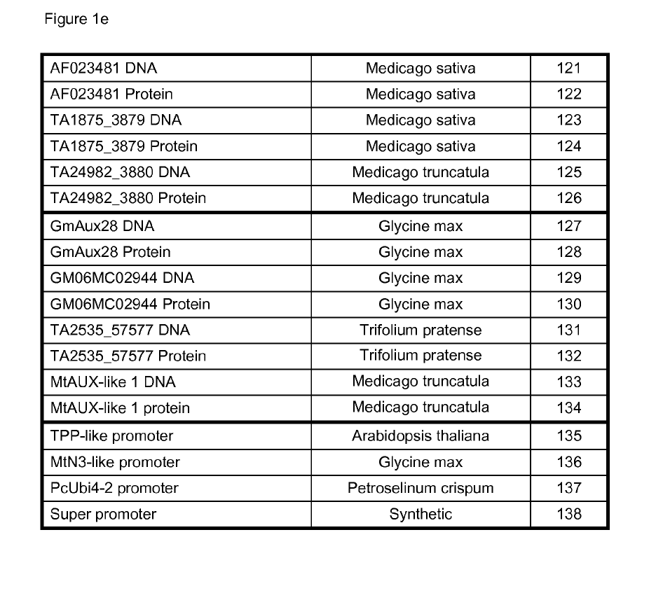

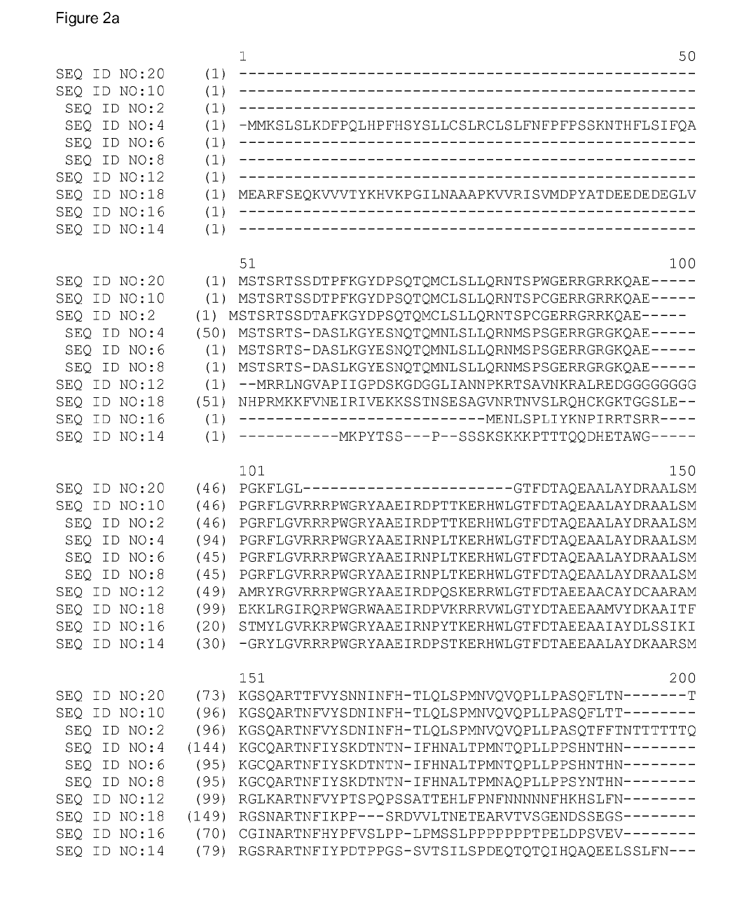

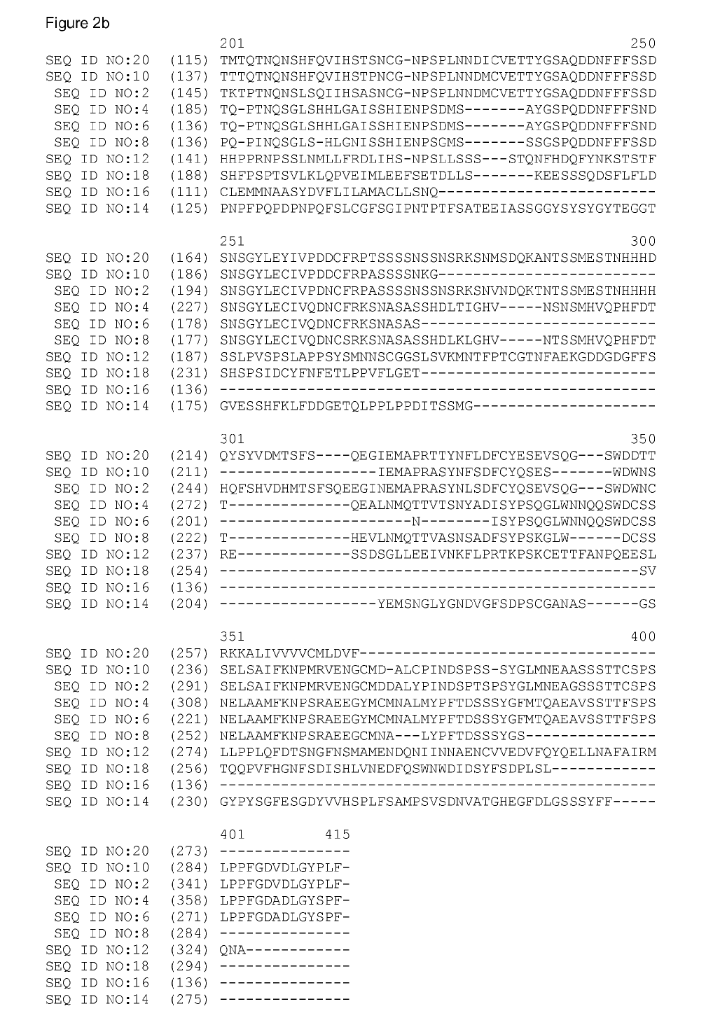

In one embodiment, the invention provides a transgenic plant transformed with an expression vector comprising an isolated polynucleotide that encodes an AP2/EREBP domain-containing transcription factor that is similar to the transcription factors set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, or SEQ ID NO:20. As described in Examples 1 and 2 below, transgenic soybean root lines expressing the AP2/EREBP polynucleotides having SEQ ID NOs:1, 3, and 7, respectively, demonstrated increased resistance to nematode infection as compared to control lines. An amino acid alignment of several exemplary AP2/EREBP domain-containing transcription factors which are suitable for use in the present embodiment is shown in FIG. 2. Any polynucleotide encoding a protein comprising an AP2/EREBP domain similar to the AP2/EREBP domains of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, or SEQ ID NO:20 may be used as described herein to produce a nematode-resistant transgenic plant. For example, polynucleotides encoding any of the AP2/EREBP domain-containing proteins set forth in FIG. 2 may be transformed into a nematode-susceptible plant to produce a nematode-resistant transgenic plant.

As set forth in Example 3 below, transgenic soybean root lines expressing the AP2/EREBP proteins encoded by SEQ ID NOs:1 and 7 also demonstrated increased root weight as compared to control lines. Root architecture has been associated with yield in several crops. For example, retrospective analyses of the physiological basis of genetic yield improvement in maize have shown that newer maize hybrids tolerate higher planting density better than commercial hybrids from earlier decades and that this change explains much of the genetic gain for yield that was accomplished by plant breeding over the past several decades. The ability of plants to tolerate the inter-plant competition associated with higher planting density is a form of stress tolerance. This stress tolerance and the consequent yield improvement have been shown to be the result of more efficient capture and use of resources from the environment to support plant growth and development. Differences in canopy architecture and longevity of leaves enable more light (energy) to be captured during the life cycle of the plant resulting in greater photosynthesis and this in turn enables more carbohydrates to be produced and stored as biomass or in seed. In addition, a more efficient root system enables greater uptake of nutrients and water under the more competitive conditions associated with higher planting density. Recent computer simulation studies, which were validated by field experiments, indicate that a change in root system architecture which increases water capture has a greater and more direct effect on biomass accumulation and maize yield than changes in canopy architecture.

The relationship between plant size and the uptake of water by roots is predicted based on the biophysics of plant growth. Plants grow by the expansion of cells. This is driven osmotically by differences in water potential between the interior and exterior of the cell and is resisted by the cell wall's elasticity or ability to expand. The water potential gradient is created by a gradient of osmotically-active solutes including potassium and other nutrients obtained from the soil. Therefore, cell expansion can be limited by either mechanical or hydraulic constraints or both. The hydraulic constraints due to a restriction in the amount of water or osmotically-active nutrients may be caused either by a lack of their availability in the soil (e.g. drought) or by a lack of root penetration into the regions of the soil that contain water and nutrients.

Roots are also important to maintain the plant in an upright position at maturity to enable harvesting. Lodging can occur due to stalk breakage or due to upheaval of the plant from the soil. In maize, improvement in crown root numbers or in the extent of root branching would improve stand establishment and standability especially if grown in high planting densities. Therefore in maize, improved root properties, including architecture, branching, and soil penetration, are anticipated to provided increased acquisition of water and nutrients to support cell expansion, increased nutrient uptake to support metabolism including protein synthesis and reduced lodging resulting in increased harvestable yield. To facilitate nutrient and water uptake, plants have also evolved the formation of microscopic projections from epidermal cells of the root surfaces known as root hairs. Root hairs enlarge the surface of the root by as much as 77% in crop plants to support uptake of water and nutrients and affect the interaction with abiotic and biotic rhizosphere. Root hairs have been shown to play a substantial role in affecting yields especially in maize. Variations in root hair number, size and shape can lead to striking effects on the plants ability to optimally uptake water and nutrients. With dramatically reduced root hair development, yields in maize can show losses of up to approximately 40%, indicating that increased role root hair growth contributes to overall grain yield.

Accordingly, polynucleotides encoding AP2/EREBP proteins that are similar to the AP2/EREBP domain-containing transcription factors of FIG. 2 may also be used to improve yield of crop plants. As used herein, the term “improved yield” means any improvement in the yield of any measured plant product, such as grain, fruit or fiber. In accordance with the invention, changes in different phenotypic traits may improve yield. For example, and without limitation, parameters such as floral organ development, root initiation, root biomass, seed number, seed weight, harvest index, tolerance to abiotic environmental stress, reduction of nutrient, e.g., nitrogen or phosphorus, input requirement, leaf formation, phototropism, apical dominance, and fruit development, are suitable measurements of improved yield. Any increase in yield is an improved yield in accordance with the invention. For example, the improvement in yield can comprise a 0.1%, 0.5%, 1%, 3%, 5%, 10%, 15%, 20%, 30%, 40%, 50%, 60%, 70%, 80%, 90% or greater increase in any measured parameter. For example, an increase in the bu/acre yield of soybeans or corn derived from a crop comprising plants which are transgenic for the AP2/EREBP domain-containing transcription factors described herein, as compared with the bu/acre yield from untreated soybeans or corn cultivated under the same conditions, is an improved yield in accordance with the invention.

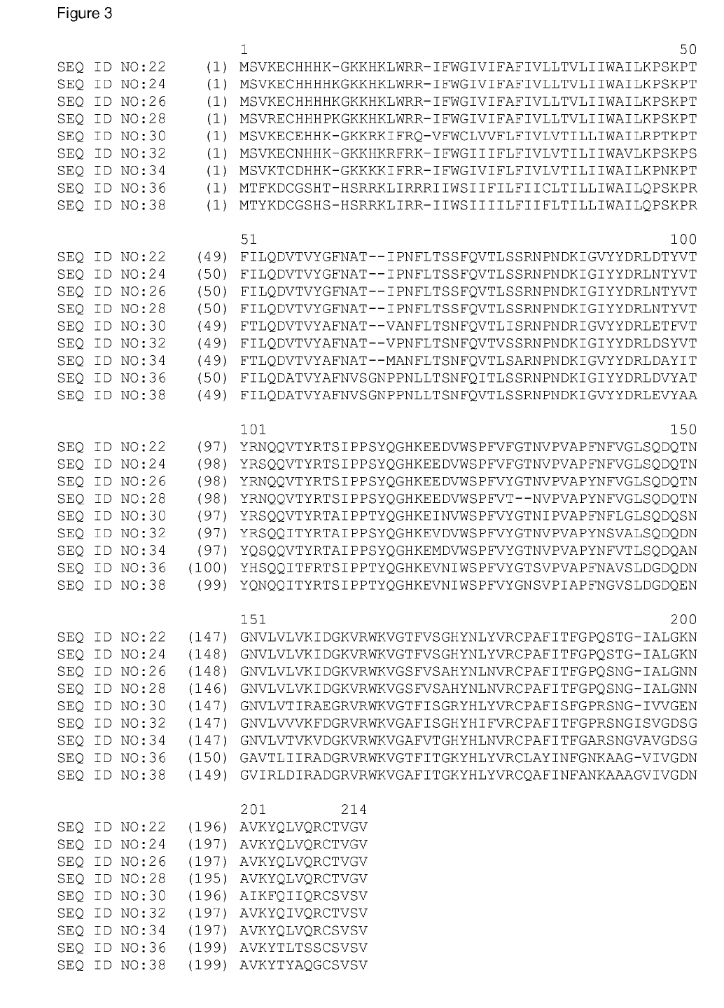

In another embodiment, the invention provides a transgenic plant transformed with an expression vector comprising an isolated polynucleotide that encodes a harpin-induced protein similar to the polypeptides set forth in SEQ ID NO:22, SEQ ID NO:24, SEQ ID NO:26, SEQ ID NO:28, SEQ ID NO:30, SEQ ID NO:32, SEQ ID NO:34, SEQ ID NO:36 or SEQ ID NO:38. As described in Examples 1 and 2 below, transgenic soybean root lines expressing the harpin-induced polynucleotide having SEQ ID NO:21 demonstrated increased resistance to nematode infection as compared to control lines. An amino acid alignment of several exemplary harpin-induced polypeptides which are suitable for use in this embodiment is set forth in FIG. 3. Any polynucleotide encoding a protein similar to the harpin-induced proteins set forth in SEQ ID NO:22, SEQ ID NO:24, SEQ ID NO:26, SEQ ID NO:28, SEQ ID NO:30, SEQ ID NO:32, SEQ ID NO:34, SEQ ID NO:36 and SEQ ID NO:38, may be used as described herein to produce a nematode-resistant transgenic plant. For example, polynucleotides encoding any of the harpin-induced proteins set forth in FIG. 3 may be transformed into a nematode-susceptible plant to produce a nematode-resistant transgenic plant.

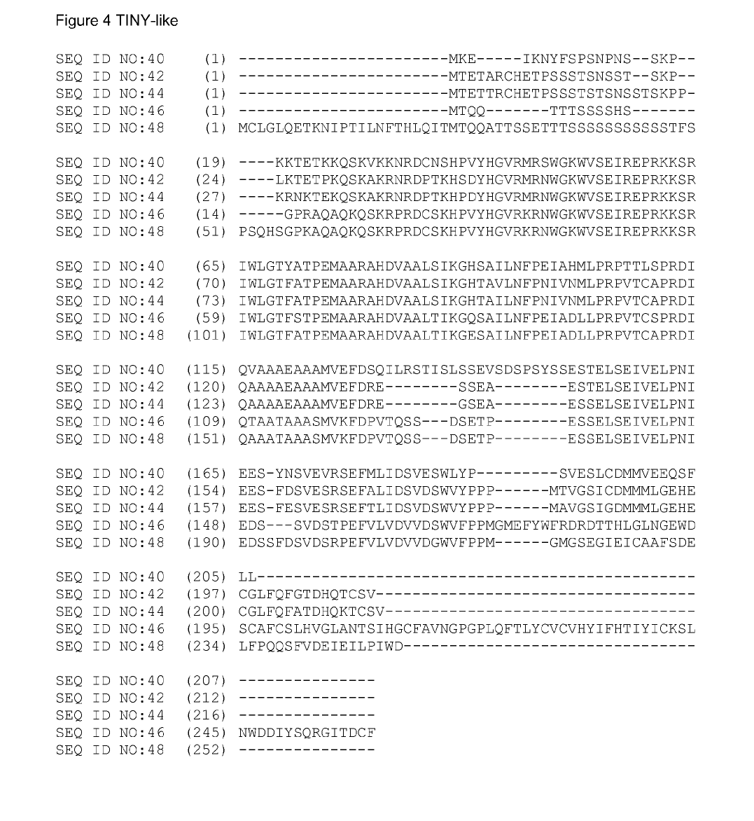

In another embodiment, the invention provides a transgenic plant transformed with an expression vector comprising an isolated polynucleotide that encodes a TINY-like transcription factor similar to the polypeptides set forth in SEQ ID NO:40, SEQ ID NO:42, SEQ ID NO:44, SEQ ID NO:46 and SEQ ID NO:48. As described in Examples 1 and 2 below, transgenic soybean root lines expressing the M. trunculata TINY-like transcription factor polynucleotide having SEQ ID NO:39 demonstrated increased resistance to nematode infection as compared to control lines. An amino acid alignment of exemplary TINY-like transcription factors suitable for use in this embodiment is set forth in FIG. 4. Any polynucleotide encoding a protein similar to the TINY-like transcription factor proteins of SEQ ID NO:40, SEQ ID NO:42, SEQ ID NO:44, SEQ ID NO:46 or SEQ ID NO:48 may be used as described herein to produce a nematode-resistant transgenic plant. For example, polynucleotides encoding any of the TINY-like transcription factor proteins set forth in FIG. 4 may be transformed into a wild-type plant to produce a nematode-resistant transgenic plant.

In another embodiment, the invention provides a transgenic plant transformed with an expression vector comprising an isolated polynucleotide that encodes an annexin similar to the annexins set forth in SEQ ID NO:50: SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76 and SEQ ID NO:78. As described in Examples 1 and 2 below, transgenic soybean root lines expressing the G. max annexin polynucleotide having SEQ ID NO:49 demonstrated increased resistance to nematode infection as compared to control lines. An amino acid alignment of several exemplary annexins suitable for use in this embodiment is set forth in FIG. 5. Any polynucleotide encoding an annexin similar to the protein of SEQ ID NO:50: SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76 or SEQ ID NO:78 may be used as described herein to produce a nematode-resistant transgenic plant. For example, polynucleotides encoding any of the annexin proteins set forth in FIG. 5 may be transformed into a nematode-susceptible plant to produce a nematode-resistant transgenic plant.

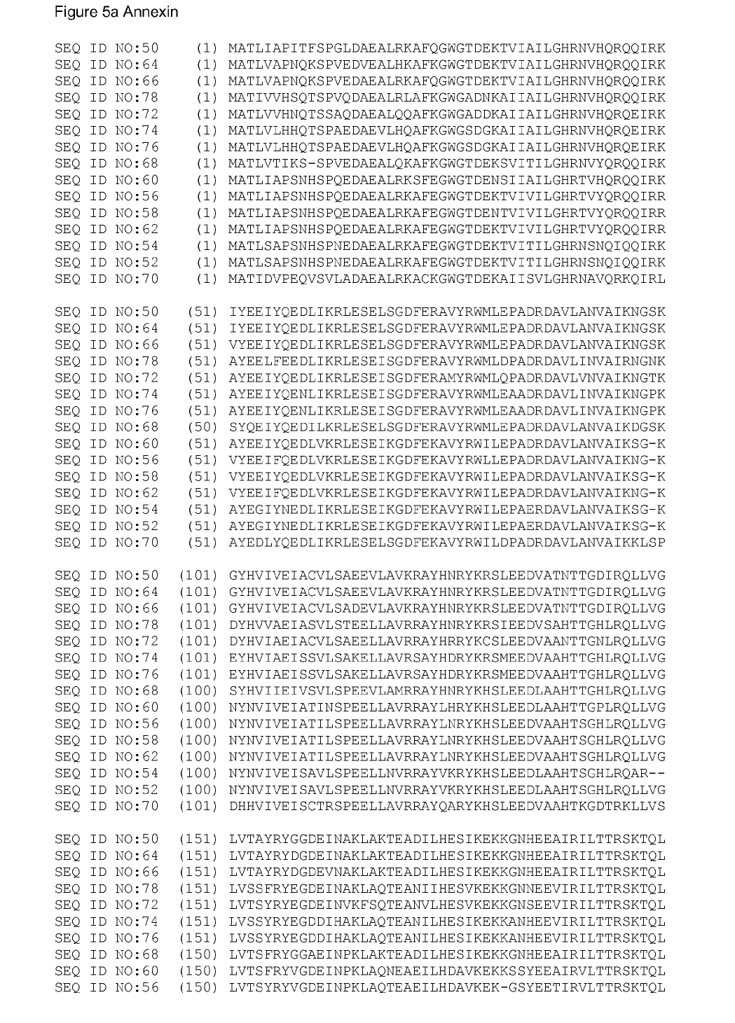

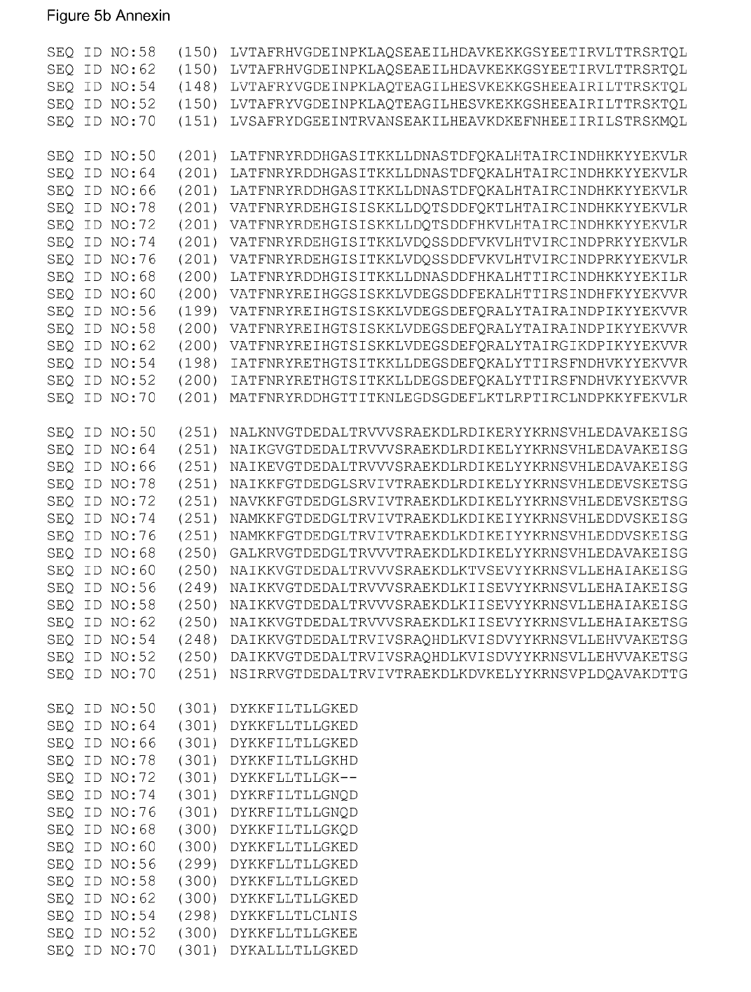

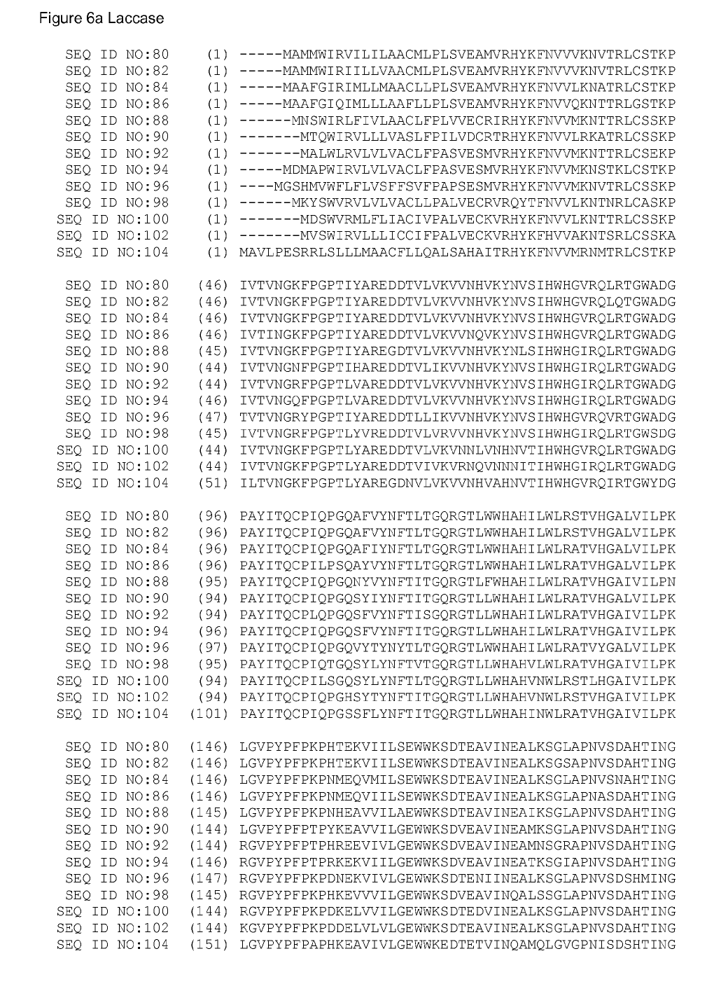

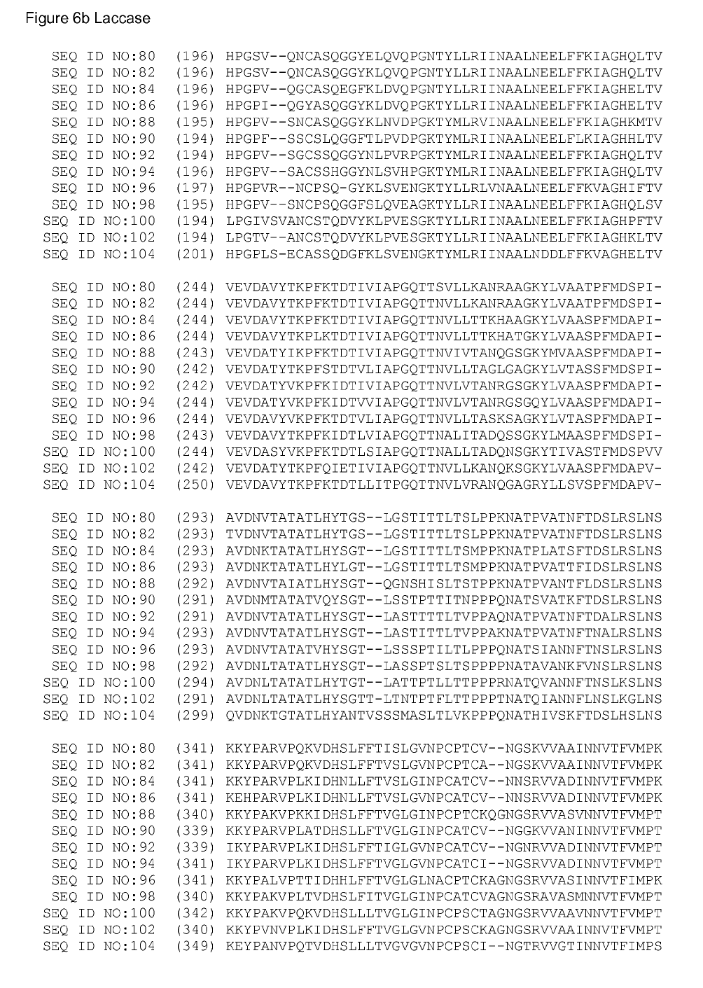

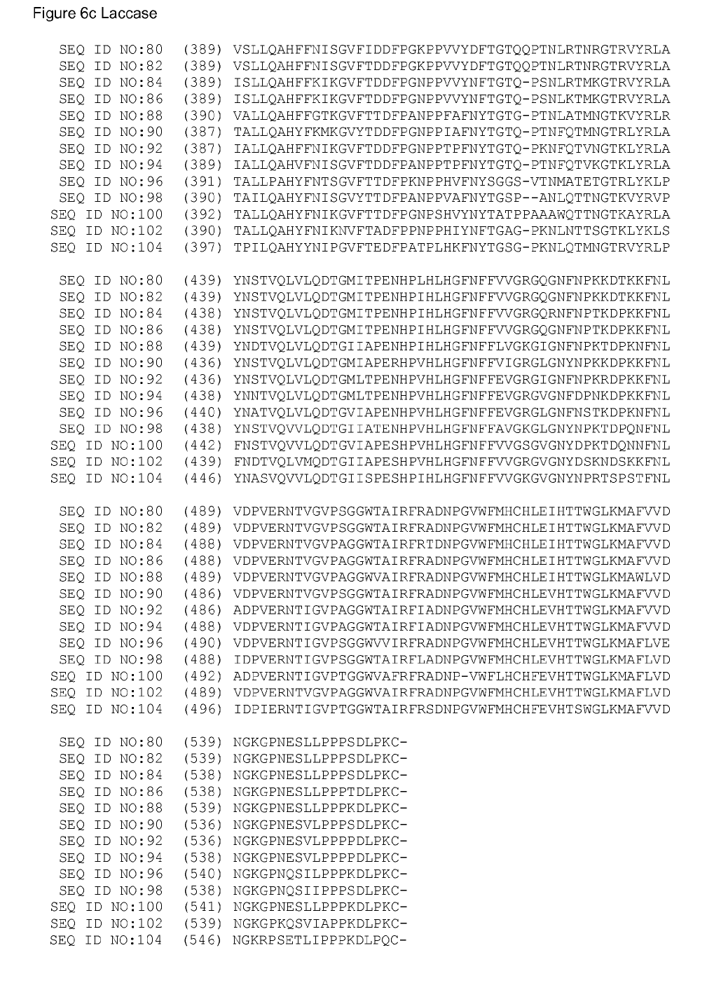

In another embodiment, the invention provides a transgenic plant transformed with an expression vector comprising an isolated polynucleotide that encodes a laccase similar to the laccases set forth in SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98 SEQ ID NO:100, SEQ ID NO:102 and SEQ ID NO:104. As described in Examples 1 and 2 below, transgenic soybean root lines expressing the G. max laccase polynucleotide having SEQ ID NO:79 demonstrated increased resistance to nematode infection as compared to control lines. An alignment of several exemplary laccases suitable for use in this embodiment is set forth in FIG. 6. Any polynucleotide encoding a laccase similar to the protein of SEQ ID NO:80 may be used as described herein to produce a nematode-resistant transgenic plant. For example, polynucleotides encoding any of the laccase proteins set forth in FIG. 6 may be transformed into a nematode-susceptible plant to produce a nematode-resistant transgenic plant.

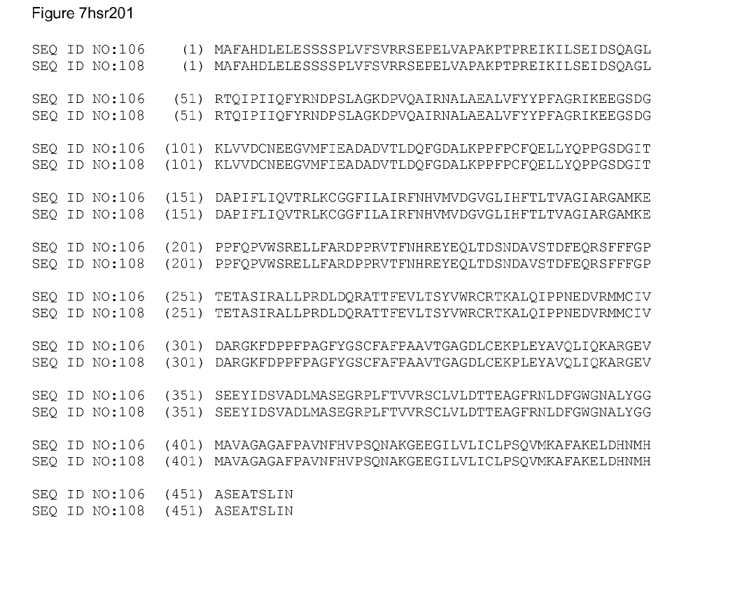

In another embodiment, the invention provides a transgenic plant transformed with an expression vector comprising an isolated polynucleotide that encodes a benzoyl-CoA:benzyl alcohol/phenylethanol benzoyltransferase similar to the polypeptides set forth in SEQ ID NO:106 and SEQ ID NO:108. As described in Examples 1 and 2 below, transgenic soybean root lines expressing the G. max benzoyl-CoA:benzyl alcohol/phenylethanol benzoyltransferase polynucleotide having SEQ ID NO:105 demonstrated increased resistance to nematode infection as compared to control lines. An alignment of exemplary benzoyltransferases suitable for use in this embodiment is set forth in FIG. 7. Any polynucleotide encoding a benzoyltransferase similar to the proteins of SEQ ID NO:106 or SEQ ID NO:108 may be used as described herein to produce a nematode-resistant transgenic plant. For example, polynucleotides encoding any of the benzoyltransferase proteins set forth in FIG. 7 may be transformed into a nematode-susceptible plant to produce a nematode-resistant transgenic plant.

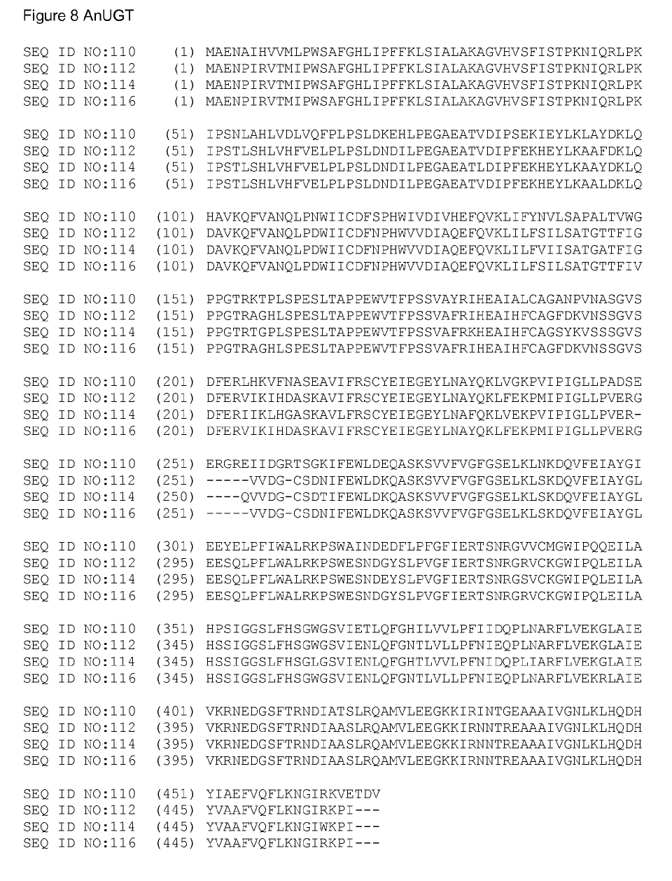

In another embodiment, the invention provides a transgenic plant transformed with an expression vector comprising an isolated polynucleotide that encodes an anthocyanidin-3-glucoside rhamnosyltransferase similar to the rhamnosyltransferases set forth in SEQ ID NO:110, SEQ ID NO:112, SEQ ID NO:114 and SEQ ID NO:116. As described in Examples 1 and 2 below, transgenic soybean root lines expressing the G. max anthocyanidin-3-glucoside rhamnosyltransferase polynucleotide having SEQ ID NO:109 demonstrated increased resistance to nematode infection as compared to control lines. An alignment of several exemplary rhamnosyltransferases suitable for use in this embodiment is set forth in FIG. 8. Any polynucleotide encoding a rhamnosyltransferase similar to those of SEQ ID NO:110, SEQ ID NO:112, SEQ ID NO:114 or SEQ ID NO:116 may be used as described herein to produce a nematode-resistant transgenic plant. For example, polynucleotides encoding any of the laccase proteins set forth in FIG. 8 may be transformed into a nematode-susceptible plant to produce a nematode-resistant transgenic plant.

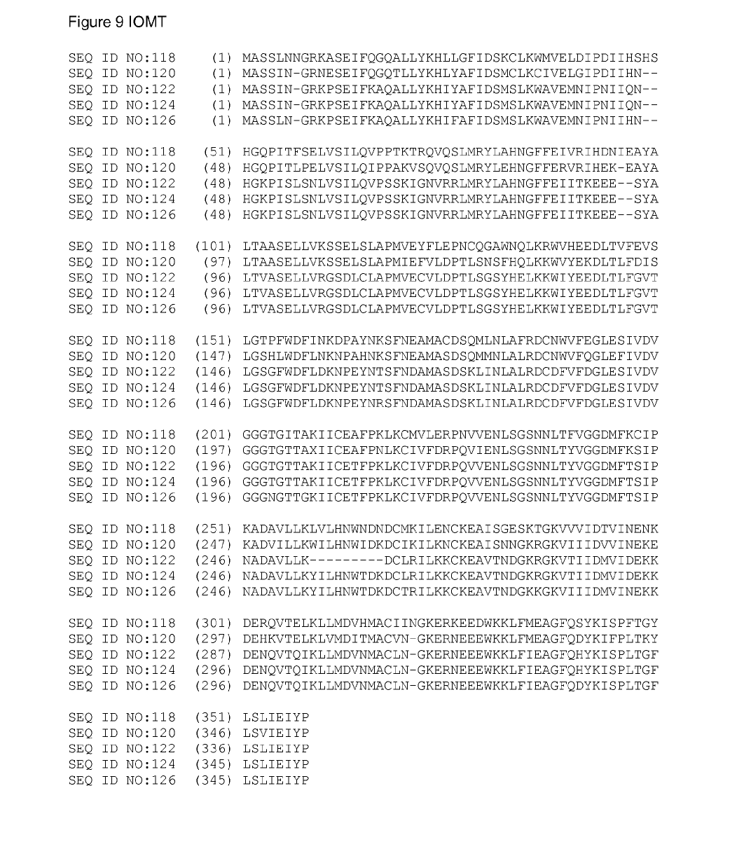

In another embodiment, the invention provides a transgenic plant transformed with an expression vector comprising an isolated polynucleotide that encodes an isoflavone-7-O-methyltransferase similar to the methyltransferases set forth in SEQ ID NO:118, SEQ ID NO:120, SEQ ID NO:122, SEQ ID NO:124 and SEQ ID NO:126. As described in Examples 1 and 2 below, transgenic soybean root lines expressing the G. max isoflavone-7-O-methyltransferase polynucleotide having SEQ ID NO:117 demonstrated increased resistance to nematode infection as compared to control lines. An alignment of exemplary isoflavone-7-O-methyltransferases suitable for use in this embodiment is set forth in FIG. 9. Any polynucleotide encoding a methyltransferase similar to the proteins of SEQ ID NO:118, SEQ ID NO:120, SEQ ID NO:122, SEQ ID NO:124 and SEQ ID NO:126 may be used as described herein to produce a nematode-resistant transgenic plant. For example, polynucleotides encoding any of the isoflavone-7-O-methyltransferase proteins set forth in FIG. 9 may be transformed into a nematode-susceptible plant to produce a nematode-resistant transgenic plant.

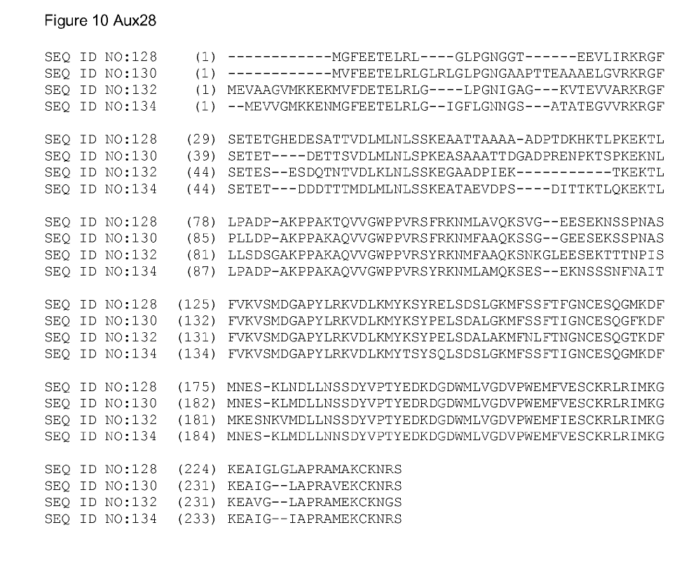

In another embodiment, the invention provides a transgenic plant transformed with an expression vector comprising an isolated polynucleotide that encodes an AUX/IAA polypeptide similar to the AUX/IAA proteins set forth in SEQ ID NO:128, SEQ ID NO:130, SEQ ID NO:132 and SEQ ID NO:134. As described in Examples 1 and 2 below, transgenic soybean root lines expressing the G. max AUX/IAA polynucleotide having SEQ ID NO:127 demonstrated increased resistance to nematode infection as compared to control lines. An alignment of exemplary AUX/IAA proteins suitable for use in this embodiment is set forth in FIG. 10. Any polynucleotide encoding an AUX/IAA protein similar to the AUX/IAA proteins of SEQ ID NO:128, SEQ ID NO:130, SEQ ID NO:132 and SEQ ID NO:134 may be used as described herein to produce a nematode-resistant transgenic plant. For example, polynucleotides encoding any of the AUX/IAA proteins set forth in FIG. 10 may be transformed into a nematode-susceptible plant to produce a nematode-resistant transgenic plant.

The transgenic plant of the invention may be characterized as a monocotyledonous plant or a dicotyledonous plant. For example and without limitation, transgenic plants of the invention may be maize, wheat, rice, barley, oat, rye, sorghum, banana, ryegrass, pea, alfalfa, soybean, carrot, celery, tomato, potato, cotton, tobacco, pepper, oilseed rape, sugar beet, cabbage, cauliflower, broccoli, lettuce. A. thaliana, rose, or any plant species which is amenable to transformation. The transgenic plant of the invention may be male sterile or male fertile, and may further include transgenes other than those that comprise the isolated polynucleotides described herein.

The transgenic plants of the invention may be crossed with similar transgenic plants or with transgenic plants lacking the polynucleotides described above or with non-transgenic plants, using known methods of plant breeding, to prepare seeds. The present invention also provides seed and parts from the transgenic plants described above, and progeny plants from such plants, including hybrids and inbreds. The invention also provides a method of plant breeding, e.g., to prepare a crossed fertile transgenic plant. The method comprises crossing a fertile transgenic plant comprising a particular expression vector of the invention with itself or with a second plant, e.g., one lacking the particular expression vector, to prepare the seed of a crossed fertile transgenic plant comprising the particular expression vector. The seed is then planted to obtain a crossed fertile transgenic plant. The crossed fertile transgenic plant may have the particular expression vector inherited through a female parent or through a male parent. The second plant may be an inbred plant. The crossed fertile transgenic plant may be a hybrid. Also included within the present invention are seeds of any of these crossed fertile transgenic plants. The seeds of this invention can be harvested from fertile transgenic plants and be used to grow progeny generations of transformed plants of this invention including hybrid plant lines comprising the nematode resistance-conferring polynucleotides described above.

In accordance with the invention, nematode-resistant transgenic plants may be produced by stacking any one of the nematode resistance polynucleotides described herein with at least one other polynucleotide disclosed herein. The transgenic plant of the present invention may comprise, and/or be crossed to another transgenic plant that comprises one or more transgenes, thus creating a “stack” of transgenes (also referred to as a “gene stack”) in the plant and/or its progeny. These stacked combinations can be created by any method including but not limited to cross breeding plants by conventional methods or by genetic transformation. If the traits are stacked by genetic transformation, trait-conferring polynucleotides can be combined sequentially or simultaneously in any order. For example if two polynucleotides are to be introduced, the two sequences can be contained in separate transformation cassettes or on the same transformation cassette. The expression of the sequences can be driven by the same or different promoters.

For example polynucleotides encoding any two or more of the AP2/EREBP transcription factors of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, or SEQ ID NO:20 may be stacked to provide enhanced nematode resistance or enhanced yield. As another example, polynucleotides encoding any two or more of the harpin-induced proteins of SEQ ID NO:22, SEQ ID NO:24, SEQ ID NO:26, SEQ ID NO:28, SEQ ID NO:30, SEQ ID NO:32, SEQ ID NO:34, SEQ ID NO:36 or SEQ ID NO:38 may be stacked to provide enhanced nematode resistance. Alternatively, polynucleotides encoding any two or more of the TINY-like transcription factors of SEQ ID NO:40, SEQ ID NO:42, SEQ ID NO:44, SEQ ID NO:46 and SEQ ID NO:48 may be stacked to provide enhanced nematode resistance. In another stacking embodiment, polynucleotides encoding any two or more of the annexins set forth in SEQ ID NO:50: SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76 and SEQ ID NO:78 may be stacked to provide enhanced nematode resistance. Furthermore, polynucleotides encoding any two or more of the laccases of SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98 SEQ ID NO:100, SEQ ID NO:102 and SEQ ID NO:104 may be stacked to provide enhanced nematode resistance. In another embodiment, polynucleotides encoding any two or more of the benzoyl-CoA:benzyl alcohol/phenylethanol benzoyltransferases of SEQ ID NO:106 and SEQ ID NO:108 may be stacked to provide enhanced nematode resistance. In another embodiment, polynucleotides encoding any two or more of the anthocyanidin-3-glucoside rhamnosyltransferases of SEQ ID NO:110, SEQ ID NO:112, SEQ ID NO:114 and SEQ ID NO:116 may be stacked to provide enhanced nematode resistance. Polynucleotides encoding any two or more of the isoflavone-7-O-methyltransferases of SEQ ID NO:118, SEQ ID NO:120, SEQ ID NO:122, SEQ ID NO:124 and SEQ ID NO:126 may be stacked to provide enhanced nematode resistance. In another embodiment, polynucleotides encoding any two or more of the AUX/IAA proteins of SEQ ID NO:128, SEQ ID NO:130, SEQ ID NO:132 and SEQ ID NO:134 may be stacked to provide enhanced nematode resistance.

Alternatively, a polynucleotide encoding an AP2/EREBP transcription factor disclosed herein may be stacked with a polynucleotide encoding a harpin-induced protein disclosed herein, a polynucleotide encoding a TINY-like transcription factor disclosed herein, a polynucleotide encoding an annexin disclosed herein, a polynucleotide encoding a laccase disclosed herein, a polynucleotide encoding a benzoyl-CoA:benzyl alcohol/phenylethanol benzoyltransferase disclosed herein, a polynucleotide encoding a anthocyanidin-3-glucoside rhamnosyltransferase disclosed herein, a polynucleotide encoding a isoflavone-7-O-methyltransferase disclosed herein, or a polynucleotide encoding a AUX/IAA protein disclosed herein. Any combination of the polynucleotides disclosed herein may be combined to produce a nematode-resistant plant. In addition, any of the polynucleotides disclosed herein may be combined with any polynucleotide known to enhance resistance to plant parasitic nematodes.

Another embodiment of the invention relates to an expression vector comprising a promoter operably linked to one or more polynucleotides of the invention, wherein expression of the polynucleotide confers increased nematode resistance to a transgenic plant. In one embodiment, the transcription regulatory element is a promoter capable of regulating constitutive expression of an operably linked polynucleotide. A “constitutive promoter” refers to a promoter that is able to express the open reading frame or the regulatory element that it controls in all or nearly all of the plant tissues during all or nearly all developmental stages of the plant. Constitutive promoters include, but are not limited to, the 35S CaMV promoter from plant viruses (Franck et al., Cell 21:285-294, 1980), the Nos promoter (An G. at al., The Plant Cell 3:225-233, 1990), the ubiquitin promoter (Christensen et al., Plant Mol. Biol. 12:619-632, 1992 and 18:581-8,1991), the MAS promoter (Velten et al., EMBO J. 3:2723-30, 1984), the maize H3 histone promoter (Lepetit et al., Mol Gen. Genet 231:276-85, 1992), the ALS promoter (WO96/30530), the 19S CaMV promoter (U.S. Pat. No. 5,352,605), the super-promoter (U.S. Pat. No. 5,955,646), the figwort mosaic virus promoter (U.S. Pat. No. 6,051,753), the rice actin promoter (U.S. Pat. No. 5,641,876), and the Rubisco small subunit promoter (U.S. Pat. No. 4,962,028).

In another embodiment, the transcription regulatory element is a regulated promoter. A “regulated promoter” refers to a promoter that directs gene expression not constitutively, but in a temporally and/or spatially manner, and includes both tissue-specific and inducible promoters. Different promoters may direct the expression of a polynucleotide or regulatory element in different tissues or cell types, or at different stages of development, or in response to different environmental conditions.

A “tissue-specific promoter” or “tissue-preferred promoter” refers to a regulated promoter that is not expressed in all plant cells but only in one or more cell types in specific organs (such as leaves or seeds), specific tissues (such as embryo or cotyledon), or specific cell types (such as leaf parenchyma or seed storage cells). These also include promoters that are temporally regulated, such as in early or late embryogenesis, during fruit ripening in developing seeds or fruit, in fully differentiated leaf, or at the onset of sequence. Suitable promoters include the napin-gene promoter from rapeseed (U.S. Pat. No. 5,608,152), the USP-promoter from Vicia faba (Baeumlein et al., Mol Gen Genet. 225(3):459-67, 1991), the oleosin-promoter from Arabidopsis (WO 98/45461), the phaseolin-promoter from Phaseolus vulgaris (U.S. Pat. No. 5,504,200), the Bce4-promoter from Brassica (WO 91/13980) or the legumin B4 promoter (LeB4; Baeumlein et al., Plant Journal, 2(2):233-9, 1992) as well as promoters conferring seed specific expression in monocot plants like maize, barley, wheat, rye, rice, etc. Suitable promoters to note are the Ipt2 or Ipt1-gene promoter from barley (WO 95/15389 and WO 95/23230) or those described in WO 99/16890 (promoters from the barley hordein-gene, rice glutelin gene, rice oryzin gene, rice prolamin gene, wheat gliadin gene, wheat glutelin gene, maize zein gene, oat glutelin gene, Sorghum kasirin-gene and rye secalin gene). Promoters suitable for preferential expression in plant root tissues include, for example, the promoter derived from corn nicotianamine synthase gene (US 20030131377) and rice RCC3 promoter (U.S. Ser. No. 11/075,113). Suitable promoter for preferential expression in plant green tissues include the promoters from genes such as maize aldolase gene FDA (US 20040216189), aldolase and pyruvate orthophosphate dikinase (PPDK) (Taniguchi et. al., Plant Cell Physiol. 41(1):42-48, 2000).

Inducible promoters” refer to those regulated promoters that can be turned on in one or more cell types by an external stimulus, for example, a chemical, light, hormone, stress, or a nematode such as nematodes. Chemically inducible promoters are especially suitable if gene expression is wanted to occur in a time specific manner. Examples of such promoters are a salicylic acid inducible promoter (WO 95/19443), a tetracycline inducible promoter (Gatz et al., Plant J. 2:397-404, 1992), the light-inducible promoter from the small subunit of Ribulose-1,5-bis-phosphate carboxylase (ssRUBISCO), and an ethanol inducible promoter (WO 93/21334). Also, suitable promoters responding to biotic or abiotic stress conditions are those such as the nematode inducible PRP1-gene promoter (Ward et al., Plant. Mol. Biol. 22:361-366, 1993), the heat inducible hsp80-promoter from tomato (U.S. Pat. No. 5,187,267), cold inducible alpha-amylase promoter from potato (WO 96/12814), the drought-inducible promoter of maize (Busk et. al., Plant J. 11:1285-1295, 1997), the cold, drought, and high salt inducible promoter from potato (Kirch, Plant Mol. Biol. 33:897-909, 1997) or the RD29A promoter from Arabidopsis (Yamaguchi-Shinozalei et. al., Mol. Gen. Genet. 236:331-340, 1993), many cold inducible promoters such as cor15a promoter from Arabidopsis (Genbank Accession No U01377), blt101 and blt4.8 from barley (Genbank Accession Nos AJ310994 and U63993), wcs120 from wheat (Genbank Accession No AF031235), mlip15 from corn (Genbank Accession No D26563), bn115 from Brassica (Genbank Accession No U01377), and the wound-inducible pinll-promoter (European Patent No. 375091).

Of particular utility in the present invention are syncytia site preferred, or nematode feeding site induced, promoters, including, but not limited to promoters from the Mtn3-like promoter disclosed in PCT/EP2008/051328, the Mtn21-like promoter disclosed in PCT/EP2007/051378, the peroxidase-like promoter disclosed in PCT/EP2007/064356, the trehalose-6-phosphate phosphatase-like promoter disclosed in PCT/EP2007/063761 and the At5g12170-like promoter disclosed in PCT/EP2008/051329. All of the forgoing applications are incorporated herein by reference.

Yet another embodiment of the invention relates to a method of producing a nematode-resistant transgenic plant, wherein the method comprises the steps of: a) transforming a wild-type plant with an expression vector comprising a polynucleotide encoding a; and c) selecting transgenic plants for increased nematode resistance.

A variety of methods for introducing polynucleotides into the genome of plants and for the regeneration of plants from plant tissues or plant cells are known in, for example, Plant Molecular Biology and Biotechnology (CRC Press, Boca Raton, Fla.), chapter 6/7, pp. 71-119 (1993); White FF (1993) Vectors for Gene Transfer in Higher Plants; Transgenic Plants, vol. 1, Engineering and Utilization, Ed.: Kung and Wu R, Academic Press, 15-38; Jenes Bet al. (1993) Techniques for Gene Transfer; Transgenic Plants, vol. 1, Engineering and Utilization, Ed.: Kung and R. Wu, Academic Press, pp. 128-143; Potrykus (1991) Annu Rev Plant Physiol Plant Molec Biol 42:205-225; Halford N G, Shewry P R (2000) Br Med Bull 56(1):62-73.

Transformation methods may include direct and indirect methods of transformation. Suitable direct methods include polyethylene glycol induced DNA uptake, liposome-mediated transformation (U.S. Pat. No. 4,536,475), biolistic methods using the gene gun (Fromm ME et al., Bio/Technology. 8(9):833-9, 1990; Gordon-Kamm et al. Plant Cell 2:603, 1990), electroporation, incubation of dry embryos in DNA-comprising solution, and microinjection. In the case of these direct transformation methods, the plasmids used need not meet any particular requirements. Simple plasmids, such as those of the pUC series, pBR322, M13 mp series, pACYC184 and the like can be used. If intact plants are to be regenerated from the transformed cells, an additional selectable marker gene is preferably located on the plasmid. The direct transformation techniques are equally suitable for dicotyledonous and monocotyledonous plants.

Transformation can also be carried out by bacterial infection by means of Agrobacterium (for example EP 0 116 718), viral infection by means of viral vectors (EP 0 067 553; U.S. Pat. No. 4,407,956; WO 95/34668; WO 93/03161) or by means of pollen (EP 0 270 356; WO 85/01856; U.S. Pat. No. 4,684,611). Agrobacterium based transformation techniques (especially for dicotyledonous plants) are well known in the art. The Agrobacterium strain (e.g., Agrobacterium tumefaciens or Agrobacterium rhizogenes) comprises a plasmid (Ti or Ri plasmid) and a T-DNA element which is transferred to the plant following infection with Agrobacterium. The T-DNA (transferred DNA) is integrated into the genome of the plant cell. The T-DNA may be localized on the Ri- or Ti-plasmid or is separately comprised in a so-called binary vector. Methods for the Agrobacterium-mediated transformation are described, for example, in Horsch RB et al. (1985) Science 225:1229. The Agrobacterium-mediated transformation is best suited to dicotyledonous plants but has also been adapted to monocotyledonous plants. The transformation of plants by Agrobacteria is described in, for example, White F F, Vectors for Gene Transfer in Higher Plants, Transgenic Plants, Vol. 1, Engineering and Utilization, edited by S. D. Kung and R. Wu, Academic Press, 1993, pp. 15-38; Jenes B et al. Techniques for Gene Transfer, Transgenic Plants, Vol. 1, Engineering and Utilization, edited by S. D. Kung and R. Wu, Academic Press, 1993, pp. 128-143; Potrykus (1991) Annu Rev Plant Physiol Plant Molec Biol 42:205-225.

The polynucleotides described herein can be directly transformed into the plastid genome. Plastid expression, in which genes are inserted by homologous recombination into the several thousand copies of the circular plastid genome present in each plant cell, takes advantage of the enormous copy number advantage over nuclear-expressed genes to permit high expression levels. In one embodiment, the nucleotides are inserted into a plastid targeting vector and transformed into the plastid genome of a desired plant host. Plants homoplasmic for plastid genomes containing the nucleotide sequences are obtained, and are preferentially capable of high expression of the nucleotides.

Plastid transformation technology is for example extensively described in U.S. Pat. Nos. 5,451,513, 5,545,817, 5,545,818, and 5,877,462 in WO 95/16783 and WO 97/32977, and in McBride et al. (1994) PNAS 91, 7301-7305.

The transgenic plants of the invention may be used in a method of controlling infestation of a crop by a plant nematode, which comprises the step of growing said crop from seeds comprising an expression vector comprising a promoter operably linked to a polynucleotide encoding at least one Annexin, AUX/IAA, Isoflavone 7-OMT, Anthocyanidin 3-glucoside rhamnosyltransferase-like, hsr201-like, Laccase, AP2-like, HI1 or TINY-like polypeptide, wherein the expression vector is stably integrated into the genomes of the seeds.

The invention is further illustrated by the following examples, which are not to be construed in any way as imposing limitations upon the scope thereof. Incorporated by reference is U.S. provisional patent application No. 61/236,624 filed 25, Aug. 2009.

### Example 1

### Vector Construction

Using available cDNA sequence for the soybean target polynucleotides, PCR was used to isolate DNA fragments used to construct the binary vectors described in Table 1 and discussed in Example 2. The PCR products were cloned into TOPO pCR2.1 vectors (Invitrogen, Carlsbad, Calif.), and inserts were confirmed by sequencing. Open reading frames described by the polynucleotides GmAnnAt4-like (SEQ ID NO:49), GmAux28 (SEQ ID NO:127), Gmlsoflavone70MT-9 (SEQ ID NO:117), GmAnUGT_{—}47218626 (SEQ ID NO:109), Gmhsr201-like (SEQ ID NO:105), MtTINY-like (SEQ ID NO:39), GmLaccase1 (SEQ ID NO:79) and GmHI1 (SEQ ID NO:21) were isolated using this method. Alternatively, available soybean genomic sequence was used to design primers for amplification of gene sequences from soybean genomic DNA to construct the binary vectors described in Table 1 and discussed in Example 2 and Example 3. DNA sequences for the soybean target genes were PCR amplified, cloned into TOPO pCR2.1 vectors (Invitrogen, Carlsbad, Calif.), and inserts were confirmed by sequencing. Gene fragments for the target polynucleotides GmAP2-like 1 (SEQ ID NO:1), GmAP2-like 2 (SEQ ID NO:3) and GmAP2-like 3 (SEQ ID NO:7) were isolated by PCR amplifying the polynucleotide sequences from soybean genomic DNA.

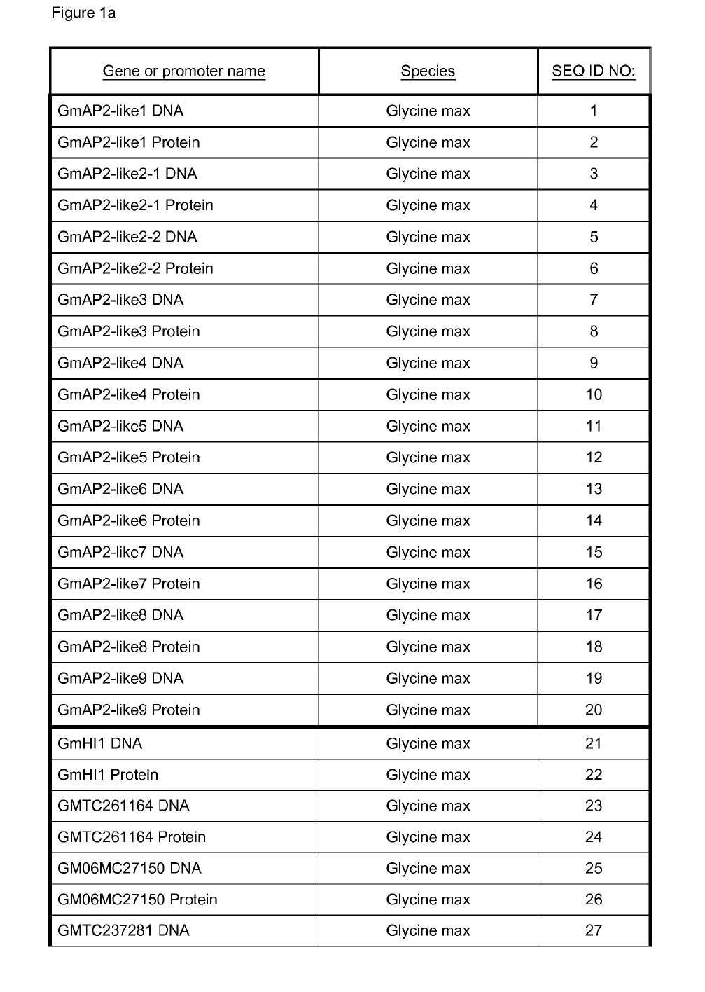

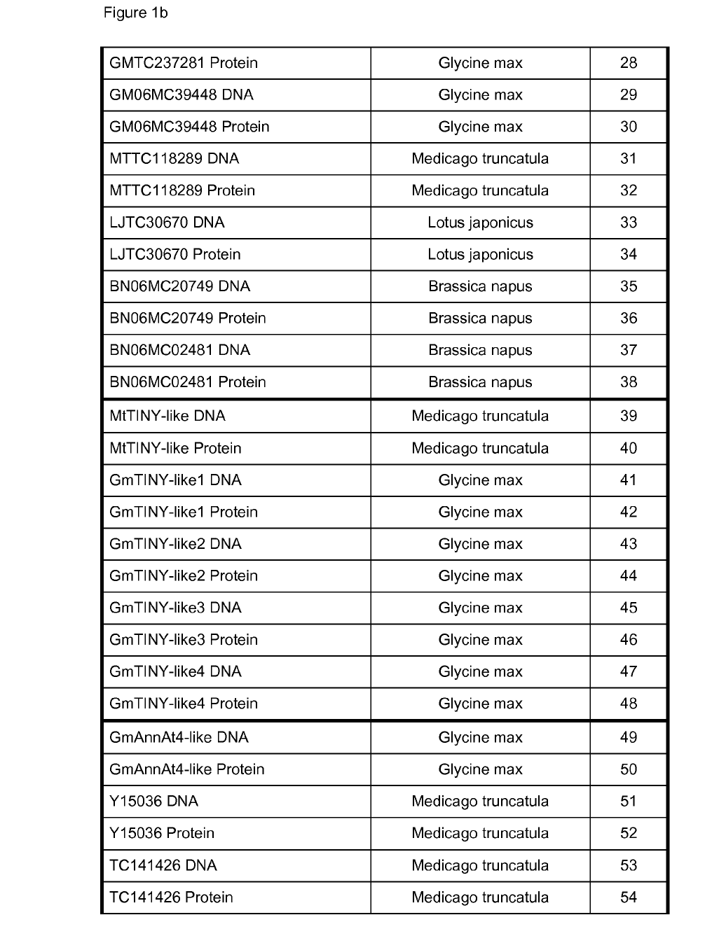

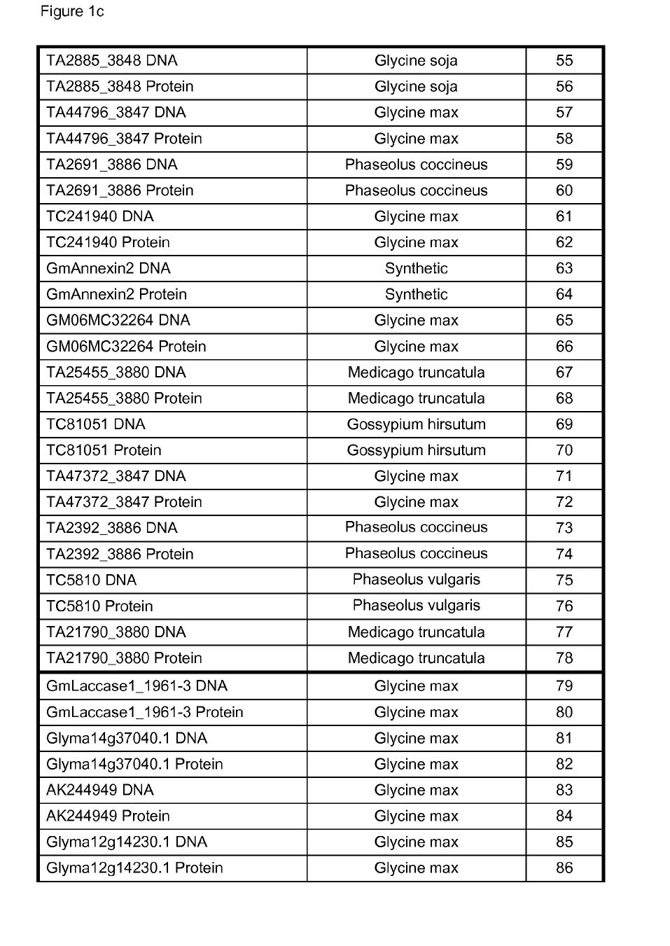

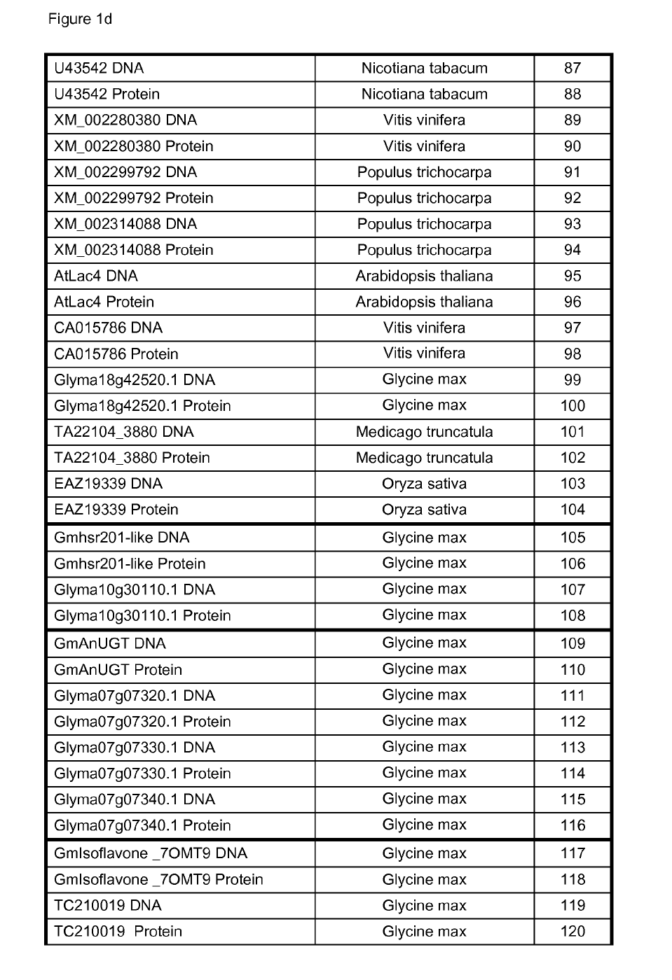

The cloned GmAnnAt4-like (SEQ ID NO: 49), GmAux28 (SEQ ID NO: 127), GmAnUGT_{—}47218626 (SEQ ID NO: 109), Gmhsr201-like (SEQ ID NO: 105) and GmLaccase1 (SEQ ID NO: 79) polynucleotides were sequenced and individually subcloned into a plant expression vector containing a TPP promoter from Arabidopsis thaliana designated p-AtTPP promoter (SEQ ID NO:135) in FIG. 1). The cloned Gmlsoflavone70MT-9 (SEQ ID NO:117) was sequenced and individually subcloned into a plant expression vector containing a Ubiquitin promoter from parsley (WO 03/102198; p-PcUbi4-2 promoter (SEQ ID NO:137) in FIG. 1). The cloned GmLaccase1 (SEQ ID NO: 79), MtTINY-like (SEQ ID NO: 39) polynucleotides were sequenced and individually subcloned into a plant expression vector containing an MtN3-like promoter from soybean designated p-MtN3-like (SEQ ID NO:136), also referred to as p-GmN3L, in FIG. 1, The cloned GmHI1 (SEQ ID NO:21), GmAP2-like1 (SEQ ID NO:1), GmAP2-like2 (SEQ ID NO:3) and GmAP2-like3 (SEQ ID NO:7) polynucleotides were sequenced and individually subcloned into a plant expression vector containing the SUPER promoter (U.S. Pat. No. 5,955,646) (SEQ ID NO:138 in FIG. 1). The selection marker for transformation was the mutated form of the acetohydroxy acid synthase (AHAS) selection gene (also referred to as AHAS2) from Arabidopsis thaliana (Sathasivan et al., Plant Phys. 97:1044-50, 1991), conferring resistance to the herbicide ARSENAL (Imazapyr, BASF Corporation, Mount Olive, N.J.). The expression of AHAS2 was driven by a ubiquitin promoter from parsley (WO 03/102198) (SEQ ID NO:137). Table 1 describes the constructs containing GmAnnAt4-like, GmAux28, Gmlsoflavone70MT-9, GmAnUGT_{—}47218626, Gmhsr201-like, GmLaccase1, GmAP2-like1, GmAP2-like2, GmAP2-like3, MtTINY-like and GmHI1 polynucleotides.

TABLE 1

| | Promoter | | |
|---|---|---|---|
| Vector Name | Name | Polynucleotide Name | SEQ ID NO: |
| RTP2833 | Super | GmAP2-like1 | 1 |
| RTP2834 | Super | GmAP2-like2 | 3 |
| RTP2839 | Super | GmAP2-like3 | 7 |
| RTP2766 | Super | GmHI1 | 21 |
| RBM056 | MtN3-like | MtTINY-like | 39 |
| RTP2424 | AtTPP | GmAnnAt4-like | 49 |
| RTP1960 | MtN3-like | GmLaccase1 | 79 |
| RTP1961 | AtTPP | GmLaccase1 | 79 |
| RTP1433 | AtTPP | Gmhsr201-like | 105 |
| MSB126 | AtTPP | GmAnUGT_47218626 | 109 |
| MSB131 | Ubi | GmIsoflavone 7OMT-9 | 117 |
| RTP1808 | AtTPP | GmAux28 | 127 |

### Example 2

### Nematode Bioassay

A bioassay to assess nematode resistance conferred by the polynucleotides described herein was performed using a rooted plant assay system disclosed in commonly owned copending U.S. Ser. No. 12/001,234. Transgenic roots were generated after transformation with the binary vectors described in Example 1. Multiple transgenic root lines were sub-cultured and inoculated with surface-decontaminated race 3 SCN second stage juveniles (J2) at the level of about 500 J2/well. Four weeks after nematode inoculation, the cyst number in each well was counted. For each transformation construct, the number of cysts per line was calculated to determine the average cyst count and standard error for the construct. The cyst count values for each transformation construct was compared to the cyst count values of an empty vector control tested in parallel to determine if the construct tested results in a reduction in cyst count. Rooted explant cultures transformed with vectors RTP2424, RTP1808, MSB131, MSB126, RTP1433, RTP1960, RTP1961, RTP2833, RTP2834, RTP2839, RBM056 and RTP2766 exhibited a general trend of reduced cyst numbers and female index relative to the known susceptible variety, Williams82.

Root area measurements were determined to evaluate the amount of root material for each subcultured line resulted from 4 weeks of growth after nematode inoculation. The root area values for each construct is compared to the root area values of an empty vector control tested in parallel to determine if the construct tested results in a change in root area. Rooted explant cultures transformed with vectors RTP2833, RTP2834, and RTP2839 exhibited a general trend of increased root area compared to an empty vector control.

### Example 3

### Root Biomass Assay

The rooted plant assay system disclosed in commonly owned copending U.S. Ser. No. 12/001,234 was also employed to assess root growth of uninfected transgenic roots comprising RTP2833, RTP2834, and RTP2839. Multiple transgenic root lines and connected cotyledon are sub-cultured to agar plates for observation. At the time of sub-culturing the root tip is marked on the back of plate as a point of reference. The sub-cultured root and cotyledon are incubated in a light chamber cotyledon side up for 6 days. For each transformation construct root weight, root length and number of root laterals is recorded. The root parameter measurement values for each transformation construct is compared to the root parameter measurement values of an empty vector control tested in parallel to determine if the construct tested results in a change in root weight, root length, root area, and root lateral number. Rooted explant cultures transformed with vectors RTP2833 and RTP2839 exhibited a general trend of increased root weight relative to the empty vector control.

1. A transgenic plant transformed with an expression vector comprising an isolated polynucleotide selected from the group consisting of: a) a polynucleotide encoding an AP2/EREBP transcription factor similar to SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, or SEQ ID NO:20; b) a polynucleotide encoding a harpin-induced protein similar to SEQ ID NO:22, SEQ ID NO:24, SEQ ID NO:26, SEQ ID NO:28, SEQ ID NO:30, SEQ ID NO:32, SEQ ID NO:34, SEQ ID NO:36, or SEQ ID NO:38; c) a polynucleotide encoding a TINY-like transcription factor similar to SEQ ID NO:40, SEQ ID NO:42, SEQ ID NO:44, SEQ ID NO:46, or SEQ ID NO:48; d) a polynucleotide encoding an annexin protein similar to SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, or SEQ ID NO:78; e) a polynucleotide encoding a laccase similar to SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, or SEQ ID NO:104; f) a polynucleotide encoding a benzoyl transferase similar to SEQ ID NO:106 or SEQ ID NO:108; g) a polynucleotide encoding a rhamnosyltransferase similar to SEQ ID NO:110, SEQ ID NO:112, SEQ ID NO:114, or SEQ ID NO:116; h) a polynucleotide encoding an isoflavone-7-O-methyltransferase similar to SEQ ID NO:118, SEQ ID NO:120, SEQ ID NO:122, SEQ ID NO:124, or SEQ ID NO:126; and i) a polynucleotide encoding an AUX/IAA protein similar to SEQ ID NO:128, SEQ ID NO:130, SEQ ID NO:132, or SEQ ID NO:134.

2. A seed which is true breeding for a transgene comprising at least one polynucleotide selected from the group consisting of: a) a polynucleotide encoding an AP2/EREBP transcription factor similar to SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, or SEQ ID NO:20; b) a polynucleotide encoding a harpin-induced protein similar to SEQ ID NO:22, SEQ ID NO:24, SEQ ID NO:26, SEQ ID NO:28, SEQ ID NO:30, SEQ ID NO:32, SEQ ID NO:34, SEQ ID NO:36, or SEQ ID NO:38; c) a polynucleotide encoding a TINY-like transcription factor similar to SEQ ID NO:40, SEQ ID NO:42, SEQ ID NO:44, SEQ ID NO:46, or SEQ ID NO:48; d) a polynucleotide encoding an annexin protein similar to SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, or SEQ ID NO:78; e) a polynucleotide encoding a laccase similar to SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, or SEQ ID NO:104; f) a polynucleotide encoding a benzoyl transferase similar to SEQ ID NO:106 or SEQ ID NO:108; g) a polynucleotide encoding a rhamnosyltransferase similar to SEQ ID NO:110, SEQ ID NO:112, SEQ ID NO:114, or SEQ ID NO:116; h) a polynucleotide encoding an isoflavone-7-O-methyltransferase similar to SEQ ID NO:118, SEQ ID NO:120, SEQ ID NO:122, SEQ ID NO:124, or SEQ ID NO:126; and i) a polynucleotide encoding an AUX/IAA protein similar to SEQ ID NO:128, SEQ ID NO:130, SEQ ID NO:132, or SEQ ID NO:134, wherein expression of the transgene confers increased nematode resistance to the plant grown from the transgenic seed.

3. An expression vector comprising a promoter operably linked to a polynucleotide encoding at least one polynucleotide selected from the group consisting of: a) a polynucleotide encoding an AP2/EREBP transcription factor similar to SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, or SEQ ID NO:20; b) a polynucleotide encoding a harpin-induced protein similar to SEQ ID NO:22, SEQ ID NO:24, SEQ ID NO:26, SEQ ID NO:28, SEQ ID NO:30, SEQ ID NO:32, SEQ ID NO:34, SEQ ID NO:36, or SEQ ID NO:38; c) a polynucleotide encoding a TINY-like transcription factor similar to SEQ ID NO:40, SEQ ID NO:42, SEQ ID NO:44, SEQ ID NO:46, or SEQ ID NO:48; d) a polynucleotide encoding an annexin protein similar to SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, or SEQ ID NO:78; e) a polynucleotide encoding a laccase similar to SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, or SEQ ID NO:104; f) a polynucleotide encoding a benzoyl transferase similar to SEQ ID NO:106 or SEQ ID NO:108; g) a polynucleotide encoding a rhamnosyltransferase similar to SEQ ID NO:110, SEQ ID NO:112, SEQ ID NO:114, or SEQ ID NO:116; h) a polynucleotide encoding an isoflavone-7-O-methyltransferase similar to SEQ ID NO:118, SEQ ID NO:120, SEQ ID NO:122, SEQ ID NO:124, or SEQ ID NO:126; and i) a polynucleotide encoding an AUX/IAA protein similar to SEQ ID NO:128, SEQ ID NO:130, SEQ ID NO:132, or SEQ ID NO:134.

4. A method of producing a nematode-resistant transgenic plant, wherein the method comprises the steps of: a) transforming a wild type plant cell with an expression vector comprising a promoter operably linked to a polynucleotide selected from the group consisting of: a) a polynucleotide encoding an AP2/EREBP transcription factor similar to SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, or SEQ ID NO:20; b) a polynucleotide encoding a harpin-induced protein similar to SEQ ID NO:22, SEQ ID NO:24, SEQ ID NO:26, SEQ ID NO:28, SEQ ID NO:30, SEQ ID NO:32, SEQ ID NO:34, SEQ ID NO:36, or SEQ ID NO:38; c) a polynucleotide encoding a TINY-like transcription factor similar to SEQ ID NO:40, SEQ ID NO:42, SEQ ID NO:44, SEQ ID NO:46, or SEQ ID NO:48; d) a polynucleotide encoding an annexin protein similar to SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, or SEQ ID NO:78; e) a polynucleotide encoding a laccase similar to SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, or SEQ ID NO:104; f) a polynucleotide encoding a benzoyl transferase similar to SEQ ID NO:106 or SEQ ID NO:108; g) a polynucleotide encoding a rhamnosyltransferase similar to SEQ ID NO:110, SEQ ID NO:112, SEQ ID NO:114, or SEQ ID NO:116; h) a polynucleotide encoding an isoflavone-7-O-methyltransferase similar to SEQ ID NO:118, SEQ ID NO:120, SEQ ID NO:122, SEQ ID NO:124, or SEQ ID NO:126; and i) a polynucleotide encoding an AUX/IAA protein similar to SEQ ID NO:128, SEQ ID NO:130, SEQ ID NO:132, or SEQ ID NO:134; b) regenerating transgenic plants from the transformed plant cell; and c) selecting transgenic plants for increased nematode resistance as compared to a control plant of the same species.

5. A method of increasing yield of a crop plant, the method comprising the steps of transforming a plant cell with an expression vector comprising a promoter operably linked to a polynucleotide encoding an AP2/EREBP transcription factor similar to SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, or SEQ ID NO:20; regenerating transgenic plants from the transformed plant cell, and selecting transgenic plants for increased root growth as compared to a control plant of the same species.


SEQUENCE LISTING (138 sequences)

SEQ ID NO: 1 - DNA, 1065 bp, Glycine max
atgtcaacctcgagaacctcatcagatacagccttcaaaggatatgacccgagccaaactcaaatgtgtctgtctctccttcaacgcaacacatctccttgtggtgagagaagaggtagaaggaagcaagcagaaccagggaggttccttggtgttaggaggcgcccttggggtcgatatgctgctgaaattagagaccccacaaccaaagagaggcattggcttggcacatttgacactgctcaagaagcagctcttgcttatgacagagctgctctgtccatgaaaggaagccaagctagaaccaactttgtttactctgacaacatcaacttccacactctccagctttctcctatgaatgttcaagttcaacctctcttgccagcttcacagacgttcttcaccaacaccaccaccaccaccactcagaccaaaacaccaaccaaccaaaatagcctctctcagattattcacagtgcttcaaactgtggaaacccctccccattgaacaatgacatgtgtgttgaaaccacatatgggtcagctcaggatgacaatttcttcttttctagtgattctaactcgggctatctggaatgcatagttcctgataactgcttcagacctgcttcatccagttccaacagttcaaactccagaaagagcaatgtgaatgatcaaaagactaacacaagctccatggagagtactaatcatcatcatcatcatcagttctcacatgttgatcacatgacttcgttctctcaagaagaaggcattaatgaaatggcaccaagggcctcttataacttatcagatttttgttatcaaagtgaagtgagtcaaggatcatgggattggaattgcagtgaactttcagctatatttaagaacccaatgagggtggaaaatgggtgcatggatgatgcattgtacccaataaatgatagtcctactagtccaagctatgggctaatgaatgaggctggttcttcttctacaacctgttctccatcacttccaccctttggggacgtagacttgggatacccactcttctga

SEQ ID NO: 2 - protein, 354 aa, Glycine max
MSTSRTSSDTAFKGYDPSQTQMCLSLLQRNTSPCGERRGRRKQAEPGRFLGVRRRPWGRYAAEIRDPTTKERHWLGTFDTAQEAALAYDRAALSMKGSQARTNFVYSDNINFHTLQLSPMNVQVQPLLPASQTFFTNTTTTTTQTKTPTNQNSLSQIIHSASNCGNPSPLNNDMCVETTYGSAQDDNFFFSSDSNSGYLECIVPDNCFRPASSSSNSSNSRKSNVNDQKTNTSSMESTNHHHHHQFSHVDHMTSFSQEEGINEMAPRASYNLSDFCYQSEVSQGSWDWNCSELSAIFKNPMRVENGCMDDALYPINDSPTSPSYGLMNEAGSSSTTCSPSLPPFGDVDLGYPLF

SEQ ID NO: 3 - DNA, 1116 bp, Glycine max
atgatgaagtcattgtctttaaaagacttcccacaacttcacccttttcattcatattctcttctatgctcccttaggtgtctatcactatttaacttccctttcccctcatcaaaaaacacacactttctttccattttccaagccatgtcaacctcaagaacctctgatgcttccctcaaaggatatgagtccaaccaaacccaaatgaatctgtccctccttcaacgtaacatgtctccttctggcgagagaagaggccgaggaaaacaagcagaacctggaaggtttctcggagtaagaaggcgcccttggggtagatatgctgctgaaatcagaaaccctttaactaaagaaagacattggcttggaacatttgacactgctcaagaagcagctcttgcttatgacagagctgctctgtccatgaaaggatgccaagcaagaaccaacttcatatattccaaagacaccaataccaacattttccacaatgctctcactcctatgaatactcaacctctcttgccaccttcacataacactcacaacacacaaccaaccaatcaaagtggcctatctcaccaccttggtgctatctcttcccacattgaaaacccttctgacatgagtgcatatgggtcacctcaggatgataatttcttcttctccaatgattctaactcgggctatcttgaatgcatagttcaggataactgcttcaggaagagcaatgccagtgcttcaagtcatgaccttacgattggccacgttaactcaaactccatgcatgttcagccacattttgacaccactcaagaagccttgaacatgcaaacaacagtgacttcaaactatgcagatatttcttaccctagtcaaggattatggaacaatcagcaatcatgggattgcagttccaatgaactagcagctatgtttaaaaatccatcaagggctgaggaagggtacatgtgcatgaatgcattaatgtatcccttcactgatagttcaagctacggttttatgactcaggctgaggctgtttcttcaactaccttttctccatcacttcctccctttggggatgctgatttgggatactcacccttttga

SEQ ID NO: 4 - protein, 371 aa, Glycine max
MMKSLSLKDFPQLHPFHSYSLLCSLRCLSLFNFPFPSSKNTHFLSIFQAMSTSRTSDASLKGYESNQTQMNLSLLQRNMSPSGERRGRGKQAEPGRFLGVRRRPWGRYAAEIRNPLTKERHWLGTFDTAQEAALAYDRAALSMKGCQARTNFIYSKDTNTNIFHNALTPMNTQPLLPPSHNTHNTQPTNQSGLSHHLGAISSHIENPSDMSAYGSPQDDNFFFSNDSNSGYLECIVQDNCFRKSNASASSHDLTIGHVNSNSMHVQPHFDTTQEALNMQTTVTSNYADISYPSQGLWNNQQSWDCSSNELAAMFKNPSRAEEGYMCMNALMYPFTDSSSYGFMTQAEAVSSTTFSPSLPPFGDADLGYSPF

SEQ ID NO: 5 - DNA, 855 bp, Glycine max
atgtcaacctcaagaacctctgatgcttccctcaaaggatatgagtccaaccaaacccaaatgaatctgtccctccttcaacgtaacatgtctccttctggcgagagaagaggccgaggaaaacaagcagaacctggaaggtttctcggagtaagaaggcgcccttggggtagatatgctgctgaaatcagaaaccctttaactaaagaaagacattggcttggaacatttgacactgctcaagaagcagctcttgcttatgacagagctgctctgtccatgaaaggatgccaagcaagaaccaacttcatatattccaaagacaccaataccaacattttccacaatgctctcactcctatgaatactcaacctctcttgccaccttcacataacactcacaacacacaaccaaccaatcaaagtggcctatctcaccaccttggtgctatctcttcccacattgaaaacccttctgacatgagtgcatatgggtcacctcaggatgataatttcttcttctccaatgattctaactcgggctatcttgaatgcatagttcaggataactgcttcaggaagagcaatgccagtgcttcaaatatttcttaccctagtcaaggattatggaacaatcagcaatcatgggattgcagttccaatgaactagcagctatgtttaaaaatccatcaagggctgaggaagggtacatgtgcatgaatgcattaatgtatcccttcactgatagttcaagctacggttttatgactcaggctgaggctgtttcttcaactaccttttctccatcacttcctccctttggggatgctgatttgggatactcacccttttga

SEQ ID NO: 6 - protein, 284 aa, Glycine max
MSTSRTSDASLKGYESNQTQMNLSLLQRNMSPSGERRGRGKQAEPGRFLGVRRRPWGRYAAEIRNPLTKERHWLGTFDTAQEAALAYDRAALSMKGCQARTNFIYSKDTNTNIFHNALTPMNTQPLLPPSHNTHNTQPTNQSGLSHHLGAISSHIENPSDMSAYGSPQDDNFFFSNDSNSGYLECIVQDNCFRKSNASASNISYPSQGLWNNQQSWDCSSNELAAMFKNPSRAEEGYMCMNALMYPFTDSSSYGFMTQAEAVSSTTFSPSLPPFGDADLGYSPF

SEQ ID NO: 7 - DNA, 852 bp, Glycine max
atgtcaacctcaagaacctcagatgcttccctcaaaggatatgagtccaaccaaacccaaatgaatttgtctctccttcaacgtaacatgtctccttctggtgagagaagaggccggggaaagcaagcagaacctggaaggtttctcggagtaagaaggcgcccttggggtagatatgctgctgaaatcagaaaccctttaactaaagaaagacattggcttggaacatttgacactgctcaagaagcagctcttgcttatgacagagctgctctgtccatgaaaggatgccaagcaagaaccaacttcatatattccaaagacaccaataccaacattttccacaatgctctcactcctatgaatgctcaacctctcttgccaccttcatataacactcacaacccacaaccaatcaatcaaagtggcctatctcaccttggtaacatttcttcccacattgaaaacccttctggcatgagttcatctgggtcacctcaggatgataatttcttcttctccagtgattctaactcgggctatcttgaatgcatagttcaggataactgttcaaggaagagcaatgccagtgcttcaagtcatgaccttaagcttggccacgttaatacaagttccatgcatgttcagccacattttgacaccactcatgaagtcctgaacatgcaaacaacagtggcttcaaactccgcagatttttcttaccctagtaaaggattatgggattgcagctccaatgaactagcagctatgtttaaaaatccatcaagggctgaggaagggtgcatgaatgcattgtatcccttcactgatagttcaagctacggttcatga

SEQ ID NO: 8 - protein, 283 aa, Glycine max
MSTSRTSDASLKGYESNQTQMNLSLLQRNMSPSGERRGRGKQAEPGRFLGVRRRPWGRYAAEIRNPLTKERHWLGTFDTAQEAALAYDRAALSMKGCQARTNFIYSKDTNTNIFHNALTPMNAQPLLPPSYNTHNPQPINQSGLSHLGNISSHIENPSGMSSSGSPQDDNFFFSSDSNSGYLECIVQDNCSRKSNASASSHDLKLGHVNTSSMHVQPHFDTTHEVLNMQTTVASNSADFSYPSKGLWDCSSNELAAMFKNPSRAEEGCMNALYPFTDSSSYGS

SEQ ID NO: 9 - DNA, 894 bp, Glycine max
atgtcaacctcgagaacctcatcagatacacccttcaaaggatatgatcctagccaaactcaaatgtgtctctctctcctccaacgcaacacttctccttgtggtgagaggaggggcagaaggaagcaagcagaaccagggaggttccttggtgtgaggaggcgcccttggggtcgatatgctgctgaaattagagaccccacaaccaaagagaggcattggcttggcacatttgacactgctcaagaagcagctcttgcttatgacagagctgctctgtccatgaaaggaagccaagctagaaccaactttgtttactctgataacatcaacttccacactctccagctttctcctatgaatgttcaagttcaacctctcttgccagcttcacagttcctcactaccaccaccactcagaccaaccaaaatagccactttcaggttattcacagtactccaaactgtggaaacccctccccattgaacaatgacatgtgtgttgaaaccacatatgggtcagctcaggatgataatttcttcttttccagtgattctaactcgggctatctcgaatgcatagttcctgatgattgcttcagacctgcttcatccagttccaacaaaggcattgaaatggcaccaagggcatcttataacttttcagatttttgttatcaaagtgaatcatgggattggaatagcagtgaactttcagctatatttaagaacccaatgagggtggaaaatgggtgcatggatgcattatgccccataaatgatagtcctagttcaagctatgggctaatgaatgaggctgcttcttcttctacaacctgttctccgtcacttccaccctttggggacgtagacttgggatacccactcttctga

SEQ ID NO: 10 - protein, 297 aa, Glycine max
MSTSRTSSDTPFKGYDPSQTQMCLSLLQRNTSPCGERRGRRKQAEPGRFLGVRRRPWGRYAAEIRDPTTKERHWLGTFDTAQEAALAYDRAALSMKGSQARTNFVYSDNINFHTLQLSPMNVQVQPLLPASQFLTTTTTQTNQNSHFQVIHSTPNCGNPSPLNNDMCVETTYGSAQDDNFFFSSDSNSGYLECIVPDDCFRPASSSSNKGIEMAPRASYNFSDFCYQSESWDWNSSELSAIFKNPMRVENGCMDALCPINDSPSSSYGLMNEAASSSTTCSPSLPPFGDVDLGYPLF

SEQ ID NO: 11 - DNA, 981 bp, Glycine max
atgaggcgtctcaacggggtagctccgattatcggacccgactcgaaaggcgacggtggactcatcgccaataacccgaaacggacctcggccgtgaacaagagggctttaagagaagacggcggcggtggtggcggcggcggagcgatgaggtaccgcggcgtgaggcgcaggccgtgggggcgttacgcggcggagataagggaccctcaatcgaaggagcggcgatggctgggaaccttcgacacggcggaggaagccgcttgcgcctacgactgcgctgctagagccatgaggggtctcaaagctcgcaccaacttcgtttacccaacttctccgcaaccttcttccgccaccaccgaacacttgttccctaacttcaacaacaacaacaactttcacaaacactcactcttcaatcaccacccccctcgaaacccttcttctctcaacatgcttctctttcgtgaccttattcactctaacccttctttgctttcttcttcttccactcagaacttccacgaccagttttacaacaagagtacttctactttttcatcgttacctgtttctccttctcttgctccccctagttattcgatgaataattcttgtggaggctctttgtctgttaagatgaacacgtttcccacttgtggaactaattttgctgaaaaaggtgatgatggtgatggttttttctctcgtgagtcttcggattcggggttgttggaggagatagttaacaagttcttgcctagaacgaagcctagtaaatgcgagactacttttgcaaatcctcaggaggagtcgcttcttctccctccgcttcaattcgacacctctaatgggtttaatagcatggctatggagaatgatcaaaacattattaataatgcagagaactgtgtcgttgaagatgtttttcagtaccaagagcttcttaatgctttcgcaatcagaatgcaaaatgcttaa

SEQ ID NO: 12 - protein, 326 aa, Glycine max
MRRLNGVAPIIGPDSKGDGGLIANNPKRTSAVNKRALREDGGGGGGGGAMRYRGVRRRPWGRYAAEIRDPQSKERRWLGTFDTAEEAACAYDCAARAMRGLKARTNFVYPTSPQPSSATTEHLFPNFNNNNNFHKHSLFNHHPPRNPSSLNMLLFRDLIHSNPSLLSSSSTQNFHDQFYNKSTSTFSSLPVSPSLAPPSYSMNNSCGGSLSVKMNTFPTCGTNFAEKGDDGDGFFSRESSDSGLLEEIVNKFLPRTKPSKCETTFANPQEESLLLPPLQFDTSNGFNSMAMENDQNIINNAENCVVEDVFQYQELLNAFAIRMQNA

SEQ ID NO: 13 - DNA, 825 bp, Glycine max
atgaaaccctacacgtcatcaccatcatcctccaagtccaaaaagaagccaacaacaacacaacaagatcatgaaacagcatggggaggaaggtaccttggagtgaggaggaggccatggggaaggtatgcggccgaaattcgcgacccttccactaaggaaagacattggttgggcacgtttgataccgcagaagaggccgccctagcatatgataaggcggcccgctccatgcggggctcacgtgctcgaaccaacttcatctatccggatactcctccgggttcttctgtcacctccatcctctctccagacgagcaaacccaaacccaaatccaccaggcccaagaagagctctcctctctattcaacccaaacccgtttccacaacctgacccgaacccgcaattttccttatgcgggttttcgggtatcccgaacactcctacattttctgccaccgaagaaattgcctcatccggtggctatagctacagttatggatacaccgaaggaggaaccggcgtggagagttcacattttaagctctttgatgatggtgaaacgcagcttccaccattgccaccggatatcacaagctccatgggctatgaaatgagtaatgggctttatggaaacgatgtcgggttttcggatccaagttgcggagctaatgcttccgggtcgggttacccgtattcggggttcgaatcaggtgattacgtggtgcatagtccactctttagtgcaatgccatcagtgtcagataatgtggcaacgggccatgagggttttgatttgggaagctcttcttatttcttctga

SEQ ID NO: 14 - protein, 274 aa, Glycine max
MKPYTSSPSSSKSKKKPTTTQQDHETAWGGRYLGVRRRPWGRYAAEIRDPSTKERHWLGTFDTAEEAALAYDKAARSMRGSRARTNFIYPDTPPGSSVTSILSPDEQTQTQIHQAQEELSSLFNPNPFPQPDPNPQFSLCGFSGIPNTPTFSATEEIASSGGYSYSYGYTEGGTGVESSHFKLFDDGETQLPPLPPDITSSMGYEMSNGLYGNDVGFSDPSCGANASGSGYPYSGFESGDYVVHSPLFSAMPSVSDNVATGHEGFDLGSSSYFF

SEQ ID NO: 15 - DNA, 408 bp, Glycine max
atggaaaacctttcaccattgatttacaaaaaccccattagaagaacttctaggcgatctacaatgtatcttggtgtgagaaaaaggccatggggaagatatgctgctgagattaggaacccatacaccaaagagagacactggctaggcacatttgacactgctgaagaggctgctatagcttatgatctttcatctatcaagatttgtggcattaatgctcgaactaattttcactacccttttgtgtctcttccaccacttcctatgtcgtcattgcctcctccaccgccaccgccgaccccagagttggatccaagtgttgaagtttgtctagagatgatgaatgctgcttcttacgatgtttttctaattctggctatggcctgcctcttatcaaatcaataa

SEQ ID NO: 16 - protein, 135 aa, Glycine max
MENLSPLIYKNPIRRTSRRSTMYLGVRKRPWGRYAAEIRNPYTKERHWLGTFDTAEEAAIAYDLSSIKICGINARTNFHYPFVSLPPLPMSSLPPPPPPPTPELDPSVEVCLEMMNAASYDVFLILAMACLLSNQ

SEQ ID NO: 17 - DNA, 882 bp, Glycine max
atggaggctagattcagtgagcagaaggtcgttgtcacttacaaacatgtcaaacctggcattttaaatgcagcagcaccaaaggtagttcggatttctgtgatggatccctatgccacagatgaggaagatgaagatgaagggttggttaatcatccaagaatgaagaagtttgtgaatgaaatcagaattgtggagaagaagagttcaactaacagtgagtctgcaggtgtaaatcgtactaatgtgtcactgagacagcattgcaagggaaaaactggagggagtctcgaagaaaagaagcttagaggaatccgtcaaaggccatggggaagatgggcagcagaaatcagagatcctgtgaagcgaagaagggtgtggttagggacttatgacaccgcggaagaagctgctatggtttatgacaaagcagctattacctttagaggctctaatgctcgcactaatttcatcaagcctccttcaagggatgttgttctcactaatgaaactgaagctagagtgactgtgagtggtgagaatgattcaagtgaagggtcgtctcattttccttcacctacttctgtgcttaaacttcaacctgttgaaattatgttggaggagttttctgaaacagatcttctaagcaaagaagagtctagttctcaagacagtttcttgtttttagattcacattcgccatccattgattgttatttcaattttgaaactcttccaccagtgtttcttggtgaaactagtgtaacacagcagcctgttttccatggcaacttcagtgacatttcccatcttgtcaatgaggattttcaatcatggaattgggatatagatagttacttcagtgatcctctttcgttatga

SEQ ID NO: 18 - protein, 293 aa, Glycine max
MEARFSEQKVVVTYKHVKPGILNAAAPKVVRISVMDPYATDEEDEDEGLVNHPRMKKFVNEIRIVEKKSSTNSESAGVNRTNVSLRQHCKGKTGGSLEEKKLRGIRQRPWGRWAAEIRDPVKRRRVWLGTYDTAEEAAMVYDKAAITFRGSNARTNFIKPPSRDVVLTNETEARVTVSGENDSSEGSSHFPSPTSVLKLQPVEIMLEEFSETDLLSKEESSSQDSFLFLDSHSPSIDCYFNFETLPPVFLGETSVTQQPVFHGNFSDISHLVNEDFQSWNWDIDSYFSDPLSL

SEQ ID NO: 19 - DNA, 819 bp, Glycine max
atgtcaacctcaagaacctcatcagatacacccttcaaaggatatgatcctagccaaactcaaatgtgtctttctctcctccaacgcaacacttctccttggggtgagaggagaggcagaaggaagcaagcagaaccagggaagttccttggtcttggcacatttgacactgctcaagaagcagctcttgcttatgacagggctgctctgtccatgaaaggaagccaagctagaacaacctttgtttactctaataacatcaacttccacactctccagctttctcctatgaatgttcaagttcaacctctcttgccagcttcacagttcctcactaataccaccatgactcagaccaaccaaaatagccactttcaggttattcatagtacttcaaactgtggaaacccctccccattgaacaatgacatatgtgttgaaaccacatatgggtcagctcaggatgataatttcttcttttccagtgattctaactcgggctatctcgaatacatagttcctgatgattgcttcagacctacttcatctagttccaacagttcaaactctagaaagagcaacatgagtgatcaaaaggctaacacaagctccatggagagtactaatcaccatcatgatcagtactcatatgttgacatgacttcattctctcaagaaggcattgaaatggcaccaaggacaacttataactttttagatttttgttatgaaagtgaagtgagtcaaggatcatgggatgatactactagaaaaaaggccttgattgttgttgttgtttgtatgttagatgttttctaa

SEQ ID NO: 20 - protein, 272 aa, Glycine max
MSTSRTSSDTPFKGYDPSQTQMCLSLLQRNTSPWGERRGRRKQAEPGKFLGLGTFDTAQEAALAYDRAALSMKGSQARTTFVYSNNINFHTLQLSPMNVQVQPLLPASQFLTNTTMTQTNQNSHFQVIHSTSNCGNPSPLNNDICVETTYGSAQDDNFFFSSDSNSGYLEYIVPDDCFRPTSSSSNSSNSRKSNMSDQKANTSSMESTNHHHDQYSYVDMTSFSQEGIEMAPRTTYNFLDFCYESEVSQGSWDDTTRKKALIVVVVCMLDVF

SEQ ID NO: 21 - DNA, 630 bp, Glycine max
atgtcggtaaaggagtgtcaccaccacaagggaaagaagcacaaactctggagacgcatcttctggggcatagtgatcttcgccttcatcgtgttgctaacagttcttataatctgggcaatcctaaagccttcaaagccaaccttcatcctccaagacgtgaccgtctacggcttcaacgccaccatccccaacttcctcacctcaagctttcaggtcacgctttcctcgcgcaaccccaacgacaaaatcggagtctactacgaccgtctcgacacctacgtcacttaccggaaccagcaggtcacgtaccgaacctcaatcccaccctcataccaaggtcacaaggaagaggatgtgtggtccccattcgtcttcggcactaacgtccccgtcgcacccttcaacttcgttggtctcagtcaagaccagaccaacggcaacgtcctcgtcctcgttaagattgacggtaaggtacgatggaaagttggtaccttcgtctctggtcactacaacctctatgtaaggtgccctgctttcataacttttggcccccaaagcaccgggattgcccttggcaagaacgccgttaagtatcaattggttcaacgctgcaccgtcggggtttga

SEQ ID NO: 22 - protein, 209 aa, Glycine max
MSVKECHHHKGKKHKLWRRIFWGIVIFAFIVLLTVLIIWAILKPSKPTFILQDVTVYGFNATIPNFLTSSFQVTLSSRNPNDKIGVYYDRLDTYVTYRNQQVTYRTSIPPSYQGHKEEDVWSPFVFGTNVPVAPFNFVGLSQDQTNGNVLVLVKIDGKVRWKVGTFVSGHYNLYVRCPAFITFGPQSTGIALGKNAVKYQLVQRCTVGV

SEQ ID NO: 23 - DNA, 633 bp, Glycine max
atgtcggttaaggagtgccaccaccaccacaagggaaagaagcacaagctctggagacgcatcttctggggaatagtgatcttcgccttcatcgtgttactgacagttcttataatctgggcaatcctaaagccttcaaaaccaaccttcatcctccaagacgtcaccgtttacggcttcaatgccaccatccccaacttcctcacctcaagttttcaggtcacgctttcctcgcgcaaccccaacgacaaaatcggaatctactacgaccgtctcaacacctacgtaacttaccggagccagcaggtcacgtaccgaacctcaatcccaccctcataccaaggtcacaaggaagaggatgtgtggtccccattcgtcttcggcactaacgtccccgtcgcacccttcaacttcgttggtctcagtcaagaccagaccaacggcaacgtcctcgtcctcgttaagattgacggtaaggtacgatggaaagttggtaccttcgtctctggtcactacaacctctatgtaaggtgccctgctttcataacttttggcccccaaagcaccgggattgcccttggcaagaacgccgttaagtatcaattggttcaacgctgcaccgtcggggtttga

SEQ ID NO: 24 - protein, 210 aa, Glycine max
MSVKECHHHHKGKKHKLWRRIFWGIVIFAFIVLLTVLIIWAILKPSKPTFILQDVTVYGFNATIPNFLTSSFQVTLSSRNPNDKIGIYYDRLNTYVTYRSQQVTYRTSIPPSYQGHKEEDVWSPFVFGTNVPVAPFNFVGLSQDQTNGNVLVLVKIDGKVRWKVGTFVSGHYNLYVRCPAFITFGPQSTGIALGKNAVKYQLVQRCTVGV

SEQ ID NO: 25 - DNA, 633 bp, Glycine max
atgtcggttaaggagtgccaccaccaccacaagggaaagaagcacaagctctggagacgcatcttctggggaatagtgatcttcgccttcatcgtgttactgacagttcttataatctgggcaatcctaaagccttcaaaaccaaccttcatcctccaagacgtcaccgtttacggcttcaatgccaccatccccaacttcctcacctcaagttttcaggtcacgctttcctcgcgcaaccccaacgacaaaatcggaatctactacgaccgtctcaacacctacgtaacttaccggaaccagcaggtcacgtaccgaacctcaatcccaccctcgtaccaaggtcacaaggaagaggatgtgtggtccccgtttgtctacggcactaatgtccccgtcgcaccctacaacttcgttggcctcagtcaagaccagaccaacggcaacgtcctcgtcctcgttaagattgacggtaaggtacgatggaaagttggttccttcgtctctgctcactacaacctcaatgtaaggtgccctgctttcataacctttggcccccaaagcaacgggattgcccttggcaataacgccgttaagtatcaattggttcaacgctgcaccgtcggggtttga

SEQ ID NO: 26 - protein, 210 aa, Glycine max
MSVKECHHHHKGKKHKLWRRIFWGIVIFAFIVLLTVLIIWAILKPSKPTFILQDVTVYGFNATIPNFLTSSFQVTLSSRNPNDKIGIYYDRLNTYVTYRNQQVTYRTSIPPSYQGHKEEDVWSPFVYGTNVPVAPYNFVGLSQDQTNGNVLVLVKIDGKVRWKVGSFVSAHYNLNVRCPAFITFGPQSNGIALGNNAVKYQLVQRCTVGV

SEQ ID NO: 27 - DNA, 627 bp, Glycine max
atgtcggttagggagtgccaccaccaccccaagggaaagaagcacaagctctggagacgcatcttctggggaatagtgatcttcgccttcatcgtgttactgacagttcttataatctgggcaatcctaaagccttcaaaaccaaccttcatcctccaagacgtcaccgtttacggcttcaatgccaccatccccaacttcctcacctcaagttttcaggtcacgctttcctcgcgcaaccccaacgacaaaatcggaatctactacgaccgtctcaacacctacgtaacttaccggaaccagcaggtcacgtaccgaacctcaatcccaccctcgtaccaaggtcacaaggaagaggatgtgtggtccccgtttgtcactaatgtccccgtcgcaccctacaacttcgttggcctcagtcaagaccagaccaacggcaacgtcctcgtcctcgttaagattgacggtaaggtacgatggaaagttggttccttcgtctctgctcactacaacctcaatgtaaggtgccctgctttcataacctttggcccccaaagcaacgggattgcccttggcaataacgccgttaagtatcaattggttcaacgctgcaccgtcggggtttga

SEQ ID NO: 28 - protein, 208 aa, Glycine max
MSVRECHHHPKGKKHKLWRRIFWGIVIFAFIVLLTVLIIWAILKPSKPTFILQDVTVYGFNATIPNFLTSSFQVTLSSRNPNDKIGIYYDRLNTYVTYRNQQVTYRTSIPPSYQGHKEEDVWSPFVTNVPVAPYNFVGLSQDQTNGNVLVLVKIDGKVRWKVGSFVSAHYNLNVRCPAFITFGPQSNGIALGNNAVKYQLVQRCTVGV

SEQ ID NO: 29 - DNA, 630 bp, Glycine max
atgtcggtgaaggagtgcgagcaccacaaggggaagaagaggaagatcttccggcaagtgttctggtgcttagtggtgttccttttcatcgtgctggtcacaattcttttgatatgggcaatccttagacccaccaaacccaccttcacgctccaagacgtcaccgtctacgccttcaacgccaccgtggcgaacttcctcacttcgaattttcaggtcacgctcatctcgcgtaacccgaacgaccgcatcggtgtttactatgatcgcctcgaaaccttcgtgacctaccggagccagcaggtcacgtaccgaaccgccatccctccgacatatcaaggccataaagagatcaatgtttggtctccgtttgtttatggcaccaacatccccgtcgcgccgttcaatttcctcggcctcagccaggaccagagtaacggtaacgtcctcgtcaccatccgagccgagggtagggttcgttggaaggtcggcaccttcatctccggccgctaccacctctacgtccgctgcccggcgttcatcagctttggaccccgcagcaatggaatcgtcgtcggagagaacgccatcaagttccagattatccaacggtgctccgttagtgtctaa

SEQ ID NO: 30 - protein, 209 aa, Glycine max
MSVKECEHHKGKKRKIFRQVFWCLVVFLFIVLVTILLIWAILRPTKPTFTLQDVTVYAFNATVANFLTSNFQVTLISRNPNDRIGVYYDRLETFVTYRSQQVTYRTAIPPTYQGHKEINVWSPFVYGTNIPVAPFNFLGLSQDQSNGNVLVTIRAEGRVRWKVGTFISGRYHLYVRCPAFISFGPRSNGIVVGENAIKFQIIQRCSVSV

SEQ ID NO: 31 - DNA, 633 bp, Medicago truncatula
atgtcggtaaaagagtgcaaccaccacaaaggaaagaaacacaaacgcttcagaaaaatcttctggggaatcataatcttcctcttcattgtcctagtaacaatcctaatcatttgggctgtcttaaaaccttctaaaccttctttcattctccaagatgtcactgtttacgctttcaacgccacagttccaaatttcttaacctcaaatttccaagtcacagtttcttcacgtaatcccaacgataaaattggtatctactacgatcgtcttgattcttacgtcacataccggagccaacaaattacttacagaaccgccatacctccgtcgtatcagggacacaaagaggttgatgtttggtctccgtttgtttacggaacaaacgttccggtagctccatataactctgttgctcttagccaagaccaggataacggtaacgttcttgttgtcgttaaatttgatggtagggttaggtggaaggttggtgcttttatttctggtcattaccatatttttgttcgttgtcctgcttttatcacgtttggtccaagaagtaacggtattagcgtcggtgatagcggcgctgttaagtatcagattgttcaacggtgcactgtcagtgtttga

SEQ ID NO: 32 - protein, 210 aa, Medicago truncatula
MSVKECNHHKGKKHKRFRKIFWGIIIFLFIVLVTILIIWAVLKPSKPSFILQDVTVYAFNATVPNFLTSNFQVTVSSRNPNDKIGIYYDRLDSYVTYRSQQITYRTAIPPSYQGHKEVDVWSPFVYGTNVPVAPYNSVALSQDQDNGNVLVVVKFDGRVRWKVGAFISGHYHIFVRCPAFITFGPRSNGISVGDSGAVKYQIVQRCTVSV

SEQ ID NO: 33 - DNA, 633 bp, Lotus japonicus
atgtcggtgaagacgtgcgaccaccacaaggggaagaagaagaaaatcttccggcgaatcttctggggcatcgtgatcttcctcttcatcgtcctcgtaacaatcctaatcatctgggcaatcctgaagcccaataaacccaccttcacactccaagacgtaaccgtctacgccttcaacgccacaatggccaacttcctcacctccaacttccaagtcacgctctccgcccgcaaccctaacgacaagatcggcgtctactacgaccgccttgacgcctacatcacctaccagagccagcaagtcacctaccgcaccgccatccctccgtcgtaccagggccacaaggagatggacgtttggtccccgtttgtatacggcaccaacgtcccggtggctccctacaattttgttactctaagccaggaccaggccaacggtaacgtcctcgtcaccgttaaagttgacggcaaggttcgatggaaggtcggcgcttttgtcaccggccactaccacctcaacgtccggtgccctgcttttattaccttcggcgccagaagcaacggcgtcgccgttggtgatagcggcgccgttaagtatcagctcgtccagcgctgctccgtcagcgtctga

SEQ ID NO: 34 - protein, 210 aa, Lotus japonicus
MSVKTCDHHKGKKKKIFRRIFWGIVIFLFIVLVTILIIWAILKPNKPTFTLQDVTVYAFNATMANFLTSNFQVTLSARNPNDKIGVYYDRLDAYITYQSQQVTYRTAIPPSYQGHKEMDVWSPFVYGTNVPVAPYNFVTLSQDQANGNVLVTVKVDGKVRWKVGAFVTGHYHLNVRCPAFITFGARSNGVAVGDSGAVKYQLVQRCSVSV

SEQ ID NO: 35 - DNA, 639 bp, Brassica napus
atgaccttcaaagactgtggctcccacactcactctcgccggaaactaatccggcggcgaataatatggtcaataatcttcatcctcttcatcatctgcctcacaatccttctcatttgggcaatcctccaaccttcaaagccacgtttcatcctccaagacgccaccgtctacgccttcaacgtctccggcaatccaccgaacctcctcacctccaacttccaaatcactctttcttcccggaaccctaacgacaagatcggcatctactacgaccgtcttgacgtctacgctacttaccatagtcaacagatcactttcagaacatcgatccctccgacgtatcagggacacaaagaagtcaacatatggtcgccgtttgtctacgggacctccgttcccgtcgctccgttcaacgccgttagtcttgacggtgatcaagataacggcgctgttacgctgatcatccgtgctgacggtagagtgaggtggaaagttgggacgtttattacagggaagtatcatctttacgtgaggtgtcttgcgtatattaacttcggtaataaagctgccggagttattgtcggagataacgccgttaagtatacgcttacgagtagttgtagtgttagtgtctaa

SEQ ID NO: 36 - protein, 212 aa, Brassica napus
MTFKDCGSHTHSRRKLIRRRIIWSIIFILFIICLTILLIWAILQPSKPRFILQDATVYAFNVSGNPPNLLTSNFQITLSSRNPNDKIGIYYDRLDVYATYHSQQITFRTSIPPTYQGHKEVNIWSPFVYGTSVPVAPFNAVSLDGDQDNGAVTLIIRADGRVRWKVGTFITGKYHLYVRCLAYINFGNKAAGVIVGDNAVKYTLTSSCSVSV

SEQ ID NO: 37 - DNA, 639 bp, Brassica napus
atgacctacaaagactgtggctcccatagtcactctcgccggaaactaatccgtcgcataatatggtcaataatcatcatcctcttcataatcttcctcacaatactcctcatttgggcaattctccaaccttcaaagccacgtttcatcctccaagacgccaccgtctacgccttcaacgtctccggtaatccaccgaacctcctcacctccaacttccaagtcactctctcttcccggaaccctaacgacaagatcggcgtttactacgaccgtctcgaggtatacgctgcttaccagaaccagcagatcacttaccgaacatcgatccctcctacgtaccaaggacacaaagaagtcaacatctggtcgccgtttgtctacggaaactccgtccccatcgctccttttaacggcgttagtcttgacggcgatcaagaaaacggcgtcattaggttggatattcgtgctgacggcagagtgaggtggaaggttggggcgtttatcacagggaagtatcatctttatgtgaggtgtcaggcgtttattaacttcgctaataaagctgctgccggagttattgtcggagataacgccgttaagtatacgtatgctcagggttgtagtgttagtgtctaa

SEQ ID NO: 38 - protein, 212 aa, Brassica napus
MTYKDCGSHSHSRRKLIRRIIWSIIIILFIIFLTILLIWAILQPSKPRFILQDATVYAFNVSGNPPNLLTSNFQVTLSSRNPNDKIGVYYDRLEVYAAYQNQQITYRTSIPPTYQGHKEVNIWSPFVYGNSVPIAPFNGVSLDGDQENGVIRLDIRADGRVRWKVGAFITGKYHLYVRCQAFINFANKAAAGVIVGDNAVKYTYAQGCSVSV

SEQ ID NO: 39 - DNA, 621 bp, Medicago truncatula
atgaaagaaataaaaaactatttctcaccttcaaatccaaactcttcaaaacctaaaaaaacagagacaaagaaacagagcaaggtgaagaaaaacagagactgcaacagccacccggtttatcacggcgttcgaatgcgaagctggggaaaatgggtatcggaaatccgcgaacctcgcaaaaaatcacgaatctggcttggaacttacgctactcctgaaatggcagctagagcgcatgatgttgctgctttgagtataaagggtcattcagcaattcttaacttccccgaaatagcgcacatgcttccaagaccaacaacgttgtctccacgtgacattcaagttgcagctgcagaagctgctgccatggtggaatttgattctcaaatattgcgctcaacaatctcactcagctctgaagtctctgactcaccttcatattcatcagagtctacagagcttagtgagattgttgagcttcctaatatagaagagagttataactcggttgaggtgagaagtgagtttatgttgattgactcggttgaaagttggttgtatccgtcagtagaaagtttatgtgacatgatggttgaagaacagagttttttgttataa

SEQ ID NO: 40 - protein, 206 aa, Medicago truncatula
MKEIKNYFSPSNPNSSKPKKTETKKQSKVKKNRDCNSHPVYHGVRMRSWGKWVSEIREPRKKSRIWLGTYATPEMAARAHDVAALSIKGHSAILNFPEIAHMLPRPTTLSPRDIQVAAAEAAAMVEFDSQILRSTISLSSEVSDSPSYSSESTELSEIVELPNIEESYNSVEVRSEFMLIDSVESWLYPSVESLCDMMVEEQSFLL

SEQ ID NO: 41 - DNA, 636 bp, Glycine max
atgactgaaacagcaaggtgtcacgaaactccaagcagcagcaccagcaactcctccacctcaaaacccctgaaaacagagaccccgaaacagagcaaagcaaagagaaacagagaccccaccaagcattccgattaccacggcgtgcgaatgcgcaattggggcaaatgggtatccgaaattcgcgagcctcgcaagaaatcacgcatctggctcggaactttcgccacccctgaaatggcggcacgtgcgcacgatgttgcggctttgagcattaagggccacacggcggttctcaactttccaaacatagtaaacatgcttcccagacccgtcacgtgtgccccacgtgacattcaagccgcggccgccgaagccgcggcgatggtggagttcgaccgggagagctcggaggcggagtcgacggagctgagcgagattgtggagcttccgaacattgaagagagcttcgactcggttgagtcgagaagtgaattcgcgttgatcgactcggtggatagttgggtgtaccctcctccaatgacagtggggagcatttgtgacatgatgatgcttggggaacatgagtgtggattgttccaatttggaaccgatcaccaaacatgttctgtttag

SEQ ID NO: 42 - protein, 211 aa, Glycine max
MTETARCHETPSSSTSNSSTSKPLKTETPKQSKAKRNRDPTKHSDYHGVRMRNWGKWVSEIREPRKKSRIWLGTFATPEMAARAHDVAALSIKGHTAVLNFPNIVNMLPRPVTCAPRDIQAAAAEAAAMVEFDRESSEAESTELSEIVELPNIEESFDSVESRSEFALIDSVDSWVYPPPMTVGSICDMMMLGEHECGLFQFGTDHQTCSV

SEQ ID NO: 43 - DNA, 648 bp, Glycine max
atgaccgaaacaacaaggtgtcacgaaactcctagcagcagcaccagcaccagcaactcttccacttcaaaacccccgaaacggaacaagacagaaaaacagagcaaagcaaagagaaacagagaccccaccaagcatcccgattaccacggcgttcgaatgcgcaattggggcaaatgggtatcagaaattcgcgagccgcgcaagaaatcacgcatctggctcggaacttttgccacccctgaaatggcggcacgtgcacacgacgtggcggctttgagcataaaaggccacaccgcgattctcaacttccctaacatagtaaacatgcttcccagacccgtcacgtgtgccccacgtgacatccaagccgcggccgcggaagccgccgcgatggtggagttcgaccgtgagggttcggaggcagagtcgtcggagctgagcgagattgtggagcttccgaacatagaagagagcttcgagtcggttgagtcgagaagtgagttcacgttgatcgactcggtagatagttgggtgtaccctcctccaatggcggtggggagcattggtgacatgatgatgcttggggaacacgagtgtggattattccaatttgcgaccgatcaccaaaaaacatgttccgtttag

SEQ ID NO: 44 - protein, 215 aa, Glycine max
MTETTRCHETPSSSTSTSNSSTSKPPKRNKTEKQSKAKRNRDPTKHPDYHGVRMRNWGKWVSEIREPRKKSRIWLGTFATPEMAARAHDVAALSIKGHTAILNFPNIVNMLPRPVTCAPRDIQAAAAEAAAMVEFDREGSEAESSELSEIVELPNIEESFESVESRSEFTLIDSVDSWVYPPPMAVGSIGDMMMLGEHECGLFQFATDHQKTCSV

SEQ ID NO: 45 - DNA, 780 bp, Glycine max
atgacacaacaaacaacaacctcttcctcctcacattcgggcccaagggcccaggcccagaaacagagcaagaggcccagggactgcagcaagcacccggtgtaccacggcgtgcggaagcggaactggggcaaatgggtgtccgaaatccgggagccacgcaagaagtcccgaatctggctcggaacattctccactcccgaaatggcggcgcgagcccacgacgtggcggccctcaccatcaagggccagtcagcaatcctcaacttccccgaaattgcagacctgctccctaggcccgtcacgtgctccccacgtgacatccagaccgcggccacggccgcggcctccatggttaagttcgaccctgtgacacaatcctcagactcggagactccagagtcttcggagctgagcgagattgtcgaacttcccaacattgaagacagcagcgttgactcgacaccggagttcgtcttggtcgatgttgttgacagttgggtgtttcccccaatgggaatggagttttattggttccgagatagagatactacccatttgggattgaatggtgagtgggattcatgtgccttttgctcgcttcacgtgggattggcaaatactagcattcatggctgttttgctgtcaatggtccagggccacttcaatttactctctactgtgtctgtgtgcactatatttttcacactatatacatttgtaaatcactaaattgggatgatatatattcacagaggggaatcaccgattgcttctag

SEQ ID NO: 46 - protein, 259 aa, Glycine max
MTQQTTTSSSSHSGPRAQAQKQSKRPRDCSKHPVYHGVRKRNWGKWVSEIREPRKKSRIWLGTFSTPEMAARAHDVAALTIKGQSAILNFPEIADLLPRPVTCSPRDIQTAATAAASMVKFDPVTQSSDSETPESSELSEIVELPNIEDSSVDSTPEFVLVDVVDSWVFPPMGMEFYWFRDRDTTHLGLNGEWDSCAFCSLHVGLANTSIHGCFAVNGPGPLQFTLYCVCVHYIFHTIYICKSLNWDDIYSQRGITDCF

SEQ ID NO: 47 - DNA, 756 bp, Glycine max
atgtgcctggggctccaagaaaccaaaaacattcccaccattcttaacttcactcacttacaaatcacaatgacacaacaagcaacaacctcttccgaaacaacaacttcctcttcttcttcttcttcctcctcctcctccacattttcaccctcgcagcattcgggcccaaaggcccaggcccagaaacagagcaagaggcccagggactgcagcaagcacccggtgtaccacggcgtgcggaagcgcaactggggcaaatgggtgtccgaaatccgcgagccacgcaagaagtcccgaatctggctcggaacattcgccactcctgaaatggcggcgcgagcccacgacgtggcggctctcaccatcaagggcgagtcagcaatcctcaatttccccgaaattgcggacctgctccccaggcccgtcacgtgcgctccacgtgacatccaggctgcggccacggccgcggcctccatggttaaattcgaccccgtgacgcaatcctcggactcggagactccggagtcttcggagctgagcgagattgttgaacttcccaacattgaagacagcagctttgactccgttgactccagaccggagttcgtgttggtcgatgttgttgacggttgggtgtttccgccaatgggaatggggtcagagggaatcgagatctgtgccgctttttctgatgagttgtttccgcaacagagttttgttgatgagatagagatactacccatttgggattga

SEQ ID NO: 48 - protein, 251 aa, Glycine max
MCLGLQETKNIPTILNFTHLQITMTQQATTSSETTTSSSSSSSSSSSTFSPSQHSGPKAQAQKQSKRPRDCSKHPVYHGVRKRNWGKWVSEIREPRKKSRIWLGTFATPEMAARAHDVAALTIKGESAILNFPEIADLLPRPVTCAPRDIQAAATAAASMVKFDPVTQSSDSETPESSELSEIVELPNIEDSSFDSVDSRPEFVLVDVVDGWVFPPMGMGSEGIEICAAFSDELFPQQSFVDEIEILPIWD

SEQ ID NO: 49 - DNA, 945 bp, Glycine max
atggctaccctcattgctcccatcaccttttcgcctggtttagatgcagaagctcttagaaaagctttccaaggatgggggactgatgagaagaccgttatagcaatattgggtcacagaaatgttcatcagaggcaacaaatcagaaagatttatgaggaaatttaccaagaggacctcatcaaacgcctagagtctgagctctctggtgactttgagagagctgtgtaccgttggatgctggaacctgcagatcgtgatgctgttttggccaatgttgccatcaagaatggcagcaaaggttaccatgtgattgtggaaattgcttgtgtgctttcagctgaagaggtcttggcagtgaagcgtgcctatcacaaccgttacaagagatctttggaagaagatgtagctactaataccactggtgacattcgccagctgttggttgggttggtgaccgcatataggtatggtggagatgagatcaatgcaaaattggccaaaactgaagctgatattcttcatgaatctatcaaggagaaaaagggcaaccatgaagaagccatcaggatccttactacaaggagcaagacccaacttctggcaactttcaaccgctacagagatgaccatggcgcttctataactaagaaattgttggataatgcatccactgactttcagaaggcattgcacactgctattagatgtatcaatgaccacaaaaagtactatgaaaaggttctgcgcaacgctctaaaaaatgttggaactgatgaggatgcactgacccgtgtggtggtctcaagggctgagaaggacctcagggacatcaaagagcgatattataagagaaacagtgttcaccttgaggatgcagtggccaaggaaatctcaggggactacaagaaattcatcctcactctgttggggaaggaagattga

SEQ ID NO: 50 - protein, 314 aa, Glycine max
MATLIAPITFSPGLDAEALRKAFQGWGTDEKTVIAILGHRNVHQRQQIRKIYEEIYQEDLIKRLESELSGDFERAVYRWMLEPADRDAVLANVAIKNGSKGYHVIVEIACVLSAEEVLAVKRAYHNRYKRSLEEDVATNTTGDIRQLLVGLVTAYRYGGDEINAKLAKTEADILHESIKEKKGNHEEAIRILTTRSKTQLLATFNRYRDDHGASITKKLLDNASTDFQKALHTAIRCINDHKKYYEKVLRNALKNVGTDEDALTRVVVSRAEKDLRDIKERYYKRNSVHLEDAVAKEISGDYKKFILTLLGKED

SEQ ID NO: 51 - DNA, 942 bp, Medicago truncatula
atggctaccctttctgctcctagcaaccattctcccaatgaagatgctgaagctctaagaaaggctttcgaaggatggggtactgatgagaagacagtgataacaatacttggtcatagaaactctaatcagatacagcaaatcagaaaagcttatgaaggcatttacaatgaggatcttatcaaacgattggaatctgagatcaaaggagactttgagaaagctgtgtaccgttggattcttgaacctgcggagcgtgatgctgttttggcaaatgttgctatcaagagtggcaaaaattacaatgtcattgtggaaatttctgctgttctctcccccgaagagctcttgaatgtgagacgtgcttacgtcaaacgctacaagcactccttggaagaagatttggctgcccatacttccggccatcttcgccagcttttggtagggctagtgactgcatttaggtatgttggtgatgagatcaatccaaaattggcgcaaactgaagctggaattcttcatgagtcagtgaaagagaagaaaggaagccatgaagaagccataaggatcttgactacaaggagcaaaacccagctgattgcgacattcaaccgctacagagagactcatggtacttccatcactaagaagctgttggatgaaggatctgatgaatttcagaaggcattgtataccaccatccgtagcttcaatgatcatgttaagtactatgaaaaggtggtgcgagatgcaatcaagaaggttggaactgatgaggatgcgctgacccgtgtgattgtgagcagagctcagcatgacctaaaagtcatctcagatgtttactacaaaagaaacagtgttcttcttgagcatgttgtggccaaggaaacttcaggggattacaagaagtttcttctcactctcttggggaaagaggaatga

SEQ ID NO: 52 - protein, 313 aa, Medicago truncatula
MATLSAPSNHSPNEDAEALRKAFEGWGTDEKTVITILGHRNSNQIQQIRKAYEGIYNEDLIKRLESEIKGDFEKAVYRWILEPAERDAVLANVAIKSGKNYNVIVEISAVLSPEELLNVRRAYVKRYKHSLEEDLAAHTSGHLRQLLVGLVTAFRYVGDEINPKLAQTEAGILHESVKEKKGSHEEAIRILTTRSKTQLIATFNRYRETHGTSITKKLLDEGSDEFQKALYTTIRSFNDHVKYYEKVVRDAIKKVGTDEDALTRVIVSRAQHDLKVISDVYYKRNSVLLEHVVAKETSGDYKKFLLTLLGKEE

SEQ ID NO: 53 - DNA, 936 bp, Medicago truncatula
atggctaccctttctgctcctagcaaccattctcccaatgaagatgctgaagctctaagaaaggctttcgaaggatggggtactgatgagaagacagtgataacaatacttggtcatagaaactctaatcagatacagcaaatcagaaaagcttatgaaggcatttacaatgaggatcttatcaaacgattggaatctgagatcaaaggagactttgagaaagctgtgtaccgttggattcttgaacctgcggagcgtgatgctgttttggcaaatgttgctatcaagagtggcaaaaattacaatgtcattgtggaaatttctgctgttctctcccccgaagagctcttgaatgtgagacgtgcttacgtcaaacgctacaagcactccttggaagaagatttggctgcccatacttccggccatcttcgccaggcaaggctagtgactgcatttaggtatgttggtgatgagatcaatccaaaattggcgcaaactgaagctggaattcttcatgagtcagtgaaagagaagaaaggaagccatgaagaagccataaggatcttgactacaaggagcaaaacccagctgattgcgacattcaaccgctacagagagactcatggtacttccatcactaagaagctgttggatgaaggatctgatgaatttcagaaggcattgtataccaccatccgtagcttcaatgatcatgttaagtactatgaaaaggtggtgcgagatgcaatcaagaaggttggaactgatgaggatgcgctgacccgtgtgattgtgagcagagctcagcatgacctaaaagtcatctcagatgtttactacaaaagaaacagtgttcttcttgagcatgttgtggccaaggaaacttcaggggattacaagaagtttcttctcactctctgtcttaatattagctga

SEQ ID NO: 54 - protein, 311 aa, Medicago truncatula
MATLSAPSNHSPNEDAEALRKAFEGWGTDEKTVITILGHRNSNQIQQIRKAYEGIYNEDLIKRLESEIKGDFEKAVYRWILEPAERDAVLANVAIKSGKNYNVIVEISAVLSPEELLNVRRAYVKRYKHSLEEDLAAHTSGHLRQARLVTAFRYVGDEINPKLAQTEAGILHESVKEKKGSHEEAIRILTTRSKTQLIATFNRYRETHGTSITKKLLDEGSDEFQKALYTTIRSFNDHVKYYEKVVRDAIKKVGTDEDALTRVIVSRAQHDLKVISDVYYKRNSVLLEHVVAKETSGDYKKFLLTLCLNIS

SEQ ID NO: 55 - DNA, 939 bp, Glycine soja
atggctacccttattgctcccagcaaccactctccccaagaagatgctgaggctctcagaaaggcttttgaaggatgggggactgatgagaaaactgtcatagtaatattgggtcatagaactgtttatcagaggcagcaaatcagaagagtctatgaggaaattttccaggaggatcttgtgaaacgcctagagtctgagatcaaaggagactttgagaaagccgtgtaccgatggctactagaacctgcagatcgtgatgctgttttggccaatgttgccatcaagaatggcaaaaactacaatgtcattgtggaaatcgccactatcctctccccggaagagcttttggccgtgagacgtgcttatctcaaccgctacaagcactccttggaagaagatgttgcagctcatacctctggccatctacgccagcttttagtggggttggtgacctcatataggtatgttggtgatgagatcaatccaaaattggcacaaactgaggctgaaattcttcatgatgctgtgaaagagaagggtagctatgaagagaccattagggtcttgaccacaaggagcaagacccaacttgttgcaactttcaaccgctacagagagatacatggcacttccatctctaagaaactggtggatgaaggatctgatgagtttcaaagggcattgtacactgccattcgtgccatcaatgatcctattaagtactatgaaaaggtggtgcgcaatgcaatcaaaaaggttggaaccgatgaggatgcactcactcgcgtggttgtgagccgggccgagaaggacctgaagataatctcagaggtttactacaagagaaacagcgttcttcttgagcatgctattgccaaggaaatctcaggggactacaagaagttccttctcactctgttggggaaagaagactaa

SEQ ID NO: 56 - protein, 312 aa, Glycine soja
MATLIAPSNHSPQEDAEALRKAFEGWGTDEKTVIVILGHRTVYQRQQIRRVYEEIFQEDLVKRLESEIKGDFEKAVYRWLLEPADRDAVLANVAIKNGKNYNVIVEIATILSPEELLAVRRAYLNRYKHSLEEDVAAHTSGHLRQLLVGLVTSYRYVGDEINPKLAQTEAEILHDAVKEKGSYEETIRVLTTRSKTQLVATFNRYREIHGTSISKKLVDEGSDEFQRALYTAIRAINDPIKYYEKVVRNAIKKVGTDEDALTRVVVSRAEKDLKIISEVYYKRNSVLLEHAIAKEISGDYKKFLLTLLGKED

SEQ ID NO: 57 - DNA, 942 bp, Glycine max
atggctacccttattgctcccagcaaccactctccccaagaagatgctgaagctctcagaaaggcttttgaaggatggggaactgatgagaacactgtcatagtaattttgggtcatagaactgtgtatcagaggcagcaaatcagaagagtttatgaggaaatttaccaggaggatcttgtgaagcgcctagagtctgagatcaaaggagactttgagaaagccgtgtaccgatggatactggaacctgcagatcgtgatgctgttttggccaatgttgccatcaagagtggcaaaaactacaatgtcattgtggaaattgccactatcctctccccggaagagcttttggcggttagacgcgcctatctcaaccgctacaagcactccttggaagaagatgttgcagctcatacctctggccatctacgccagcttttagtggggttggtgaccgcattcaggcatgttggtgatgagatcaatccaaaattggcacaatctgaggctgaaattcttcatgatgctgtaaaagagaagaagggtagctatgaagagaccattagggtcttgactacaaggagcaggacccaacttgttgcaactttcaaccgctacagagagatccatggcacttccatctctaagaaattggtggatgaaggatctgatgagtttcagagggcattgtacactgccattcgtgccatcaatgatcctattaagtactatgaaaaggtggtgcgcaatgcaatcaaaaaggttggaaccgatgaggatgcactcactcgcgtggttgtgagccgggccgagaaggacctgaagataatctcagaggtttactacaagagaaacagcgttcttcttgagcatgctattgccaaggaaatctcaggggactacaagaagttccttctcactctgttggggaaagaagactaa

SEQ ID NO: 58 - protein, 313 aa, Glycine max
MATLIAPSNHSPQEDAEALRKAFEGWGTDENTVIVILGHRTVYQRQQIRRVYEEIYQEDLVKRLESEIKGDFEKAVYRWILEPADRDAVLANVAIKSGKNYNVIVEIATILSPEELLAVRRAYLNRYKHSLEEDVAAHTSGHLRQLLVGLVTAFRHVGDEINPKLAQSEAEILHDAVKEKKGSYEETIRVLTTRSRTQLVATFNRYREIHGTSISKKLVDEGSDEFQRALYTAIRAINDPIKYYEKVVRNAIKKVGTDEDALTRVVVSRAEKDLKIISEVYYKRNSVLLEHAIAKEISGDYKKFLLTLLGKED

SEQ ID NO: 59 - DNA, 942 bp, Phaseolus coccineus
atggctacccttattgcccccagcaaccactctccccaagaagatgctgaagctctcagaaagtcttttgaaggatggggtactgatgagaactctataatagcaattttgggtcatagaactgttcatcagaggcaacaaatcagaaaagcttatgaggaaatttaccaggaggatcttgtgaagcgcttggagtctgagatcaaaggagactttgagaaagctgtgtaccgatggatactggaacctgcagatcgtgatgctgttttggccaatgttgccatcaagagtggcaaaaactacaatgtcattgtggaaattgccactataaactccccagaagagcttttggctgtcagacgtgcttatctccaccgctacaagcactctttggaagaagatcttgcagcacataccactggcccactacgccagcttctagtggggttggtgacctcattcaggtatgttggtgatgagataaatccaaaattggcacaaaatgaggctgaaattcttcatgatgctgtgaaagagaagaagagttcctatgaagaggccatcagggtcttgactacaaggagcaagactcaactggttgcaactttcaaccgttacagagagatccatggaggttccatctctaagaaactggtggatgaaggatctgatgattttgagaaggcattgcacaccaccatccgttccatcaatgatcatttcaagtactatgaaaaggtggtgagaaatgctatcaagaaggttggaactgatgaagatgcactcacccgtgtggttgtgagcagggctgagaaggatctgaaaacagtctcagaggtttactacaagagaaacagtgttcttcttgagcatgccatagccaaggaaatttcaggggattacaagaagttccttctcactctgttggggaaagaggactaa

SEQ ID NO: 60 - protein, 313 aa, Phaseolus coccineus
MATLIAPSNHSPQEDAEALRKSFEGWGTDENSIIAILGHRTVHQRQQIRKAYEEIYQEDLVKRLESEIKGDFEKAVYRWILEPADRDAVLANVAIKSGKNYNVIVEIATINSPEELLAVRRAYLHRYKHSLEEDLAAHTTGPLRQLLVGLVTSFRYVGDEINPKLAQNEAEILHDAVKEKKSSYEEAIRVLTTRSKTQLVATFNRYREIHGGSISKKLVDEGSDDFEKALHTTIRSINDHFKYYEKVVRNAIKKVGTDEDALTRVVVSRAEKDLKTVSEVYYKRNSVLLEHAIAKEISGDYKKFLLTLLGKED

SEQ ID NO: 61 - DNA, 942 bp, Glycine max
atggctacccttattgctcccagcaaccactctccccaagaagacgctgaggctctcagaaaggcttttgaaggatgggggactgatgagaaaactgtcatagtaatattgggtcatagaactgtttatcagaggcagcaaatcagaagagtctatgaggaaattttccaggaggatcttgtgaaacgcctagagtctgagatcaaaggagactttgagaaagccgtgtaccgatggatactagaacctgcagatcgtgatgctgttttggccaatgttgccatcaagaatggcaaaaactacaatgtcattgtggaaattgccactatcctctccccggaagagcttttggcggttagacgcgcctatctcaaccgctacaagcactccttggaagaagatgttgcagctcatacctctggccatctacgccagcttttagtggggttggtgaccgcattcaggcatgttggtgatgagatcaatccaaaattggcacaatctgaggctgaaattcttcatgatgctgtaaaagagaagaagggtagctatgaagagaccattagggtcttgactacaaggagcaggacccaacttgttgcaactttcaaccgctacagagagatccatggcacttccatctctaagaaattggtggatgaaggatctgatgagtttcaaagggcattgtacactgccattcgtggcatcaaggatcctattaagtactacgaaaaggtggtgcggaatgcaatcaaaaaggttggaactgatgaggatgcactcacccgtgtggtcgtgagccgggctgagaaggacttgaagataatctcagaggtttactacaagagaaacagtgttcttcttgagcatgctattgccaaggaaacctctggggactacaagaagttccttctcacactgttgggaaaagaagactaa

SEQ ID NO: 62 - protein, 313 aa, Glycine max
MATLIAPSNHSPQEDAEALRKAFEGWGTDEKTVIVILGHRTVYQRQQIRRVYEEIFQEDLVKRLESEIKGDFEKAVYRWILEPADRDAVLANVAIKNGKNYNVIVEIATILSPEELLAVRRAYLNRYKHSLEEDVAAHTSGHLRQLLVGLVTAFRHVGDEINPKLAQSEAEILHDAVKEKKGSYEETIRVLTTRSRTQLVATFNRYREIHGTSISKKLVDEGSDEFQRALYTAIRGIKDPIKYYEKVVRNAIKKVGTDEDALTRVVVSRAEKDLKIISEVYYKRNSVLLEHAIAKETSGDYKKFLLTLLGKED

SEQ ID NO: 63 - DNA, 945 bp, Unknown
  Synthetic sequence
atggctacccttgttgctccaaaccaaaaatctccggttgaagacgttgaggctctacataaggctttcaaaggatgggggactgatgagaagaccgttatagcaatattgggtcacagaaatgttcatcagaggcaacaaatcagaaagatttatgaggaaatttaccaagaggacctcatcaaacgcctagagtctgagctctctggtgactttgagagagctgtgtaccgttggatgctggaacctgcagatcgtgatgctgttttggccaatgttgccatcaagaatggcagcaaaggttaccatgtgattgtggaaattgcttgtgtgctttcagctgaagaggtcttggcagtgaagcgtgcctatcacaaccgttacaagagatctttggaagaagatgtagctactaataccactggtgacattcgccagctgttggttgggttggtgacagcatataggtatgatggagatgagatcaatgcaaaattggccaaaactgaagctgatattcttcatgaatctatcaaggagaaaaagggcaaccatgaagaagccatcaggatcctcaccacaaggagcaagacacaacttctggcaactttcaaccgctacagagatgaccatggcgcttccataactaagaaattgttggataatgcatctactgacttccagaaagcattgcacactgctattagatgtatcaatgaccacaaaaagtactatgaaaaggttctgcgcaatgcgataaaaggggttggaactgatgaggatgcactcacccgtgtggtggtctcaagggctgagaaggacctcagggacatcaaagagctttattataagagaaatagtgttcaccttgaggatgcagtggccaaggaaatctcaggggactacaagaagttccttctcactctgttggggaaagaagactaa

SEQ ID NO: 64 - protein, 314 aa, Unknown
  Synthetic sequence
MATLVAPNQKSPVEDVEALHKAFKGWGTDEKTVIAILGHRNVHQRQQIRKIYEEIYQEDLIKRLESELSGDFERAVYRWMLEPADRDAVLANVAIKNGSKGYHVIVEIACVLSAEEVLAVKRAYHNRYKRSLEEDVATNTTGDIRQLLVGLVTAYRYDGDEINAKLAKTEADILHESIKEKKGNHEEAIRILTTRSKTQLLATFNRYRDDHGASITKKLLDNASTDFQKALHTAIRCINDHKKYYEKVLRNAIKGVGTDEDALTRVVVSRAEKDLRDIKELYYKRNSVHLEDAVAKEISGDYKKFLLTLLGKED

SEQ ID NO: 65 - DNA, 945 bp, Glycine max
atggctacccttgttgctccaaaccaaaaatctccggttgaagacgcagaagctcttagaaaagctttccaaggatgggggaccgatgagaaaaccgttattgcaatactgggtcatagaaatgttcatcagaggcaacaaatcagaaaggtttatgaggaaatttaccaagaggacctcattaaacgcctggagtctgagctctctggtgactttgagagagctgtgtaccgttggatgctggaacctgcagatcgtgatgctgttttggccaatgttgccatcaagaatggcagcaaaggttaccatgtgattgtggaaattgcttgtgtgctttcagctgacgaggtcttggcagtgaagcgtgcctatcacaaccgttacaagagatctttggaagaagatgtggctactaataccactggtgatattcgccagctgttggttgggttggtgacagcatataggtatgatggtgatgaggtcaatgcaaaattggccaaaactgaagctgatattcttcatgaatctatcaaggagaaaaagggcaaccatgaagaagccatcaggatcctcaccacaaggagcaagacacaacttctggcaactttcaaccgctacagagatgaccatggcgcttctataactaagaaattgttggataatgcatctactgactttcagaaggcattgcacactgctattagatgtatcaatgaccacaaaaagtactatgaaaaggttctgcgcaatgcgataaaagaggttggaactgatgaggatgcactcacccgtgtggtggtctcaagggctgagaaggacctcagggacatcaaagagctttattataagagaaatagtgttcaccttgaggatgcagtggccaaggaaatctcaggggactacaagaaattcatcctcactctgttggggaaggaagattga

SEQ ID NO: 66 - protein, 314 aa, Glycine max
MATLVAPNQKSPVEDAEALRKAFQGWGTDEKTVIAILGHRNVHQRQQIRKVYEEIYQEDLIKRLESELSGDFERAVYRWMLEPADRDAVLANVAIKNGSKGYHVIVEIACVLSADEVLAVKRAYHNRYKRSLEEDVATNTTGDIRQLLVGLVTAYRYDGDEVNAKLAKTEADILHESIKEKKGNHEEAIRILTTRSKTQLLATFNRYRDDHGASITKKLLDNASTDFQKALHTAIRCINDHKKYYEKVLRNAIKEVGTDEDALTRVVVSRAEKDLRDIKELYYKRNSVHLEDAVAKEISGDYKKFILTLLGKED

SEQ ID NO: 67 - DNA, 942 bp, Medicago truncatula
atggctactcttgttacaataaaatcatctcctgtggaagatgctgaggctctccaaaaggcctttaaaggatgggggactgatgagaagagtgttatcacaatattaggtcatagaaatgtttatcagagacaacaaatcagaaaatcttatcaggaaatttaccaagaggatatcctcaaacgccttgaatctgaactctctggtgactttgagagagctgtgtaccgttggatgctggaacctgcggatcgcgatgctgttttggccaatgtagccatcaaggatggaagcaaaagttaccatgtgattattgaaattgtttctgttctttcacctgaagaagtgttggcaatgagacgtgcttatcataaccgttacaagcattctttagaagaagatttagctgctcataccaccggtcatcttcgccagctgttggttggattggtgacctcgtttagatatggcggtgctgagataaatccgaaattggcaaaaactgaagctgacattcttcatgaatcaatcaaagagaaaaaaggaaatcatgaagaagccattagaatccttactacaaggagcaagactcagcttttggcaactttcaaccgctacagagatgaccatggcatttccataactaagaaactgttggataatgcatctgatgattttcacaaggcattgcacactacgattcgatgcatcaatgaccataagaagtactatgaaaagattctacgcggtgcgttgaaaagggttggaaccgatgaggatggactgactcgtgtggtcgtgacaagagccgagaaagatctgaaggacatcaaagagctgtattataagagaaacagtgttcaccttgaggatgcagtggcaaaggaaatttcaggagactacaagaagttcatccttaccttgttggggaagcaagattga

SEQ ID NO: 68 - protein, 313 aa, Medicago truncatula
MATLVTIKSSPVEDAEALQKAFKGWGTDEKSVITILGHRNVYQRQQIRKSYQEIYQEDILKRLESELSGDFERAVYRWMLEPADRDAVLANVAIKDGSKSYHVIIEIVSVLSPEEVLAMRRAYHNRYKHSLEEDLAAHTTGHLRQLLVGLVTSFRYGGAEINPKLAKTEADILHESIKEKKGNHEEAIRILTTRSKTQLLATFNRYRDDHGISITKKLLDNASDDFHKALHTTIRCINDHKKYYEKILRGALKRVGTDEDGLTRVVVTRAEKDLKDIKELYYKRNSVHLEDAVAKEISGDYKKFILTLLGKQD

SEQ ID NO: 69 - DNA, 945 bp, Gossypium hirsutum
atggccaccattgatgttcctgagcaggtttcagttcttgcagatgcagaagctcttagaaaggcttgtaaaggatgggggacagatgagaaggcaataatttcagtgttgggtcacagaaatgcagttcaaaggaagcaaatcaggcttgcttatgaagatctttaccaggaagatctcatcaagcgtcttgaatcagagctttctggtgatttcgagaaagcagtgtatcgttggatattggatccagcagatcgagatgctgtgttagctaatgttgccataaagaaattaagccctgatcaccatgtgatcgttgaaatttcatgcaccagatcccctgaagagctcttggctgtgaggagagcttatcaggctcgatacaagcattccctggaagaagatgtggctgctcataccaagggagacactcgaaagctcttggtttccttagtgagcgcatttagatacgatggggaagaaataaataccagggtagcaaattctgaagccaaaattctccatgaagctgtcaaagacaaggaattcaaccatgaagaaattatcaggatcttgagtacaagaagcaagatgcaactcatggcgactttcaatcgttacagagatgatcatggcactaccattaccaagaacttggaaggtgattcaggtgacgaattcctcaagacactgcgtccgacgattcgatgccttaatgacccgaaaaagtatttcgaaaaggttttgcgcaattcgatccggagggtcggaaccgatgaggatgcacttaccagagtaattgttacaagagctgaaaaggacctgaaagatgtcaaagagctttattacaagagaaacagtgtgcctcttgatcaagctgttgctaaggacaccacaggggattacaaggcattgcttcttactctgttgggaaaagaagattga

SEQ ID NO: 70 - protein, 314 aa, Gossypium hirsutum
MATIDVPEQVSVLADAEALRKACKGWGTDEKAIISVLGHRNAVQRKQIRLAYEDLYQEDLIKRLESELSGDFEKAVYRWILDPADRDAVLANVAIKKLSPDHHVIVEISCTRSPEELLAVRRAYQARYKHSLEEDVAAHTKGDTRKLLVSLVSAFRYDGEEINTRVANSEAKILHEAVKDKEFNHEEIIRILSTRSKMQLMATFNRYRDDHGTTITKNLEGDSGDEFLKTLRPTIRCLNDPKKYFEKVLRNSIRRVGTDEDALTRVIVTRAEKDLKDVKELYYKRNSVPLDQAVAKDTTGDYKALLLTLLGKED

SEQ ID NO: 71 - DNA, 939 bp, Glycine max
atggccacccttgttgttcataaccaaacttcttctgctcaagatgctgaggctctccaacaggctttcaaaggatggggggctgatgacaaggccattatagcaatactgggtcacagaaatgttcatcagaggcaagaaatcagaaaagcttatgaggagatttaccaagaggatctcatcaaacgccttgagtccgagatctctggtgactttgagagagctatgtaccggtggatgttgcagcctgcggatcgtgatgctgttttggtgaatgtggcaatcaagaatggcaccaaagactaccatgtgattgcagagattgcttgtgtcctatcagctgaagagctcttggcagtgaggcgggcctaccaccgccgctacaagtgttccttggaggaagatgtggcagctaacaccaccggcaatcttcgccagcttttggttggattggtgacctcatataggtatgagggtgatgagatcaatgtaaaattttcacaaactgaagctaatgttcttcatgaatctgtcaaggagaagaaaggcaacagtgaagaagtcatcaggatccttactacaagaagcaaaactcaacttgtggcaactttcaaccgctacagagatgaacatggaatttccatcagtaagaaattgttggatcaaacatctgatgacttccacaaggtgttgcacactgctattcgctgcatcaatgaccataagaagtactatgagaaggttttgcgcaatgcagtcaagaagtttggaactgacgaggatggactaagccgtgtgattgttacaagggctgagaaggatctgaaggacatcaaagagctctactacaagagaaacagtgttcaccttgaggatgaagtgtccaaggaaacttctggggactacaagaagttcctcctcactctcttggggaaataa

SEQ ID NO: 72 - protein, 312 aa, Glycine max
MATLVVHNQTSSAQDAEALQQAFKGWGADDKAIIAILGHRNVHQRQEIRKAYEEIYQEDLIKRLESEISGDFERAMYRWMLQPADRDAVLVNVAIKNGTKDYHVIAEIACVLSAEELLAVRRAYHRRYKCSLEEDVAANTTGNLRQLLVGLVTSYRYEGDEINVKFSQTEANVLHESVKEKKGNSEEVIRILTTRSKTQLVATFNRYRDEHGISISKKLLDQTSDDFHKVLHTAIRCINDHKKYYEKVLRNAVKKFGTDEDGLSRVIVTRAEKDLKDIKELYYKRNSVHLEDEVSKETSGDYKKFLLTLLGK

SEQ ID NO: 73 - DNA, 945 bp, Phaseolus coccineus
atggccactcttgttcttcatcatcaaacttctcctgctgaagatgctgaagttcttcaccaagctttcaaaggatggggttctgatggcaaggccattatagcaatactgggtcacagaaatgttcatcagagacaggagatcagaaaggcttatgaagagatttaccaagagaatctcatcaaacgacttgagtctgagatctctggtgattttgagagagctgtgtatcggtggatgttggaggctgcagatcgtgatgctgttttgattaatgtagcaatcaagaatggtcctaaagagtaccatgtgattgcagagatttccagtgttctttcagccaaagaactcttggcagtgagatctgcttatcatgatcgctacaaacgttccatggaagaagatgtagcagctcacaccacaggccatcttcgtcagcttttggttgggttggtgagttcgtataggtatgagggtgatgacatccatgcaaaattggcacaaactgaagctaatattcttcatgaatcaattaaggagaagaaagccaaccatgaagaagttatcagaattcttaccaccagaagcaaaactcaacttgtagcaaccttcaaccgctacagagatgagcatggaatttccatcactaagaaattggtggatcagtcatctgatgattttgtcaaggtattgcacacagtgattcgctgcatcaatgaccccaggaaatactatgagaaggttctgcgcaatgcaatgaaaaaatttggaactgatgaggatgggttgactcgtgtgattgtgacaagggctgagaaagatttgaaggatataaaggagatctactacaaaaggaacagtgttcatcttgaagatgatgtctccaaggaaatctcaggagactacaagaggttcatcctcactctcctggggaaccaagattag

SEQ ID NO: 74 - protein, 314 aa, Phaseolus coccineus
MATLVLHHQTSPAEDAEVLHQAFKGWGSDGKAIIAILGHRNVHQRQEIRKAYEEIYQENLIKRLESEISGDFERAVYRWMLEAADRDAVLINVAIKNGPKEYHVIAEISSVLSAKELLAVRSAYHDRYKRSMEEDVAAHTTGHLRQLLVGLVSSYRYEGDDIHAKLAQTEANILHESIKEKKANHEEVIRILTTRSKTQLVATFNRYRDEHGISITKKLVDQSSDDFVKVLHTVIRCINDPRKYYEKVLRNAMKKFGTDEDGLTRVIVTRAEKDLKDIKEIYYKRNSVHLEDDVSKEISGDYKRFILTLLGNQD

SEQ ID NO: 75 - DNA, 945 bp, Phaseolus vulgaris
atggccactcttgttcttcatcatcaaacttctcctgctgaagatgctgaagttcttcatcaagctttcaaaggatggggttctgatggcaaggccattatagcaatactgggtcacagaaatgttcatcagagacaggagatcagaaaggcttatgaagagatttaccaagagaatctcatcaaacgtcttgagtctgagatctctggtgattttgagagagctgtgtatcggtggatgttggaggctgcagatcgtgatgctgttttgattaatgtagcaatcaagaacggtcctaaagagtaccatgtgattgcagagatttccagtgttctttcagccaaagaactcttggcagtgaggtctgcttatcatgatcgctacaaacgttccatggaagaagatgtagcagctcacaccactggccatcttcgtcagcttttggtggggttggtgagttcatacaggtatgagggtgatgacatccatgcaaagttggcacaaactgaagctaatattcttcatgaatcaattaaggagaagaaagccaaccatgaagaagttatcagaattcttaccaccagaagcaaaactcaacttgtagcaaccttcaaccgctacagagatgagcatggaatttccatcactaagaaattggtggatcagtcatctgatgattttgtcaaggttttgcacacagtgattcgctgcatcaatgaccccaggaaatactatgagaaggttttgcgcaatgcaatgaaaaaatttggaactgatgaggatgggttgactcgtgtgattgtgacaagggctgagaaagatttgaaggatataaaggagatctactacaaaaggaacagtgttcatcttgaagatgatgtctccaaggaaatctcaggagactacaagaggttcatcctcactctcctggggaaccaagattag

SEQ ID NO: 76 - protein, 314 aa, Phaseolus vulgaris; identical to SEQ ID NO: 74

SEQ ID NO: 77 - DNA, 945 bp, Medicago truncatula
atggccaccattgttgttcatagccaaacatctccagttcaagatgctgaggctcttcgacttgctttcaaaggatggggggctgataacaaggccattattgcaattttgggtcatagaaatgttcatcagaggcaacagattagaaaagcttatgaggagcttttcgaagaggatctcatcaaacgtcttgagtcagaaatctccggtgactttgagagggccgtgtaccggtggatgttggaccctgcagaccgtgacgctgttttgatcaatgtagcaatcaggaatggcaataaagactatcatgtggttgctgaaattgcttctgtgctatcaactgaagagctcttggcagtgaggcgtgcctatcataaccgctacaagcgttccatcgaagaagacgtgtctgctcacactaccggtcatcttcgccagcttttggttggattggtgagctcatttaggtatgagggagatgagatcaatgcaaaattggcacaaactgaggctaatattattcatgaaagtgtcaaggaaaagaaaggcaacaatgaagaagtcattaggattcttaccacaaggagcaagactcaacttgtggcaactttcaaccgttatagggatgaacatggcatctccattagtaagaaattgcttgatcaaacatctgatgacttccagaagacattgcacacagctattcgttgcatcaatgaccacaaaaagtactatgagaaggttttgcgcaatgcgatcaagaaatttggaaccgatgaagatggactgagccgcgtgattgtcacaagggctgaaaaggatttgagggacatcaaagagctgtactacaagagaaacagtgttcatcttgaagatgaagtttccaaggaaacctcaggggactacaagaagttcatcctcactcttttggggaaacatgattaa

SEQ ID NO: 78 - protein, 314 aa, Medicago truncatula
MATIVVHSQTSPVQDAEALRLAFKGWGADNKAIIAILGHRNVHQRQQIRKAYEELFEEDLIKRLESEISGDFERAVYRWMLDPADRDAVLINVAIRNGNKDYHVVAEIASVLSTEELLAVRRAYHNRYKRSIEEDVSAHTTGHLRQLLVGLVSSFRYEGDEINAKLAQTEANIIHESVKEKKGNNEEVIRILTTRSKTQLVATFNRYRDEHGISISKKLLDQTSDDFQKTLHTAIRCINDHKKYYEKVLRNAIKKFGTDEDGLSRVIVTRAEKDLRDIKELYYKRNSVHLEDEVSKETSGDYKKFILTLLGKHD

SEQ ID NO: 79 - DNA, 1674 bp, Glycine max
atggccatgatgtggattagagtcatacttatactagcggcttgcatgcttcccctttcagtggaagccatggttcgccactacaagttcaacgtggtggtgaagaatgtcacaagattgtgttcaacaaagcccatagtaaccgtaaatggaaagttcccgggccccaccatctatgctagggaagatgacactgttttggtgaaggtggttaaccatgtcaaatacaatgttagcatccactggcatggagtgagacaattgaggacgggttgggccgatgggcctgcttacataacccaatgcccaattcaaccgggccaggcctttgtgtacaacttcaccctcacaggacagagagggacactttggtggcatgcacatatcctttggcttaggtcaactgtgcatggtgccttggtcattttgcctaagcttggagttccttacccttttcccaaaccccacactgaaaaagtcatcatattgagtgaatggtggaaatctgatactgaggctgtaataaatgaagctttgaaatctgggttggctccaaatgtctctgatgctcacacaatcaatggtcatccaggatccgttcaaaattgtgcatcacaagggggatacgaacttcaagttcaacctggaaacacctacttactaagaataatcaacgctgcactgaatgaagagttgttctttaaaattgctggccaccaactcacagttgttgaggttgatgctgtctacacaaaacctttcaaaactgacaccatagttatagcaccaggccaaaccacaagtgtgcttctaaaagccaaccgtgccgctggcaaatacttagtagcagccactcctttcatggattctcctattgcagtggacaatgtgactgccactgccacattgcactacactggctcactaggttccaccatcacaaccctcacttcattgcctcccaaaaatgccacaccagttgctacaaacttcactgactcactcagaagcctaaactccaaaaagtaccctgctagagtgcctcaaaaggttgaccattccttgttcttcactatcagccttggagtcaacccttgccccacttgtgtcaatggtagcaaagtggttgcagctatcaacaatgtgacctttgtgatgcctaaagtttctctcctccaagcacatttcttcaacataagtggagttttcattgatgattttcctgggaagcctccagtggtttatgacttcacagggacacaacagcctacaaatttgaggactaatagagggacaagggtttatagacttgcctataactccactgttcaattagtcttgcaagatactgggatgataacacctgagaatcatccccttcaccttcatggattcaacttctttgtggttggtaggggacaagggaacttcaaccccaaaaaggacaccaaaaagtttaatcttgtggatcctgtggagagaaacacagttggtgttccatctgggggatggactgctatcagattcagagctgataatccaggtgtttggtttatgcattgccatttggaaattcatacaacatggggattgaagatggcatttgtggtggacaatggtaaaggaccaaatgagtctctactaccacctccaagtgatcttcccaagtgttga

SEQ ID NO: 80 - protein, 557 aa, Glycine max
MAMMWIRVILILAACMLPLSVEAMVRHYKFNVVVKNVTRLCSTKPIVTVNGKFPGPTIYAREDDTVLVKVVNHVKYNVSIHWHGVRQLRTGWADGPAYITQCPIQPGQAFVYNFTLTGQRGTLWWHAHILWLRSTVHGALVILPKLGVPYPFPKPHTEKVIILSEWWKSDTEAVINEALKSGLAPNVSDAHTINGHPGSVQNCASQGGYELQVQPGNTYLLRIINAALNEELFFKIAGHQLTVVEVDAVYTKPFKTDTIVIAPGQTTSVLLKANRAAGKYLVAATPFMDSPIAVDNVTATATLHYTGSLGSTITTLTSLPPKNATPVATNFTDSLRSLNSKKYPARVPQKVDHSLFFTISLGVNPCPTCVNGSKVVAAINNVTFVMPKVSLLQAHFFNISGVFIDDFPGKPPVVYDFTGTQQPTNLRTNRGTRVYRLAYNSTVQLVLQDTGMITPENHPLHLHGFNFFVVGRGQGNFNPKKDTKKFNLVDPVERNTVGVPSGGWTAIRFRADNPGVWFMHCHLEIHTTWGLKMAFVVDNGKGPNESLLPPPSDLPKC

SEQ ID NO: 81 - DNA, 1674 bp, Glycine max
atggccatgatgtggataagaatcatacttctagtagcggcttgcatgcttcctctttcagtggaagccatggttcgccactacaagttcaacgtggtggtaaagaatgtcacaagattgtgttcaacaaagcccatagtaaccgtaaatgggaagttccctggccccacaatatatgctagggaagatgacactgttttggtaaaggtggttaaccatgttaaatacaatgttagcatccactggcatggggtgagacaattgcagacgggttgggccgatgggcctgcatacataactcaatgcccaattcaaccgggccaggcctttgtgtacaacttcaccctcacagggcagagagggacactttggtggcatgcacatatcctttggcttaggtcaactgttcatggtgccttggtcattttgcctaagcttggagttccttacccttttcccaaaccccacacggaaaaagtcatcatattgagtgaatggtggaaatcagatactgaagctgtaataaatgaagctttgaaatctgggtcggctccaaatgtctctgatgctcacacaatcaatggtcatcctggatctgtacaaaattgtgcatcacaagggggatacaaactccaagttcaacctggaaacacctacttgctaagaataatcaacgctgcactgaatgaagagttgttctttaaaattgctggtcaccaactgacagttgttgaggttgatgctgtctacacaaaaccttttaaaactgacactatagttatagcacctggccaaaccacaaatgtgcttctaaaagcaaaccgtgctgctggcaaatacttagtagcagccactcccttcatggattctcctattacagtggacaatgtgactgccactgccacattacactacacaggctcacttggttccaccatcacaaccctcacttcattgcctcccaaaaatgccacaccagttgctacaaacttcactgactcactcagaagcctaaactccaaaaagtaccctgctagagtgcctcaaaaggttgaccattccttgttcttcactgtcagccttggagtcaacccttgccccacatgtgccaatggtagtaaagtggttgcagccatcaacaatgtgacctttgttatgcctaaagtttctctcctccaagcacatttcttcaacataagtggagttttcacagatgattttcctgggaagcctccagtggtttatgacttcacagggacacaacagccaacaaatttgaggactaatagagggacaagggtatatagacttgcctataactccacagttcaattagtcttgcaagatactggaatgataacacctgagaaccatcctattcatctccatggattcaacttcttcgtggttggtaggggacaagggaatttcaaccccaaaaaggacaccaaaaagtttaatcttgtggatcctgtggagagaaacacagttggtgttccatctggggggtggactgctatcagattcagggctgataatccaggtgtttggtttatgcattgccatttagaaattcatacaacatggggattgaagatggcatttgtggtagacaatggtaaaggaccaaatgagtctctactaccacctccaagtgatcttcccaagtgttga

SEQ ID NO: 82 - protein, 557 aa, Glycine max
MAMMWIRIILLVAACMLPLSVEAMVRHYKFNVVVKNVTRLCSTKPIVTVNGKFPGPTIYAREDDTVLVKVVNHVKYNVSIHWHGVRQLQTGWADGPAYITQCPIQPGQAFVYNFTLTGQRGTLWWHAHILWLRSTVHGALVILPKLGVPYPFPKPHTEKVIILSEWWKSDTEAVINEALKSGSAPNVSDAHTINGHPGSVQNCASQGGYKLQVQPGNTYLLRIINAALNEELFFKIAGHQLTVVEVDAVYTKPFKTDTIVIAPGQTTNVLLKANRAAGKYLVAATPFMDSPITVDNVTATATLHYTGSLGSTITTLTSLPPKNATPVATNFTDSLRSLNSKKYPARVPQKVDHSLFFTVSLGVNPCPTCANGSKVVAAINNVTFVMPKVSLLQAHFFNISGVFTDDFPGKPPVVYDFTGTQQPTNLRTNRGTRVYRLAYNSTVQLVLQDTGMITPENHPIHLHGFNFFVVGRGQGNFNPKKDTKKFNLVDPVERNTVGVPSGGWTAIRFRADNPGVWFMHCHLEIHTTWGLKMAFVVDNGKGPNESLLPPPSDLPKC

SEQ ID NO: 83 - DNA, 1671 bp, Glycine max
atggcagcgtttgggattcgaatcatgctattgatggcagcttgcttgcttccactatctgtggaagctatggttcgccactacaagttcaatgtggtgctgaagaatgccacaagattgtgttcaacaaagccaattgtcaccgtaaatggaaagttcccaggtcccaccatctatgctagggaagatgacactgtactggttaaggtggtcaatcatgtcaagtacaatgttagcatccactggcatggggtcagacaattgagaacaggttgggctgatgggccagcatacataacccagtgtccaattcaaccgggccaggcttttatctacaactttacccttacaggccaaagaggcacactttggtggcatgcacatatcctctggcttagggccactgtccatggtgccttggtcatcttgcctaagcttggagttccttaccctttccccaaaccaaatatggaacaagttatgatattgagtgaatggtggaaatcagatactgaggctgtaataaatgaagctttgaaatctggtttggcaccaaatgtctccaatgctcacacaatcaatggccacccaggacctgtccaaggttgtgcttcacaagaaggatttaagttggatgtccaaccaggaaacacctacttgctaagaatcatcaatgctgcactcaatgaagagctattcttcaaaattgctggccatgaactcactgttgttgaggttgatgcagtctacacaaaaccattcaaaactgataccattgtcatagcacctggccagaccacaaatgtgcttctaacaaccaaacatgcagcaggcaaatacttggttgcagcctctccttttatggatgctcccattgcagttgacaacaagactgccactgccacattacactactcaggcacccttggttccaccatcaccaccctcacttccatgcctcccaaaaatgctacccctcttgccaccagtttcactgactcactcagaagcttgaactccaaaaagtatcctgctagagtgcctttgaagattgaccacaacttgctcttcactgttagtcttggaattaacccttgtgctacttgtgtgaataacagcagggtggtggcagatatcaacaatgttacctttgtgatgcctaaaatttctcttctccaagcacatttcttcaaaattaagggagttttcactgatgattttcctggaaatcctcctgtggtttataacttcacagggacacagccatcaaatttgaggaccatgaaaggaacaagggtgtatagacttgcttacaattctacagttcaattagtcttgcaagatactggaatgataacacctgagaaccaccccattcatctccatggctttaatttctttgtagttggtaggggacagagaaatttcaaccccacaaaagaccccaagaaatttaaccttgtagatcctgtggagagaaatacagttggagtcccagctggagggtggactgctatcagattcaggactgataatccaggtgtttggtttatgcattgtcatttggaaattcatacaacatggggactgaagatggcttttgttgtggataatggtaaaggaccaaatgaatctctattaccacctccaagtgacctccccaagtgttga

SEQ ID NO: 84 - protein, 556 aa, Glycine max
MAAFGIRIMLLMAACLLPLSVEAMVRHYKFNVVLKNATRLCSTKPIVTVNGKFPGPTIYAREDDTVLVKVVNHVKYNVSIHWHGVRQLRTGWADGPAYITQCPIQPGQAFIYNFTLTGQRGTLWWHAHILWLRATVHGALVILPKLGVPYPFPKPNMEQVMILSEWWKSDTEAVINEALKSGLAPNVSNAHTINGHPGPVQGCASQEGFKLDVQPGNTYLLRIINAALNEELFFKIAGHELTVVEVDAVYTKPFKTDTIVIAPGQTTNVLLTTKHAAGKYLVAASPFMDAPIAVDNKTATATLHYSGTLGSTITTLTSMPPKNATPLATSFTDSLRSLNSKKYPARVPLKIDHNLLFTVSLGINPCATCVNNSRVVADINNVTFVMPKISLLQAHFFKIKGVFTDDFPGNPPVVYNFTGTQPSNLRTMKGTRVYRLAYNSTVQLVLQDTGMITPENHPIHLHGFNFFVVGRGQRNFNPTKDPKKFNLVDPVERNTVGVPAGGWTAIRFRTDNPGVWFMHCHLEIHTTWGLKMAFVVDNGKGPNESLLPPPSDLPKC

SEQ ID NO: 85 - DNA, 1671 bp, Glycine max
atggcagcgtttggtattcaaatcatgctattgctggcagctttcttgcttccactatctgtggaagctatggttcgccactacaagttcaatgtggtgcaaaagaataccacaagattgggttcaaccaagcccattgttaccataaatggaaagttcccaggtcccaccatctatgcaagggaagatgacactgttctggttaaggtagtcaaccaggtcaagtacaatgtcagcatccattggcatggggtgagacaattgagaacaggttgggctgatgggccagcatacataacccagtgtccaattctaccgagccaggcctatgtctacaactttactcttacaggccagagaggcacactttggtggcatgcacatatcctctggcttagggccactgtccatggtgccttggtcatcttgcccaagcttggagttccttacccttttcccaaaccaaatatggaacaagttatcatattgagtgaatggtggaaatcagatactgaggctgtaataaatgaagctttgaaatctggtttggctccaaatgcctctgatgctcacacaatcaacggccatccaggacctatccaaggctatgcttcacaaggaggatataagttggatgttcaaccaggaaagacctacttgctaagaatcatcaatgctgcactcaatgaagagctcttctttaaaattgctgggcatgaactcactgttgttgaggttgatgcagtttacacaaaacctttgaaaactgataccattgtcatagcacctggccaaaccacaaatgtgcttctaacaaccaaacatgcaactggcaaatacttggttgcagcctctcctttcatggatgctcctattgcagttgacaacaagactgccactgccactttacactatctaggcacccttggttccaccatcaccaccctcacttccatgcctcctaaaaatgcaacaccagttgccaccactttcatcgactctctccgaagcttaaactccaaagagcatcctgctagagtccctttaaagattgatcataacttgctcttcacagttagccttggtgtcaacccttgtgctacttgtgtgaataatagcagggtggtagcagatatcaacaatgttacctttgtgatgcctaaaatttctcttcttcaagcacatttcttcaagatcaagggagttttcaccgacgatttccccggaaatcctcctgtggtgtataacttcacagggacacaaccatcaaatttgaagaccatgaaaggcacaagggtctatagacttgcttacaattccacagttcaattggtcttgcaagatactggaatgataacacctgagaaccatcctattcatctccatggcttcaacttttttgtggttggtaggggacaagggaatttcaaccccacaaaagaccccaagaaatttaaccttgtagatcctgtggagagaaatacagttggagtcccggctggggggtggactgctattagattcagggctgacaatccaggtgtctggtttatgcattgccacttggaaattcatacaacatggggactgaagatggcttttgttgtggacaacggtaaaggaccaaatgaatctttattaccacctccaactgacctacccaagtgttga

SEQ ID NO: 86 - protein, 556 aa, Glycine max
MAAFGIQIMLLLAAFLLPLSVEAMVRHYKFNVVQKNTTRLGSTKPIVTINGKFPGPTIYAREDDTVLVKVVNQVKYNVSIHWHGVRQLRTGWADGPAYITQCPILPSQAYVYNFTLTGQRGTLWWHAHILWLRATVHGALVILPKLGVPYPFPKPNMEQVIILSEWWKSDTEAVINEALKSGLAPNASDAHTINGHPGPIQGYASQGGYKLDVQPGKTYLLRIINAALNEELFFKIAGHELTVVEVDAVYTKPLKTDTIVIAPGQTTNVLLTTKHATGKYLVAASPFMDAPIAVDNKTATATLHYLGTLGSTITTLTSMPPKNATPVATTFIDSLRSLNSKEHPARVPLKIDHNLLFTVSLGVNPCATCVNNSRVVADINNVTFVMPKISLLQAHFFKIKGVFTDDFPGNPPVVYNFTGTQPSNLKTMKGTRVYRLAYNSTVQLVLQDTGMITPENHPIHLHGFNFFVVGRGQGNFNPTKDPKKFNLVDPVERNTVGVPAGGWTAIRFRADNPGVWFMHCHLEIHTTWGLKMAFVVDNGKGPNESLLPPPTDLPKC

SEQ ID NO: 87 - DNA, 1674 bp, Nicotiana tabacum
atgaactcttggattcgtcttttcatagtattggcagcttgtctttttcctcttgtcgttgaatgcaggattcgacattacaagttcaatgtggtaatgaagaacacgactcgcctttgttcatccaagcccattgttactgttaatggaaaatttccaggacctacaatctatgctcgggaaggtgacacagtacttgtcaaagttgttaaccatgtcaagtataatctctctatccattggcatggtattagacaacttagaacaggttgggcagatggaccagcatacatcacacaatgtccaattcagccagggcaaaactatgtgtataacttcactattacaggccaaaggggtacactattttggcatgctcatattttgtggctaagggccactgttcatggtgcaattgttatcttgcctaaccttggagtgccttatccattccctaaacccaaccacgaagctgtcgtgatcctagctgaatggtggaaatctgataccgaagctgtgattaatgaagccataaaatcaggattagcccctaatgtttctgatgctcacactattaatggtcatccgggacccgtctcaaattgcgcatcacaaggtggatacaaattgaacgttgatccaggaaaaacctacatgttacgagtcatcaacgctgcgctcaatgaagaactctttttcaaaattgctggccataaaatgacagtagttgaagttgatgccacttacattaaacctttcaaaacagacacaattgtaattgctcctggccaaacaacaaatgtaattgtcactgccaatcaaggttctggaaaatacatggttgctgcttcaccttttatggatgcaccaattgctgttgataatgttacagcaatagccactttacattattctggcacacaaggaaatagccacatttcacttactagtacaccacctaaaaatgccacccctgtagccaacacttttcttgattctttaagaagcctgaattccaaaaaataccctgctaaagttcccaaaaaaattgatcattccctatttttcacggtaggtttagggattaatccatgcccaacttgcaaacaaggtaatggaagcagagttgtggctagtgtaaacaatgttacattcgttatgccaacggttgcccttttacaagcacatttctttgggactaaaggagttttcacgacagattttccagcaaacccgccttttgctttcaactatacgggaacaggaccaactaatttggcgacgatgaatgggactaaggtttataggctgcggtataacgatacagttcaattggttttgcaggatactggaattatagcccctgagaaccatccaatccatttgcatggcttcaatttttttctagtgggtaaaggcataggaaattttaatccaaaaacagatcctaagaattttaatcttgtggatcctgttgagaggaatacagttggtgttcctgctggaggatgggttgctataagatttcgtgctgacaatccaggagtttggtttatgcattgtcatctagagatacacacaacatggggattgaaaatggcatggcttgtagataatggcaaaggcccaaatgagtcccttttgccacctcctaaggatcttccaaaatgctaa

SEQ ID NO: 88 - protein, 557 aa, Nicotiana tabacum
MNSWIRLFIVLAACLFPLVVECRIRHYKFNVVMKNTTRLCSSKPIVTVNGKFPGPTIYAREGDTVLVKVVNHVKYNLSIHWHGIRQLRTGWADGPAYITQCPIQPGQNYVYNFTITGQRGTLFWHAHILWLRATVHGAIVILPNLGVPYPFPKPNHEAVVILAEWWKSDTEAVINEAIKSGLAPNVSDAHTINGHPGPVSNCASQGGYKLNVDPGKTYMLRVINAALNEELFFKIAGHKMTVVEVDATYIKPFKTDTIVIAPGQTTNVIVTANQGSGKYMVAASPFMDAPIAVDNVTAIATLHYSGTQGNSHISLTSTPPKNATPVANTFLDSLRSLNSKKYPAKVPKKIDHSLFFTVGLGINPCPTCKQGNGSRVVASVNNVTFVMPTVALLQAHFFGTKGVFTTDFPANPPFAFNYTGTGPTNLATMNGTKVYRLRYNDTVQLVLQDTGIIAPENHPIHLHGFNFFLVGKGIGNFNPKTDPKNFNLVDPVERNTVGVPAGGWVAIRFRADNPGVWFMHCHLEIHTTWGLKMAWLVDNGKGPNESLLPPPKDLPKC

SEQ ID NO: 89 - DNA, 1665 bp, Vitis vinifera
atgacgcagtggattcgggtcttgcttctggtggcttctctgtttccgattttagtcgattgccggactcgtcactacaagttcaatgtggttttgagaaaggccacaaggttatgttcaagtaaacctatagtcactgtgaatgggaacttccctgggcctaccattcatgctagggaggatgatacagtgctgatcaaggttgtgaaccatgtcaagtataatgtcagcattcactggcatgggatcaggcagcttaggacaggttgggcggatgggccggcatatataacacagtgtccgattcagccggggcaaagctatatatacaatttcaccattacaggccaaaggggaacacttctatggcatgcgcacatcctctggctcagggctaccgtccatggcgccctcgtcatcttgcccaagcttggtgttccttatccatttcccactccctataaggaagctgttgttattttgggtgaatggtggaaatcagatgtggaagcagtgatcaatgaagctatgaagtctggcctggccccaaatgtgtccgatgctcacaccattaatggccatcctgggccattctcaagctgctccttacagggtgggtttacactgccagtagacccaggcaagacatacatgctgagaatcatcaacgctgcgctgaatgaagagctcttcttaaagattgcaggccatcatctgactgtggttgaggtcgacgccacttacactaaacccttttcaactgacaccgtcctcatcgctccggggcagaccaccaatgtactcctaacagcaggactcggcgctggaaagtacttggttacagcgtcttctttcatggattcccccattgcagtcgacaacatgactgcaacagccaccgttcaatactccggcaccctttccagtactccgactactatcaccaacccacctccccaaaacgccacctcagtggccacaaagttcacagattccctcagaagcctgaactcgaaaaagtacccagccagagttccattggccaccgatcactcccttctgttcactgttggacttgggattaacccctgtgctacttgcgttaatggcggtaaagtggtggctaatatcaacaatgtcacttttgtaatgcctactactgctcttcttcaagcccattacttcaaaatgaagggagtttacactgatgatttccctggaaaccccccaattgcattcaactatacaggcacacagcctacaaacttccagaccatgaatgggacaaggctttatagactggcatacaactctaccgttcagttagtcctacaagacaccggtatgatagcgcccgaaagacatccagtccatctccatggcttcaatttcttcgtcattggaaggggtttagggaactataacccaaagaaggacccaaaaaaattcaacctggttgaccctgttgagaggaacacagttggagtcccctctggtggatggactgccatcagattcagagcagataacccaggggtctggttcatgcactgccatttggaagtccacacaacatggggtctgaaaatggcttttgtggttgacaatggaaaaggccccaatgaatcagttctaccacctccaagtgaccttcccaaatgctaa

SEQ ID NO: 90 - protein, 554 aa, Vitis vinifera
MTQWIRVLLLVASLFPILVDCRTRHYKFNVVLRKATRLCSSKPIVTVNGNFPGPTIHAREDDTVLIKVVNHVKYNVSIHWHGIRQLRTGWADGPAYITQCPIQPGQSYIYNFTITGQRGTLLWHAHILWLRATVHGALVILPKLGVPYPFPTPYKEAVVILGEWWKSDVEAVINEAMKSGLAPNVSDAHTINGHPGPFSSCSLQGGFTLPVDPGKTYMLRIINAALNEELFLKIAGHHLTVVEVDATYTKPFSTDTVLIAPGQTTNVLLTAGLGAGKYLVTASSFMDSPIAVDNMTATATVQYSGTLSSTPTTITNPPPQNATSVATKFTDSLRSLNSKKYPARVPLATDHSLLFTVGLGINPCATCVNGGKVVANINNVTFVMPTTALLQAHYFKMKGVYTDDFPGNPPIAFNYTGTQPTNFQTMNGTRLYRLAYNSTVQLVLQDTGMIAPERHPVHLHGFNFFVIGRGLGNYNPKKDPKKFNLVDPVERNTVGVPSGGWTAIRFRADNPGVWFMHCHLEVHTTWGLKMAFVVDNGKGPNESVLPPPSDLPKC

SEQ ID NO: 91 - DNA, 1665 bp, Populus trichocarpa
atggcactatggcttcgtgttctagttctggtggcttgtctgtttccggcatctgtggagtccatggtccggcactacaagttcaatgtggtgatgaaaaataccacgagactgtgttcagaaaagcccattgtcactgtcaatggaaggttccctgggcccactttagttgccagggaagatgacacagtgctggtgaaggtggtcaaccacgtcaaatacaatgtcagtatccattggcatggaatcagacagctgcggacaggttgggccgatggcccagcatacataacgcagtgtcctcttcagccaggacagagcttcgtgtacaatttcaccatttctggtcaaaggggcaccctactctggcatgcacatatcctctggctcagggccactgtccatggtgctattgtcatcttgcccaagcgaggtgttccttacccatttcctacaccacacagggaagaagtcattgtattaggtgaatggtggaaatcagatgttgaagctgtgatcaacgaggcaatgaattctgggagggcacctaatgtctcagatgctcacacgatcaatggtcatccagggcctgtctcaggctgctcttcacagggaggatataatttaccagtacggcccggaaagacttacatgcttcggattatcaacgctgcactaaatgaagagctcttctttaagattgctggccatcaactcactgttgtagaggttgatgccacatacgttaaacccttcaaaatcgacaccattgtcatagccccaggccaaaccactaacgttcttgtgacagcaaaccgtggttctggcaagtacttggttgctgcctcacccttcatggacgcacccattgcggttgataacgtgacggccacagccacgttacattactctggcacccttgctagtaccaccactaccctcaccgtccctccagcgcaaaatgccaccccagtagctacaaactttacagatgctctacgtagcttgaattcgataaaatatcctgctcgagtcccattaaaaatcgatcactctctcttttttaccattggcctcggtgtcaatccttgtgctacttgtgtcaatgggaaccgagttgtggcagatatcaataacgttacatttgttatgccaacaattgctctccttcaagctcatttcttcaacatcaaaggtgttttcacagatgacttcccaggcaacccgcccacaccattcaactatacaggcacacaaccaaaaaactttcaaacagtgaatggaacaaaactttatagacttgcttataactctactgtccaactagtgctacaagatacaggaatgcttactcctgaaaaccaccctgtccatttgcatggcttcaatttcttcgaagttgggaggggaataggtaatttcaatccaaagagggacccaaagaaattcaatcttgctgacccagtggagaggaacacaattggagtcccagctggtggatggactgctataaggttcatagcagataatccaggggtttggttcatgcattgccatttggaagtccatacaacatggggacttaagatggcatttgttgtagacaatggcaaaggccctaatgaatctgttttacctcctcctccggatctaccaaagtgctag

SEQ ID NO: 92 - protein, 554 aa, Populus trichocarpa
MALWLRVLVLVACLFPASVESMVRHYKFNVVMKNTTRLCSEKPIVTVNGRFPGPTLVAREDDTVLVKVVNHVKYNVSIHWHGIRQLRTGWADGPAYITQCPLQPGQSFVYNFTISGQRGTLLWHAHILWLRATVHGAIVILPKRGVPYPFPTPHREEVIVLGEWWKSDVEAVINEAMNSGRAPNVSDAHTINGHPGPVSGCSSQGGYNLPVRPGKTYMLRIINAALNEELFFKIAGHQLTVVEVDATYVKPFKIDTIVIAPGQTTNVLVTANRGSGKYLVAASPFMDAPIAVDNVTATATLHYSGTLASTTTTLTVPPAQNATPVATNFTDALRSLNSIKYPARVPLKIDHSLFFTIGLGVNPCATCVNGNRVVADINNVTFVMPTIALLQAHFFNIKGVFTDDFPGNPPTPFNYTGTQPKNFQTVNGTKLYRLAYNSTVQLVLQDTGMLTPENHPVHLHGFNFFEVGRGIGNFNPKRDPKKFNLADPVERNTIGVPAGGWTAIRFIADNPGVWFMHCHLEVHTTWGLKMAFVVDNGKGPNESVLPPPPDLPKC

SEQ ID NO: 93 - DNA, 1671 bp, Populus trichocarpa
atggacatggcaccatggattcgggttctagttctggtggcttgtctgtttccggcatctgtggagtccatggtccggcactacaagttcaatgtggtaatgaaaaatagcacaaaactgtgttcaacaaagcccattgtcactgtgaatggacagttcccagggcctactttagttgccagagaagatgacactgtgcttgtgaaggtggtaaaccatgtcaaatacaatgtcagcatccattggcatggcattagacaactgcggaccggttgggccgatggccctgcatacataacgcagtgtcctattcagccagggcagagctttgtgtacaatttcaccattactggtcaaaggggcactctactctggcatgcacatatcctctggctcagggccactgtccatggtgctattgtaattttgcccaagcgaggtgttccatacccatttcctacaccacgcaaggaaaaagtcattatattaggtgaatggtggaaatcagatgttgaagctgtgatcaacgaggcgacgaaatccgggatagcacctaatgtctcggatgctcacacaatcaatggccatccagggcctgtctcagcctgctcttcacacggagggtataatttatcagtacatcccggaaagacttacatgcttcggatcatcaacgctgcactaaatgaagaactcttctttaagattgctggccatcaactcactgttgtagaggttgatgccacatacgtaaaacccttcaaaatcgacaccgttgtcatagccccgggccaaaccacaaacgttcttgtgacggcaaaccgtggttctggccagtacttggttgcagcctcacctttcatggacgcacccattgcggttgataacgtgacggccacagccaccttacattattctggcacccttgctagtaccatcactacactcaccgtacctcctgccaaaaacgccactccggtagcaacaaactttacgaatgctctacgtagcttgaattcgataaaatatcctgctagagtccctttaaagattgatcattctcttttcttcaccgttggccttggtgttaatccttgtgctacttgtatcaatggaagccgtgttgtggcagatatcaataacgttacatttgtgatgccaacaattgctctccttcaagctcacgtcttcaacattagtggtgttttcacagatgatttccctgccaacccgcccacgccattcaactatacaggcacacaaccaacgaactttcagacagtgaagggaacaaaactttatagacttgcttataacaacacagtccagctagttctacaagatacaggaatgcttactcctgaaaaccaccctgtccatttgcatggcttcaatttctttgaagttggaaggggagtaggtaatttcgatccaaataaggacccaaagaaattcaatcttgttgacccagtggagaggaacacaattggggtcccagctggtggatggaccgctataagattcatagcagataatccaggggtttggttcatgcattgccatttggaggtacacacaacatggggacttaagatggcatttgttgtagacaatggcaaaggcccaaatgaatctgttttaccaccgcctcctgatctgccaaagtgctag

SEQ ID NO: 94 - protein, 556 aa, Populus trichocarpa
MDMAPWIRVLVLVACLFPASVESMVRHYKFNVVMKNSTKLCSTKPIVTVNGQFPGPTLVAREDDTVLVKVVNHVKYNVSIHWHGIRQLRTGWADGPAYITQCPIQPGQSFVYNFTITGQRGTLLWHAHILWLRATVHGAIVILPKRGVPYPFPTPRKEKVIILGEWWKSDVEAVINEATKSGIAPNVSDAHTINGHPGPVSACSSHGGYNLSVHPGKTYMLRIINAALNEELFFKIAGHQLTVVEVDATYVKPFKIDTVVIAPGQTTNVLVTANRGSGQYLVAASPFMDAPIAVDNVTATATLHYSGTLASTITTLTVPPAKNATPVATNFTNALRSLNSIKYPARVPLKIDHSLFFTVGLGVNPCATCINGSRVVADINNVTFVMPTIALLQAHVFNISGVFTDDFPANPPTPFNYTGTQPTNFQTVKGTKLYRLAYNNTVQLVLQDTGMLTPENHPVHLHGFNFFEVGRGVGNFDPNKDPKKFNLVDPVERNTIGVPAGGWTAIRFIADNPGVWFMHCHLEVHTTWGLKMAFVVDNGKGPNESVLPPPPDLPKC

SEQ ID NO: 95 - DNA, 1677 bp, Arabidopsis thaliana
atggggtctcatatggtttggtttctatttcttgtatccttcttctctgtgttcccagctccatctgagagcatggttcgccactacaagtttaacgttgtaatgaagaacgtgactagattatgctcaagcaagccaaccgtgaccgtcaacggtagatatccaggtcccacaatctacgcacgagaagatgacacgttgctcatcaaagtcgttaatcacgtcaagtacaacgtctccatccactggcacggtgtgagacaagtgagaacgggatgggctgatgggcctgcttacataactcagtgcccgatccagcctggtcaagtctacacatacaactacactttgaccggccaacgcggaacgctctggtggcacgctcatatcctctggctccgagccactgtttacggtgcattggtcatccttcccaaacgcggtgttccctatcctttccccaaacccgacaatgagaaagtcatcgttctaggtgaatggtggaaatcggatactgaaaatattattaatgaggcgcttaagtctggattagcccctaatgtctctgactctcacatgatcaacggacacccaggcccagttagaaactgtccatctcaaggttacaaactgtcagtagagaatggcaaaacctatctgctacgactagtcaacgctgcacttaatgaagaactctttttcaaagtcgccggccatattttcacggtggtagaagtagacgcagtctatgttaaaccgttcaagaccgacaccgtccttatagcccccggtcaaaccaccaacgtcctcctaaccgcctcaaaatccgccgggaaataccttgtaaccgcttctcctttcatggacgccccaatcgcggtggacaacgtaaccgccaccgcaactgttcattactcgggaacactctcctcctccccaacaatcctcacccttcctcccccgcaaaacgctacttccatagccaacaacttcacaaactctcttcgtagtctcaactccaagaagtaccctgctcttgtcccgaccaccatcgaccaccacctcttcttcaccgtcggccttgggctaaacgcatgccctacttgcaaggccggaaacggaagccgtgtcgtggctagcatcaacaatgtaaccttcattatgcctaaaaccgctttgctcccggctcattacttcaacacaagtggagttttcacgacagactttcccaagaatccaccacacgttttcaactacagcggaggatcagtcacgaacatggccacagaaaccggcacaaggctctacaagctaccgtataacgccactgttcagcttgtccttcaagataccggcgtcatagcgccagagaaccatccagtacatcttcacggttttaacttttttgaagtcggtcgtggattaggtaacttcaactccacgaaagacccaaaaaacttcaatttggtagatccggttgagaggaacacaatcggagttccatccggtggatgggtcgtcatcagattcagagcagataatcccggggtttggttcatgcattgtcacttggaggtacacacgacgtggggattaaagatggctttcttggtggagaacggcaaaggacccaatcagtcgattttgccgccgcctaaggatcttcccaagtgctaa

SEQ ID NO: 96 - protein, 558 aa, Arabidopsis thaliana
MGSHMVWFLFLVSFFSVFPAPSESMVRHYKFNVVMKNVTRLCSSKPTVTVNGRYPGPTIYAREDDTLLIKVVNHVKYNVSIHWHGVRQVRTGWADGPAYITQCPIQPGQVYTYNYTLTGQRGTLWWHAHILWLRATVYGALVILPKRGVPYPFPKPDNEKVIVLGEWWKSDTENIINEALKSGLAPNVSDSHMINGHPGPVRNCPSQGYKLSVENGKTYLLRLVNAALNEELFFKVAGHIFTVVEVDAVYVKPFKTDTVLIAPGQTTNVLLTASKSAGKYLVTASPFMDAPIAVDNVTATATVHYSGTLSSSPTILTLPPPQNATSIANNFTNSLRSLNSKKYPALVPTTIDHHLFFTVGLGLNACPTCKAGNGSRVVASINNVTFIMPKTALLPAHYFNTSGVFTTDFPKNPPHVFNYSGGSVTNMATETGTRLYKLPYNATVQLVLQDTGVIAPENHPVHLHGFNFFEVGRGLGNFNSTKDPKNFNLVDPVERNTIGVPSGGWVVIRFRADNPGVWFMHCHLEVHTTWGLKMAFLVENGKGPNQSILPPPKDLPKC

SEQ ID NO: 97 - DNA, 1671 bp, Vitis vinifera
atgaagtactcttgggttcgcgttctggttcttgtggcttgcctgttaccagcattagtcgagtgcagggttcggcaatacacatttaatgtggtgttgaagaatacaaatagactatgcgctagcaagcccattgtcaccgtgaatggacggttcccggggcccactctctatgtcagggaagacgacacagtgcttgtcagggttgtcaaccacgtcaaatacaatgtttccatccattggcatggtataaggcaactgagaacggggtggtcggacgggccagcatacatcacccagtgcccaatccagacagggcaaagctatttatacaacttcaccgttactggccaacggggcacacttctttggcatgcgcatgtcctttggctaagggcaactgtccatggtgccatcgttatcttgcctaagcgaggggtgccctatccttttccaaaaccccataaggaggttgttgtcatattaggtgagtggtggaaatcagatgtcgaggctgtgatcaatcaagctcttagctccggtttggctcccaatgtttcggatgctcacaccattaacggccatccaggccctgtctccaattgtccttcacagggcggcttttcattgcaagtcgaggccggaaaaacctacctactccgaatcattaacgctgcactcaatgaagaactcttcttcaagattgctggacatcaactctctgtcgtagaagtggacgccgtctacacaaagccattcaagatcgacactcttgttattgcccctggccaaaccacaaatgctcttatcacagcagatcaaagctctggcaagtacttgatggccgcctcccctttcatggactcccccatcgcagtagataaccttactgcaacagcaactctgcactactccggcacacttgcgagttcccccaccagtcttactagcccacctccaccaaatgcaactgcagttgctaacaagttcgtaaactctctccgaagccttaattccaaaaagtacccagccaaagttccattaaccgtggatcactcccttttcatcacggtggggctaggaattaacccttgtgctacctgcgtcgccggtaatggaagccgggcagtggctagtatgaataatgtcacgtttgtgatgccaactacagccattcttcaagcacactacttcaacataagcggtgtgtacaccaccgacttcccagctaacccacccgtggcttttaattacactggctcgcccgcaaacttgcagaccacgaatgggacaaaggtttatagggtaccttacaactctacggttcaagtagttttacaagatactgggatcatcgccactgagaaccatccagtccatctacatgggttcaacttcttcgctgtgggcaaaggactgggaaattacaacccaaagacggatcctcaaaactttaatctcattgatcctgttgagcggaacactattggagtaccatccggtgggtggacagctatcagatttctagcagataatccaggagtttggttcatgcattgccacttggaagtgcatacaacctggggactgaagatggcattcttggtggacaacggcaaaggccccaaccagtccattataccccctccaagtgatcttcccaaatgttaa

SEQ ID NO: 98 - protein, 556 aa, Vitis vinifera
MKYSWVRVLVLVACLLPALVECRVRQYTFNVVLKNTNRLCASKPIVTVNGRFPGPTLYVREDDTVLVRVVNHVKYNVSIHWHGIRQLRTGWSDGPAYITQCPIQTGQSYLYNFTVTGQRGTLLWHAHVLWLRATVHGAIVILPKRGVPYPFPKPHKEVVVILGEWWKSDVEAVINQALSSGLAPNVSDAHTINGHPGPVSNCPSQGGFSLQVEAGKTYLLRIINAALNEELFFKIAGHQLSVVEVDAVYTKPFKIDTLVIAPGQTTNALITADQSSGKYLMAASPFMDSPIAVDNLTATATLHYSGTLASSPTSLTSPPPPNATAVANKFVNSLRSLNSKKYPAKVPLTVDHSLFITVGLGINPCATCVAGNGSRAVASMNNVTFVMPTTAILQAHYFNISGVYTTDFPANPPVAFNYTGSPANLQTTNGTKVYRVPYNSTVQVVLQDTGIIATENHPVHLHGFNFFAVGKGLGNYNPKTDPQNFNLIDPVERNTIGVPSGGWTAIRFLADNPGVWFMHCHLEVHTTWGLKMAFLVDNGKGPNQSIIPPPSDLPKC

SEQ ID NO: 99 - DNA, 1680 bp, Glycine max
atggactcttgggttagaatgctgtttctcattgcttgcattgttccagctttagtcgagtgcaaagtgaggcactacaaattcaatgtggtactgaagaatactactaggttatgttcaagcaaaccaattgtcaccgttaatgggaaatttccaggacccactctttacgctagggaagatgatacagtgctagtcaaagtcaataaccttgtcaaccacaatgtcaccatccactggcatggtgtgagacaacttagaactggttgggctgatgggcctgcatacatcacacagtgcccaattctgtcaggacaatcatatttgtacaacttcacccttacaggacaaagaggaacacttctttggcatgctcatgttaactggctaaggtcaactctccatggtgccatagttatcttgccaaagaggggtgtgccataccctttcccaaaaccagataaagagttggttgtaatattaggagaatggtggaaatctgatacagaagatgttatcaatgaagctctcaagtcaggattagcaccaaatgtctcagatgctcataccattaatggccttccagggattgtgtctgtggccaattgttctacacaagatgtgtacaagcttcctgtggaaagtggcaagacctacctattgagaatcatcaatgctgcactcaatgaggagctcttcttcaaaattgctggccatccattcactgtggttgaagttgatgcctcatatgtaaagcccttcaagacagacactctttcgatagcccctggtcaaaccaccaatgcccttttaactgctgaccaaaactctggcaaatacacaattgtagcctctactttcatggattctccagttgtagctgtggataacttgactgcaacagccacattgcactacactggcactcttgccaccactcctacacttctcaccacccctcctccaagaaatgccacccaagttgctaacaacttcaccaattctctcaaaagccttaattctaagaaatacccagcaaaagtcccacaaaaagttgatcattcacttctcctcactgttggtttggggattaacccttgtccatcttgcacagctggcaatgggagtagagtagtggctgctgtcaacaatgtgacatttgtgatgccaaccactgcactgcttcaggctcattacttcaacatcaagggggtgttcaccactgatttccctggcaacccttctcatgtttacaactacacggcgactccgccggcggcagcttggcagaccacaaatggcaccaaggcttacaggctggcattcaactccacagttcaggttgttttgcaggatactggggtcattgctcctgagagccacccggttcatcttcatgggttcaacttctttgttgttggctctggtgtaggaaattatgatcccaaaacggatcaaaataactttaatcttgcggatcctgttgagaggaacacaattggagtgcctactggagggtgggttgctttcagattcagagcagataacccagtgtggttcttgcactgccatttcgaggtgcatacaacatgggggttgaagatggcattcttggtggacaatggtaaaggtcctaatgaatcactgctacccccaccaaaagatcttccaaaatgttag

SEQ ID NO: 100 - protein, 559 aa, Glycine max
MDSWVRMLFLIACIVPALVECKVRHYKFNVVLKNTTRLCSSKPIVTVNGKFPGPTLYAREDDTVLVKVNNLVNHNVTIHWHGVRQLRTGWADGPAYITQCPILSGQSYLYNFTLTGQRGTLLWHAHVNWLRSTLHGAIVILPKRGVPYPFPKPDKELVVILGEWWKSDTEDVINEALKSGLAPNVSDAHTINGLPGIVSVANCSTQDVYKLPVESGKTYLLRIINAALNEELFFKIAGHPFTVVEVDASYVKPFKTDTLSIAPGQTTNALLTADQNSGKYTIVASTFMDSPVVAVDNLTATATLHYTGTLATTPTLLTTPPPRNATQVANNFTNSLKSLNSKKYPAKVPQKVDHSLLLTVGLGINPCPSCTAGNGSRVVAAVNNVTFVMPTTALLQAHYFNIKGVFTTDFPGNPSHVYNYTATPPAAAWQTTNGTKAYRLAFNSTVQVVLQDTGVIAPESHPVHLHGFNFFVVGSGVGNYDPKTDQNNFNLADPVERNTIGVPTGGWVAFRFRADNPVWFLHCHFEVHTTWGLKMAFLVDNGKGPNESLLPPPKDLPKC

SEQ ID NO: 101 - DNA, 1674 bp, Medicago truncatula
atggtttcttggattcgagtactacttcttatttgttgcatatttccagcgttggtcgagtgcaaagttaggcactacaaatttcatgtggtggccaaaaatacaagtagattatgttcaagcaaagcaattgtcacagttaatgggaagtttccgggacccactctttatgccagggaagatgatacagtgatagtcaaagttcgtaatcaagtcaacaacaatattaccatccattggcatggtattaggcaattgagaacaggttgggctgatggacctgcatatatcacacaatgtccaattcaacctggacatagttatacatacaatttcaccattactggtcaaagaggaacacttctttggcatgcacatgttaattggctaagatcaactgtccatggtgccattgttattttaccaaagaaaggtgttccttatcctttcccaaagccagatgatgaattggttttggtattaggagagtggtggaaatctgatactgaagctgttatcaatgaagctctaaaatcaggattggcaccaaatgtttcagatgctcataccataaatggtcttcctgggacagtggccaattgttctacacaagatgtttacaaactacctgtggagagtggcaaaacctatctactaagaatcatcaatgctgcactcaatgaagaacttttcttcaaaatagctggccacaaactcacagtagtagaagttgatgcaacatacacaaaaccattccaaattgaaacaattgtgatagcacctggtcaaacaacaaatgtactattaaaagcaaaccaaaaatctggcaaatatttagtagcagcttcacctttcatggatgcacctgttgctgtagacaatttaacagccacagctacattacattattcaggaactactcttacaaacacccctacatttctaactacacctccacctacaaatgctacacaaattgctaataactttttaaactcacttaaaggacttaattccaagaaatacccagttaatgttcctttaaaaattgatcattcacttttttttactgttggattaggggttaatccttgtccttcatgtaaagctggaaatggtagtagagttgtagctgctattaataatgttacatttgttatgccaactacagctttactacaagcacattatttcaacattaagaatgtttttactgcagattttccaccaaatccacctcatatttataatttcactggtgctggacctaaaaacttgaacactacaagtggtacaaagttatataagttatcatttaatgatactgttcagcttgttatgcaagatactggaatcattgcacctgagagtcatcctgttcatcttcatggtttcaatttctttgttgttggtagaggagttggaaattatgatagtaaaaatgattcaaagaagtttaaccttgttgatcctgttgaaaggaacactgttggtgtaccagctggtggttgggttgccattagattcagagctgataatccaggagtttggttcatgcattgccatttggaggtgcatacaacatggggtctaaagatggcctttttggtggacaatggcaaaggtcctaaacaatcagtgatagctccacctaaagatcttccaaaatgttga

SEQ ID NO: 102 - protein, 557 aa, Medicago truncatula
MVSWIRVLLLICCIFPALVECKVRHYKFHVVAKNTSRLCSSKAIVTVNGKFPGPTLYAREDDTVIVKVRNQVNNNITIHWHGIRQLRTGWADGPAYITQCPIQPGHSYTYNFTITGQRGTLLWHAHVNWLRSTVHGAIVILPKKGVPYPFPKPDDELVLVLGEWWKSDTEAVINEALKSGLAPNVSDAHTINGLPGTVANCSTQDVYKLPVESGKTYLLRIINAALNEELFFKIAGHKLTVVEVDATYTKPFQIETIVIAPGQTTNVLLKANQKSGKYLVAASPFMDAPVAVDNLTATATLHYSGTTLTNTPTFLTTPPPTNATQIANNFLNSLKGLNSKKYPVNVPLKIDHSLFFTVGLGVNPCPSCKAGNGSRVVAAINNVTFVMPTTALLQAHYFNIKNVFTADFPPNPPHIYNFTGAGPKNLNTTSGTKLYKLSFNDTVQLVMQDTGIIAPESHPVHLHGFNFFVVGRGVGNYDSKNDSKKFNLVDPVERNTVGVPAGGWVAIRFRADNPGVWFMHCHLEVHTTWGLKMAFLVDNGKGPKQSVIAPPKDLPKC

SEQ ID NO: 103 - DNA, 2423 bp, Oryza sativa
atggccgtcctgccggagtcacgccggctctccctgctcctcatggccgcctgcttcctgcttcaggctctcagcgcccacgccatcacccggcattacaagttcaatgtgaggatatatcgtcatcaaactttgccgcatgataactgcaagcatgcatcgcaattgtcacatacatatacaggttgcagcagaaatggcaagatggtttattaccacggttttgtctgtcttttctgtgtcttgtgcaggtggtcatgcggaacatgacacggctttgctcaaccaagcctatcctcactgtcaatggcaagttcccggggccaaccctgtatgcaagagaaggcgataatgtcctagtcaaggtcgtcaatcatgtagctcacaatgttaccatccactggtaagcagcttagctagctagcccatggctactccatgcatatgcttcaactccacgtgtaatttgcaccacaatttgaatcaccacctcaaggctcctttgctctctgtaagtacccaaaaagaaaaggagaatatcatgctgtgttagttaaaaggtaaaaagtatgtatagcttgcattagatcctctctgttgaacagaaaacagaaatgtgctagttattgttcccatttatgcaacaaaaatacacttggtgaataacttgataaacaagcatgtcatactaatcaggcacggggtaaggcagatccggacagggtggtacgatgggccagcttacataacacagtgcccaatacagccaggaagcagctttctctataatttcactataacagggcaacgaggcacactcctctggcatgctcacatcaattggctaagggctaccgtccatggtgctatcgtcatccttccaaagcttggcgtgccctaccccttccctgcacctcacaaggaggctgttatcgtgttaggtatatatatacacacacatatatatagctgcttctctacatcttgatgcatccagcacttaatttatcaagtgctgcatgcaggagaatggtggaaagaggacacagaaactgtaattaaccaggccatgcagctaggagtgggacccaatatttctgattcacacaccatcaatggtcatcctggtccgctgtctgagtgtgcttcttcacaaggtaatactcttactctacatgaacacgagacagacagtgcatgttatggctaactatgtgctagtcctacaactgagccaaagacctaacgatttcctgatgtatgcttgcaatacaatgcacagatgggttcaagctcagcgtcgaaaatggcaaaacatacatgctccggatcatcaacgctgcactgaatgatgacctatttttcaaggttgctgggcacgaattaactgtggttgaggttgatgcagtctataccaagccatttaagaccgataccctgctcatcactccaggccagaccaccaacgtccttgttagagctaaccaaggcgctggccgttaccttctctcagtctcccccttcatggatgcgcccgtgcaggttgacaacaaaacaggcaccgctactttgcactacgccaacacagtctcgtcaagcatggcatccctcactctcgtcaagccaccaccacaaaatgccacccatatcgtatcaaaatttactgattcactccatagtctcaattccaaggagtacccagccaatgtgccacagacagtagaccactcccttctccttaccgttggcgtaggtgtcaatccatgtccaagctgcatcaatgggacaagggtggtgggcacaatcaacaacgtgacgttcatcatgccatcaaccccaattcttcaagcccactattacaacattccaggagtgttcacagaagacttcccagcaaccccactgcacaagttcaactatacaggaagtggtcccaaaaaccttcaaaccatgaatggaaccagggtttacaggctgccatacaatgcttcagtgcaggttgtactccaggatacgggaatcatatcaccagaaagccacccaatccatttacatggctttaatttttttgtggttgggaaaggggtcgggaactacaaccccaggacctccccttctacattcaacctcatcgaccccatcgagaggaacaccattggagttcctactggtggttggacggcaatcagatttaggtcagacaacccaggtattgattttaaaacctatgtttttttaagataaataaccccttctgtttccagacacattgattgagctgttatattgcaggtgtttggtttatgcattgtcatttcgaggtgcacacatcatggggactcaagatggcctttgtggtggataatggaaagagacctagtgagactctgattccaccacccaaagatcttccgcagtgttaa

SEQ ID NO: 104 - protein, 564 aa, Oryza sativa
MAVLPESRRLSLLLMAACFLLQALSAHAITRHYKFNVVMRNMTRLCSTKPILTVNGKFPGPTLYAREGDNVLVKVVNHVAHNVTIHWHGVRQIRTGWYDGPAYITQCPIQPGSSFLYNFTITGQRGTLLWHAHINWLRATVHGAIVILPKLGVPYPFPAPHKEAVIVLGEWWKEDTETVINQAMQLGVGPNISDSHTINGHPGPLSECASSQDGFKLSVENGKTYMLRIINAALNDDLFFKVAGHELTVVEVDAVYTKPFKTDTLLITPGQTTNVLVRANQGAGRYLLSVSPFMDAPVQVDNKTGTATLHYANTVSSSMASLTLVKPPPQNATHIVSKFTDSLHSLNSKEYPANVPQTVDHSLLLTVGVGVNPCPSCINGTRVVGTINNVTFIMPSTPILQAHYYNIPGVFTEDFPATPLHKFNYTGSGPKNLQTMNGTRVYRLPYNASVQVVLQDTGIISPESHPIHLHGFNFFVVGKGVGNYNPRTSPSTFNLIDPIERNTIGVPTGGWTAIRFRSDNPGVWFMHCHFEVHTSWGLKMAFVVDNGKRPSETLIPPPKDLPQC

SEQ ID NO: 105 - DNA, 1380 bp, Glycine max
atggcgtttgctcacgacttggagttggaatcctcttcttcccctctggtgttctcagtacggaggagtgaaccggagctggtggctccggccaagcccactccccgcgaaattaagatcctatctgagatagattctcaggcgggtcttcgtacccaaattccaatcatacagttctaccgtaacgacccatcattggcaggcaaggacccagttcaagccattcgaaacgctcttgcggaagcacttgttttctactacccgtttgcgggtagaattaaggaagaaggttcggatggaaaactcgtggtggattgtaacgaagagggtgtcatgttcatcgaggctgacgctgatgtcacacttgatcagttcggtgatgctctgaaacctccgtttccgtgcttccaagaactcctttaccaacctcctggttcagacggaatcactgatgcccccatttttcttatacaggtgacccgtttgaagtgcggtggtttcatcttggcaattcgtttcaaccacgtcatggttgacggagttgggctcattcatttcacactcaccgtggcagggatagcacgcggagccatgaaggagcctccattccaacccgtgtggagcagggagcttctcttcgccagggatccgccacgtgtcaccttcaaccaccgcgagtacgaacaactcacagactccaacgatgccgtttcgacagacttcgagcagcgttccttcttcttcggccccaccgaaaccgcctccatccgcgccctcctcccccgtgacctcgaccaacgcgccacaacgttcgaggtgctgacgtcatacgtctggcgctgccgcacgaaagcgcttcaaatccctccaaatgaagatgtgagaatgatgtgcatcgtcgacgcgcgtggaaagttcgatcctcccttcccggcggggttctacggcagctgcttcgcgtttccggcggcggtgacgggcgccggagatctctgcgagaaaccgctggagtacgcggtgcagttgatacagaaagcgcgcggcgaggtgagcgaagagtatattgattcggtggcggatttgatggcgagtgagggaaggcctttgttcactgtggtgaggtcgtgtctggtgttggatacgacggaggctgggttcagaaatttggactttggttgggggaatgcgctgtatggtggaatggcagttgctggtgctggtgctttccctgcggtgaactttcatgtgccgagtcagaatgccaagggcgaagaaggaattctggttttgatttgcttgcctagccaagtcatgaaagcctttgctaaagagttggatcataacatgcatgcttcagaagcaacttcattaattaactaa

SEQ ID NO: 106 - protein, 459 aa, Glycine max
MAFAHDLELESSSSPLVFSVRRSEPELVAPAKPTPREIKILSEIDSQAGLRTQIPIIQFYRNDPSLAGKDPVQAIRNALAEALVFYYPFAGRIKEEGSDGKLVVDCNEEGVMFIEADADVTLDQFGDALKPPFPCFQELLYQPPGSDGITDAPIFLIQVTRLKCGGFILAIRFNHVMVDGVGLIHFTLTVAGIARGAMKEPPFQPVWSRELLFARDPPRVTFNHREYEQLTDSNDAVSTDFEQRSFFFGPTETASIRALLPRDLDQRATTFEVLTSYVWRCRTKALQIPPNEDVRMMCIVDARGKFDPPFPAGFYGSCFAFPAAVTGAGDLCEKPLEYAVQLIQKARGEVSEEYIDSVADLMASEGRPLFTVVRSCLVLDTTEAGFRNLDFGWGNALYGGMAVAGAGAFPAVNFHVPSQNAKGEEGILVLICLPSQVMKAFAKELDHNMHASEATSLIN

SEQ ID NO: 107 - DNA, 1380 bp, Glycine max
atggcgtttgctcacgacttggagttggaatcctcttcttcccctctggtgttctcagtacggaggagtgaaccggagctggtggctccggccaagcccactccccgcgaaattaagatcctatctgagatagattctcaggcgggtcttcgtacccaaattccaatcatacagttctaccgtaacgacccatcattggcaggcaaggacccagttcaagccattcgaaacgctcttgcggaagcacttgttttctactacccgtttgcgggtagaattaaggaagaaggttcggatggaaaactcgtggtggattgtaacgaagagggtgtcatgttcatcgaggctgacgctgatgtcacacttgatcagttcggtgatgctctgaaacctccgtttccgtgcttccaagaactcctttaccaacctcctggttcagacggaatcactgatgcccccatttttcttatacaggtgacccgtttgaagtgcggtggtttcatcttggcaattcgtttcaaccacgtcatggttgacggagttgggctcattcatttcacactcaccgtggcagggatagcacgcggagccatgaaggagcctccattccaacccgtgtggagcagggagcttctcttcgccagggatccgccacgtgtcaccttcaaccaccgcgagtacgaacaactcacagactccaacgatgccgtttcgacagacttcgagcagcgttccttcttcttcggccccaccgaaaccgcctccatccgcgccctcctcccccgtgacctcgaccaacgcgccacaacgttcgaggtgctgacgtcatacgtctggcgctgccgcacgaaagcgcttcaaatccctccaaatgaagatgtgagaatgatgtgcatcgtcgacgcgcgtggaaagttcgatcctcccttcccggcggggttctacggcagctgcttcgcgtttccggcggcggtgacgggcgccggagatctctgcgagaaaccgctggagtacgcggtgcagttgatacagaaagcgcgcggcgaggtgagcgaagagtatattgattcggtggcggatttgatggcgagtgagggaaggcctttgttcactgtggtgaggtcgtgtctggtgttggatacgacggaggctgggttcagaaatttggactttggttgggggaatgcgctgtatggtggaatggcagttgctggtgctggtgctttccctgcggtgaactttcatgtgccgagtcagaatgccaagggcgaagaaggaattctggttttgatttgcttgcctagccaagtcatgaaagcctttgctaaagagttggatcataacatgcatgcttcagaagcaacttcattaattaattaa

SEQ ID NO: 108 - protein, 459 aa, Glycine max; identical to SEQ ID NO: 106

SEQ ID NO: 109 - DNA, 1413 bp, Glycine max
atggctgagaatgcaattcatgtagtgatgcttccatggtcggcctttggccacttaataccatttttcaaactttccatagccttagccaaagctggtgttcatgtctcctttatatccactcccaaaaacattcaaaggcttcccaaaattccttcaaatttagctcatttggttgatttggtgcaatttcctttgccatcattagacaaagagcacttgccagaaggtgctgaggccactgtagacattccatctgaaaaaattgagtacttaaagttggcatatgataaattgcaacatgctgtcaaacaatttgtggccaatcagttgccaaattggataatttgtgattttagtcctcactggatagtagacattgttcatgagtttcaggtaaagttgatcttctataatgttttatctgctcctgcattaacagtttggggaccaccaggtacaaggaaaactcccttgtctccagaaagtttaacagcaccaccagaatgggtgacatttccatcttcagtggcttatcgaatacacgaggcaatagcgctttgtgccggtgccaacccggtaaatgcttctggggtaagtgattttgaaaggcttcacaaggtatttaatgcctctgaagctgtaatttttcgtagctgctacgagattgaaggagaatatctcaatgcataccagaagctagttgggaagcctgtgattcctataggtttattgcctgcagatagtgaagaaagagggagagaaattattgatgggagaactagtggtaagatatttgagtggcttgatgagcaagcatcaaaatctgttgtatttgtggggtttggcagtgagttgaagctgaacaaggatcaagtttttgagatagcttatggaattgaggagtatgaattgccatttatatgggcactcagaaaaccaagttgggcaatcaatgatgaagattttctaccttttggttttattgaaaggacatctaacagaggagttgtttgtatggggtggataccacagcaagaaatattggcacatccatctattggagggtctttgtttcactctggttggggttctgtcattgaaaccctacaatttggtcatatcctagttgtattacccttcattatagatcaacctcttaatgcaaggtttttggttgaaaagggtctagccattgaagtgaaaaggaatgaagatggatcattcactagaaatgacattgccacatcccttagacaagctatggtattggaggaaggtaagaagatcagaattaatacaggagaagctgctgcaattgtaggaaacttgaagctgcaccaagatcattacattgctgaatttgttcagtttcttaagaatggaatccggaaagtggaaacagatgtgtag

SEQ ID NO: 110 - protein, 470 aa, Glycine max
MAENAIHVVMLPWSAFGHLIPFFKLSIALAKAGVHVSFISTPKNIQRLPKIPSNLAHLVDLVQFPLPSLDKEHLPEGAEATVDIPSEKIEYLKLAYDKLQHAVKQFVANQLPNWIICDFSPHWIVDIVHEFQVKLIFYNVLSAPALTVWGPPGTRKTPLSPESLTAPPEWVTFPSSVAYRIHEAIALCAGANPVNASGVSDFERLHKVFNASEAVIFRSCYEIEGEYLNAYQKLVGKPVIPIGLLPADSEERGREIIDGRTSGKIFEWLDEQASKSVVFVGFGSELKLNKDQVFEIAYGIEEYELPFIWALRKPSWAINDEDFLPFGFIERTSNRGVVCMGWIPQQEILAHPSIGGSLFHSGWGSVIETLQFGHILVVLPFIIDQPLNARFLVEKGLAIEVKRNEDGSFTRNDIATSLRQAMVLEEGKKIRINTGEAAAIVGNLKLHQDHYIAEFVQFLKNGIRKVETDV

SEQ ID NO: 111 - DNA, 1386 bp, Glycine max
atggctgagaatccgattcgtgtaacgatgattccatggtctgcctttggccacttgataccatttttcaaactttccatagccttagccaaagctggtgttcatgtctccttcatatccactcccaaaaacattcaaaggcttcccaaaattccttcgactttatctcatttggttcattttgtggaacttcctttaccatcattggacaatgatatcttgccagaaggtgctgaggccaccgtggacattccatttgaaaagcatgagtacttaaaggcagcatttgataagctccaagatgctgtgaagcaatttgtagccaatcagttaccagattggataatctgtgatttcaatcctcactgggttgtagacattgcccaagagtttcaggtaaagttaatcttattttctattttatctgctacaggaacaacattcataggaccaccaggtacaagggcaggtcacttatctccagaaagtctaacagctccaccagaatgggtgacatttccatcttcagtggcttttcgaatacacgaggcaatacatttctgtgctggattcgacaaggtaaattcttccggggtaagtgatttcgaaagggttataaagatacatgatgcctctaaagctgtgatatttcgtagctgctacgagattgaaggagaatatctgaatgcatatcagaaactatttgagaagcctatgattccaatcggtttattgcctgtagagagaggagttgttgatgggtgtagtgataacatatttgagtggcttgataaacaagcatcaaaatcagttgtgtttgtggggtttggcagtgagttaaagctgagcaaggatcaagtttttgagatagcttatggacttgaggaatctcaattgccgtttttatgggcactgagaaaaccaagttgggaaagcaatgatgggtattctctacctgttggttttattgaaaggacatctaacagaggaagagtttgtaagggatggataccacaactagaaatattggcacattcatctattggaggatctttgtttcactctggctggggttctgtcattgaaaaccttcagtttgggaatacacttgttttattaccctttaatatagagcaacctcttaatgcaaggtttttggtggaaaagggattagccattgaagtgaaaagaaatgaagatgggtcattcactagaaatgacattgccgcatcccttagacaagccatggtattggaggaaggaaagaagatcagaaataatacaagagaagctgctgcaattgtaggaaacttgaagctgcaccaggatcactacgtagctgcatttgtacagtttcttaagaatggaatcaggaaaccaatatga

SEQ ID NO: 112 - protein, 461 aa, Glycine max
MAENPIRVTMIPWSAFGHLIPFFKLSIALAKAGVHVSFISTPKNIQRLPKIPSTLSHLVHFVELPLPSLDNDILPEGAEATVDIPFEKHEYLKAAFDKLQDAVKQFVANQLPDWIICDFNPHWVVDIAQEFQVKLILFSILSATGTTFIGPPGTRAGHLSPESLTAPPEWVTFPSSVAFRIHEAIHFCAGFDKVNSSGVSDFERVIKIHDASKAVIFRSCYEIEGEYLNAYQKLFEKPMIPIGLLPVERGVVDGCSDNIFEWLDKQASKSVVFVGFGSELKLSKDQVFEIAYGLEESQLPFLWALRKPSWESNDGYSLPVGFIERTSNRGRVCKGWIPQLEILAHSSIGGSLFHSGWGSVIENLQFGNTLVLLPFNIEQPLNARFLVEKGLAIEVKRNEDGSFTRNDIAASLRQAMVLEEGKKIRNNTREAAAIVGNLKLHQDHYVAAFVQFLKNGIRKPI

SEQ ID NO: 113 - DNA, 1386 bp, Glycine max
atggctgagaatccaattcgtgtaacgatgattccatggtctgcctttggccacttgataccatttttcaaactttccatagccttagccaaagctggtgttcatgtctccttcatttccactcccaaaaacattcaaaggcttcccaaaattccttcgactttatctcatttggttcattttgtggaacttcctttaccatcattggacaatgatatcttgccagaaggtgctgaggccaccctggacattccatttgaaaaacatgagtacttgaaggcagcatatgataagctccaagatgctgtgaagcaatttgtggccaatcagttaccagattggataatctgtgattttaatcctcactgggtggtagacattgcccaagagtttcaggtaaagttaatcttgtttgttattatatctgctactggagcaacattcataggaccaccaggtacaaggacaggtcccttatctccagaaagtctaacagctccaccagaatgggtgacatttccatcttcagtggcttttcgaaaacacgaggcaatacatttctgtgctggttcctacaaggtaagttcttccggggtaagtgatttcgaaaggattataaagttacatggtgcctctaaagctgtgttatttcgtagctgctacgagattgaaggagaatatctgaatgcatttcagaaactagttgagaagcctgtgattccaattggtttattgcctgtagagagacaagttgttgatgggtgtagtgataccatatttgagtggcttgataaacaagcatcaaaatcagttgtgtttgtggggtttggcagtgagttaaagctgagcaaggatcaagtttttgagatagcttatggacttgaggaatctcaattgccgtttttatgggcactgagaaaaccaagttgggaaagcaatgatgaatattctctacctgttggttttattgaaaggacttctaacagaggaagtgtttgtaagggatggataccacaactggaaatattggcacattcatctattggaggatctttgtttcactctggcttgggttctgtcattgaaaaccttcagtttgggcatacacttgttgtattacccttcaatatagatcaacctcttattgcaaggtttttggtggaaaagggactagccattgaagtgaaaagaaatgaagatgggtcattcactagaaatgacattgccgcatcccttagacaagccatggtattggaggaaggaaagaagatcagaaataatacaagagaagctgctgcaattgtaggaaacttgaagctgcaccaggatcactacgtagctgcatttgttcagtttcttaagaatggaatctggaaaccaatatga

SEQ ID NO: 114 - protein, 461 aa, Glycine max
MAENPIRVTMIPWSAFGHLIPFFKLSIALAKAGVHVSFISTPKNIQRLPKIPSTLSHLVHFVELPLPSLDNDILPEGAEATLDIPFEKHEYLKAAYDKLQDAVKQFVANQLPDWIICDFNPHWVVDIAQEFQVKLILFVIISATGATFIGPPGTRTGPLSPESLTAPPEWVTFPSSVAFRKHEAIHFCAGSYKVSSSGVSDFERIIKLHGASKAVLFRSCYEIEGEYLNAFQKLVEKPVIPIGLLPVERQVVDGCSDTIFEWLDKQASKSVVFVGFGSELKLSKDQVFEIAYGLEESQLPFLWALRKPSWESNDEYSLPVGFIERTSNRGSVCKGWIPQLEILAHSSIGGSLFHSGLGSVIENLQFGHTLVVLPFNIDQPLIARFLVEKGLAIEVKRNEDGSFTRNDIAASLRQAMVLEEGKKIRNNTREAAAIVGNLKLHQDHYVAAFVQFLKNGIWKPI

SEQ ID NO: 115 - DNA, 1386 bp, Glycine max
atggctgagaatccgattcgtgtaacgatgattccatggtctgcctttggccacttgataccatttttcaaactttccatagccttagccaaagctggtgttcatgtctccttcatatccactcccaaaaacattcaaaggcttcccaaaattccttcgactttatctcatttggttcattttgtggaacttcctttaccatcattggacaatgatatcttgccagaaggtgctgaggccaccgtggacattccatttgaaaaacatgagtacttaaaggcagcattagataagctccaagatgctgtgaagcaatttgtggccaatcagttaccagattggataatctgtgatttcaatcctcactgggttgtagacattgcccaagagtttcaggtaaagttaatcttattttctattttatctgctactggaacaacattcatagtaccaccaggtacaagggcaggtcacttatctccagaaagtctaacagctccaccagaatgggtgacatttccatcttcagtggcttttcgaatacacgaggcaatacatttctgtgctggtttcgacaaggtaaattcttccggggtaagtgattttgaaagggttataaagatacatgatgcctctaaagctgtgatatttcgtagctgctacgagattgaaggagaatatctgaatgcgtatcagaaactatttgagaagcctatgattccaattggtttattgcctgtagagagaggagttgttgatgggtgtagtgataacatatttgagtggcttgataaacaagcatcaaaatcagttgtgtttgtggggtttggcagtgagttaaagctgagcaaggatcaagtttttgagatagcttatggacttgaggaatctcaattgccgtttttatgggcactgagaaaaccaagttgggaaagcaatgatgggtattctttacctgttggttttattgaaaggacatctaaccgaggaagagtttgtaagggatggataccacaactagaaatattggcacattcatctattggaggatctttgtttcactctggctggggttctgtcattgaaaaccttcagtttgggaatacacttgttttattaccctttaatatagagcaacctcttaatgcaaggtttttggtggaaaagagattagccattgaagtgaaaagaaatgaagatggatcattcactagaaatgacattgccgcatcccttagacaagccatggtattggaggaaggaaagaagatcagaaataatacaagagaagctgctgcaattgtaggaaacttgaagctgcaccaggatcactacgtagctgcatttgtacagtttcttaagaatggaatcaggaaaccaatatga

SEQ ID NO: 116 - protein, 461 aa, Glycine max
MAENPIRVTMIPWSAFGHLIPFFKLSIALAKAGVHVSFISTPKNIQRLPKIPSTLSHLVHFVELPLPSLDNDILPEGAEATVDIPFEKHEYLKAALDKLQDAVKQFVANQLPDWIICDFNPHWVVDIAQEFQVKLILFSILSATGTTFIVPPGTRAGHLSPESLTAPPEWVTFPSSVAFRIHEAIHFCAGFDKVNSSGVSDFERVIKIHDASKAVIFRSCYEIEGEYLNAYQKLFEKPMIPIGLLPVERGVVDGCSDNIFEWLDKQASKSVVFVGFGSELKLSKDQVFEIAYGLEESQLPFLWALRKPSWESNDGYSLPVGFIERTSNRGRVCKGWIPQLEILAHSSIGGSLFHSGWGSVIENLQFGNTLVLLPFNIEQPLNARFLVEKRLAIEVKRNEDGSFTRNDIAASLRQAMVLEEGKKIRNNTREAAAIVGNLKLHQDHYVAAFVQFLKNGIRKPI

SEQ ID NO: 117 - DNA, 1077 bp, Glycine max
atggcttcttcattaaacaatggccgtaaagcaagtgagatttttcaaggtcaagctctcttgtacaaacatttgcttggcttcatagattctaagtgtctaaaatggatggttgagcttgacatacccgacataatccacagccatagccatggccaacccattactttttcagagttggtgtcaattctacaagtcccaccaactaaaactcgtcaggtgcagagcctcatgcgttatctagcacacaatggattctttgagatagtgagaatccatgacaacatagaagcatatgctctcactgctgcttcagagttacttgtcaaaagcagtgagcttagtttagctccaatggttgagtattttcttgaaccaaattgtcaaggtgcatggaaccagttgaagaggtgggttcatgaggaagatctcacagtatttgaggtctccttaggaacacctttctgggactttatcaataaagaccctgcatataacaagtcattcaatgaggcaatggcttgtgattctcagatgttgaacttggcgtttagagattgcaattgggtctttgagggactggaatccattgtggatgttggtggtggaactggaatcacagcaaagattatctgtgaggcttttcctaagctgaaatgcatggtgttggaacgtccaaatgttgtggaaaatttgtcaggaagcaacaatttgacatttgttggtggggacatgtttaaatgcatccccaaggctgatgcagttctgcttaagttggttttacataattggaatgacaacgattgcatgaagatattagaaaattgtaaagaagctatttcaggtgaaagcaaaacaggaaaagtagttgtcatagatactgtgataaacgaaaacaaagatgagcgccaagttactgaactaaagctccttatggatgtacacatggcatgtattattaatggaaaagagagaaaagaagaagattggaagaaactcttcatggaagcagggttccaaagctacaaaatatctcccttcacaggatatttgtctcttattgagatctatccttga

SEQ ID NO: 118 - protein, 358 aa, Glycine max
MASSLNNGRKASEIFQGQALLYKHLLGFIDSKCLKWMVELDIPDIIHSHSHGQPITFSELVSILQVPPTKTRQVQSLMRYLAHNGFFEIVRIHDNIEAYALTAASELLVKSSELSLAPMVEYFLEPNCQGAWNQLKRWVHEEDLTVFEVSLGTPFWDFINKDPAYNKSFNEAMACDSQMLNLAFRDCNWVFEGLESIVDVGGGTGITAKIICEAFPKLKCMVLERPNVVENLSGSNNLTFVGGDMFKCIPKADAVLLKLVLHNWNDNDCMKILENCKEAISGESKTGKVVVIDTVINENKDERQVTELKLLMDVHMACIINGKERKEEDWKKLFMEAGFQSYKISPFTGYLSLIEIYP

SEQ ID NO: 119 - DNA, 1062 bp, Glycine max
  misc_feature (615)..(615): n is a, c, g, or t
atggcttcttcaatcaatggccgtaatgaaagtgagatctttcaaggtcaaactctcttgtacaaacacttgtatgccttcatagactctatgtgtctcaaatgcattgttgagcttggaataccagacataatccacaaccatggccaacccattactcttcctgagttggtgtcaattctacaaattccaccagctaaagttagtcaagtgcaaagtctcatgcgctacctggaacacaatggattctttgaaagagtaagaatccacgaaaaagaagcatatgctctcactgctgcttcagagctacttgtcaaaagcagtgagcttagtttagctccaatgattgagtttgttcttgacccaactttgtcaaattcattccatcaattgaaaaagtgggtttatgagaaagatctcacattatttgacatctccttaggatcacatttgtgggactttcttaataaaaaccctgcacataacaagtcattcaatgaggcaatggctagtgattctcagatgatgaacttggcgttgagagattgcaattgggtctttcaaggactggaattcattgtggatgttggtggtggaactggaaccacagccaanattatatgtgaagcatttcctaacttgaaatgcattgtgtttgaccgtccacaagttatagagaacttgtcaggaagcaacaatttgacatatgttggtggggacatgttcaaatctattcctaaggctgatgtaattctgcttaagtggattctacataattggattgataaggattgcataaagatattgaagaactgcaaagaagctatttcaaataatggcaaaagaggaaaagtgattatcatagatgttgtgataaacgaaaaagaagatgagcacaaagttacggaactaaagctcgttatggatataaccatggcatgtgttaatggaaaagagagaaatgaagaagaatggaagaaactcttcatggaagcagggttccaagactacaaaatatttcccttgaccaaatatttgtctgttattgagatctatccttag

SEQ ID NO: 120 - protein, 353 aa, Glycine max
  misc_feature (205)..(205): Xaa can be any naturally occurring amino acid
MASSINGRNESEIFQGQTLLYKHLYAFIDSMCLKCIVELGIPDIIHNHGQPITLPELVSILQIPPAKVSQVQSLMRYLEHNGFFERVRIHEKEAYALTAASELLVKSSELSLAPMIEFVLDPTLSNSFHQLKKWVYEKDLTLFDISLGSHLWDFLNKNPAHNKSFNEAMASDSQMMNLALRDCNWVFQGLEFIVDVGGGTGTTAXIICEAFPNLKCIVFDRPQVIENLSGSNNLTYVGGDMFKSIPKADVILLKWILHNWIDKDCIKILKNCKEAISNNGKRGKVIIIDVVINEKEDEHKVTELKLVMDITMACVNGKERNEEEWKKLFMEAGFQDYKIFPLTKYLSVIEIYP

SEQ ID NO: 121 - DNA, 1032 bp, Medicago sativa
atggcttcatcaattaatggccgaaaaccaagtgaaattttcaaagcacaagctttattatacaaacatatatatgccttcatagattccatgtctcttaaatgggctgttgaaatgaacataccaaacataatccaaaaccatggcaaaccaatttctctttcaaacttagtttcaattcttcaagttccatcgtcgaaaataggtaacgtgcggcgtctcatgcgttacctcgcgcacaacggattcttcgagataattacaaaagaagaagagtcttatgctctcactgttgcttcagagcttcttgttagaggcagtgatctttgtttagcaccaatggttgagtgtgttcttgatccaactctttcgggttcgtatcatgagctgaagaaatggatttatgaggaagatcttacactctttggtgttactttaggatctggtttttgggattttcttgataaaaatcctgaatataatacctcatttaatgatgcaatggctagtgattctaaattgataaacttggcattgagagattgtgattttgtgtttgatggattggaatcaattgtggatgttggtggtggaactggaacaactgctaagattatttgtgagacttttcctaagttgaaatgtattgtgtttgataggccacaagttgtagagaacttatctggaagcaataatttgacttatgttggtggggacatgttcacatctattcctaatgctgatgcagttttgcttaaggattgcctaaggatactgaagaaatgtaaagaagctgttacaaatgatgggaaaagaggaaaagtgactattatagacatggtgatagatgaaaaaaaagatgagaatcaagttactcaaattaagctccttatggatgtaaacatggcttgtctaaatggaaaagagagaaatgaggaagaatggaagaaactcttcatagaagctggtttccaacactataagatatctcctttgactggatttttgtctcttattgagatctatccataa

SEQ ID NO: 122 - protein, 343 aa, Medicago sativa
MASSINGRKPSEIFKAQALLYKHIYAFIDSMSLKWAVEMNIPNIIQNHGKPISLSNLVSILQVPSSKIGNVRRLMRYLAHNGFFEIITKEEESYALTVASELLVRGSDLCLAPMVECVLDPTLSGSYHELKKWIYEEDLTLFGVTLGSGFWDFLDKNPEYNTSFNDAMASDSKLINLALRDCDFVFDGLESIVDVGGGTGTTAKIICETFPKLKCIVFDRPQVVENLSGSNNLTYVGGDMFTSIPNADAVLLKDCLRILKKCKEAVTNDGKRGKVTIIDMVIDEKKDENQVTQIKLLMDVNMACLNGKERNEEEWKKLFIEAGFQHYKISPLTGFLSLIEIYP

SEQ ID NO: 123 - DNA, 1059 bp, Medicago truncatula
atggcttcatcaattaatggccgaaaaccaagtgaaattttcaaagcacaagctttattatacaaacatatatatgccttcatagattccatgtctcttaaatgggctgttgaaatgaacataccaaacataatccaaaaccatggcaaaccaatttctctttcaaacttagtttcaattcttcaagttccatcgtcgaaaataggtaacgtgcggcgtctcatgcgttacctcgcgcacaacggattcttcgagataattacaaaagaagaagagtcttatgctctcactgttgcttcagagcttcttgttagaggcagtgatctttgtttagcaccaatggttgagtgtgttcttgatccaactctttcgggttcgtatcatgagctgaagaaatggatttatgaggaagatcttacactctttggtgttactttaggatctggtttttgggattttcttgataaaaatcctgaatataatacctcatttaatgatgcaatggctagtgattctaaattgataaacttggcattgagagattgtgattttgtgtttgatggattggaatcaattgtggatgttggtggtggaactggaacaactgctaagattatttgtgagacttttcctaagttgaaatgtattgtgtttgataggccacaagttgtagagaacttatctggaagcaataatttgacttatgttggtggggacatgttcacatctattcctaatgctgatgcagttttgcttaagtatattctacataattggactgataaggattgcctaaggatactgaagaaatgtaaagaagctgttacaaatgatgggaaaagaggaaaagtgactattatagacatggtgatagatgaaaaaaaagatgagaatcaagttactcaaattaagctccttatggatgtaaacatggcttgtctaaatggaaaagagagaaatgaggaagaatggaagaaactcttcatagaagctggtttccaacactataagatatctcctttgactggatttttgtctcttattgagatctatccataa

SEQ ID NO: 124 - protein, 352 aa, Medicago truncatula
MASSINGRKPSEIFKAQALLYKHIYAFIDSMSLKWAVEMNIPNIIQNHGKPISLSNLVSILQVPSSKIGNVRRLMRYLAHNGFFEIITKEEESYALTVASELLVRGSDLCLAPMVECVLDPTLSGSYHELKKWIYEEDLTLFGVTLGSGFWDFLDKNPEYNTSFNDAMASDSKLINLALRDCDFVFDGLESIVDVGGGTGTTAKIICETFPKLKCIVFDRPQVVENLSGSNNLTYVGGDMFTSIPNADAVLLKYILHNWTDKDCLRILKKCKEAVTNDGKRGKVTIIDMVIDEKKDENQVTQIKLLMDVNMACLNGKERNEEEWKKLFIEAGFQHYKISPLTGFLSLIEIYP

SEQ ID NO: 125 - DNA, 1059 bp, Medicago truncatula
atggcatcatcacttaatggcagaaaaccaagtgaaattttcaaggcacaagctttattatacaaacacatatttgccttcatagattccatgtctcttaaatgggctgttgaaatgaacataccaaatattattcacaatcatggcaaaccaatttcactttcaaatttagtttcaattcttcaagttccatcgtcgaaaataggtaacgtgcggcgtctcatgcgttacctcgcgcacaacggattctttgagataattacaaaagaagaagagtcttatgctctcactgttgcttcagagcttcttgttagaggtagtgatctttgtttagcaccaatggttgagtgtgttcttgatccaactctttcgggttcgtatcatgagctgaagaaatggatttatgaggaagatcttacactctttggtgttactttagggtctggtttttgggattttcttgataaaaatcctgaatataatagatcatttaatgatgcaatggctagtgattctaaattgataaacttggcattgagagattgtgattttgtgtttgatggattggaatcaattgtggatgttggtggtggaaatggaaccactggtaaaattatttgtgagacttttcctaagttgaaatgtattgtgtttgataggccacaagttgtagagaacttatctggaagcaataatttgacttatgttggtggggacatgttcacatctattcctaatgctgatgcagttttgcttaagtatattttacataattggactgataaggattgcacaaggatactgaagaaatgtaaagaagctgttacaaatgatgggaaaaaagggaaggtgattattatagatatggtcataaatgaaaagaaagatgagaatcaagttactcaaattaagctccttatggatgtaaacatggcttgtcttaatggaaaagagagaaatgaggaagaatggaagaaactcttcatagaagctggtttccaagactataagatatctcctttgactggatttttgtctcttattgagatctatccataa

SEQ ID NO: 126 - protein, 352 aa, Medicago truncatula
MASSLNGRKPSEIFKAQALLYKHIFAFIDSMSLKWAVEMNIPNIIHNHGKPISLSNLVSILQVPSSKIGNVRRLMRYLAHNGFFEIITKEEESYALTVASELLVRGSDLCLAPMVECVLDPTLSGSYHELKKWIYEEDLTLFGVTLGSGFWDFLDKNPEYNRSFNDAMASDSKLINLALRDCDFVFDGLESIVDVGGGNGTTGKIICETFPKLKCIVFDRPQVVENLSGSNNLTYVGGDMFTSIPNADAVLLKYILHNWTDKDCTRILKKCKEAVTNDGKKGKVIIIDMVINEKKDENQVTQIKLLMDVNMACLNGKERNEEEWKKLFIEAGFQDYKISPLTGFLSLIEIYP

SEQ ID NO: 127 - DNA, 759 bp, Glycine max
atggaggttggcctcaagaaggagaacatggggtttgaggaaactgagttaaggcttggactgcctggaaacggaggcactgaagaagtgctcatcaggaagaggggtttctctgagactgaaactggtcatgaagatgagtctgccaccactgtggatttgatgcttaatctttcttctaaggaggccgcaaccactgctgctgctgctgcagatccaactgataagcacaagactttgcctaaggagaagacccttctgccagcagatcctgctaagcctccagccaagacgcaggtggtgggttggccacctgtgcggtccttccggaagaacatgttggctgtgcaaaagagcgttggagaagagagcgagaagaacagcagccctaatgcaagctttgtcaaagttagcatggatggagcaccttaccttcgcaaagtggacttgaagatgtacaagagttaccgagagctctctgattctttaggcaaaatgttcagctccttcacctttggcaattgtgaatcccaaggaatgaaggatttcatgaatgagagcaagctgaatgatctcttgaacagctctgattatgtcccaacctatgaggacaaggatggtgactggatgcttgtcggtgatgtcccatgggagatgtttgttgaatcatgcaagcgtttacgcatcatgaaaggaaaggaggctattggtcttggtcttgcaccaagagccatggcaaaatgcaagaacaggagctag

SEQ ID NO: 128 - protein, 243 aa, Glycine max
MGFEETELRLGLPGNGGTEEVLIRKRGFSETETGHEDESATTVDLMLNLSSKEAATTAAAAADPTDKHKTLPKEKTLLPADPAKPPAKTQVVGWPPVRSFRKNMLAVQKSVGEESEKNSSPNASFVKVSMDGAPYLRKVDLKMYKSYRELSDSLGKMFSSFTFGNCESQGMKDFMNESKLNDLLNSSDYVPTYEDKDGDWMLVGDVPWEMFVESCKRLRIMKGKEAIGLGLAPRAMAKCKNRS

SEQ ID NO: 129 - DNA, 747 bp, Glycine max
atggtgtttgaggaaactgagctgaggcttggactgagactaggactccctggaaatggagccgcgccaacaactgaagctgctgcggaattaggagtgaggaagagagggttctctgaaactgaaaccgatgaaacaacctccgttgatttgatgcttaacctctctcccaaggaagcttctgctgctgctactactgatggagcagatccacgtgagaatccaaagacttcgccgaaggagaagaatcttcctcttctcgatcccgcaaagcctcctgccaaggcgcaagtggtgggatggccacctgtaaggtctttccggaagaacatgttcgcagcccaaaagagcagcggcggagaggaaagcgaaaagagcagccctaatgcaagctttgtcaaagttagcatggatggagcaccttacctccgcaaagttgacttgaagatgtacaagagttacccagagctctctgatgccttgggcaaaatgtttagctccttcaccattggaaattgtgaatcccaaggcttcaaggatttcatgaatgagagcaagttgatggatcttttgaacagctccgactatgtcccgacctatgaagacagggatggcgactggatgcttgtcggtgatgtgccatgggagatgtttgttgaatcatgcaagcgtttacgtatcatgaaaggaaaggaagctattggactggcaccaagagccgtggagaaatgcaagaacaggagctag

SEQ ID NO: 130 - protein, 248 aa, Glycine max
MVFEETELRLGLRLGLPGNGAAPTTEAAAELGVRKRGFSETETDETTSVDLMLNLSPKEASAAATTDGADPRENPKTSPKEKNLPLLDPAKPPAKAQVVGWPPVRSFRKNMFAAQKSSGGEESEKSSPNASFVKVSMDGAPYLRKVDLKMYKSYPELSDALGKMFSSFTIGNCESQGFKDFMNESKLMDLLNSSDYVPTYEDRDGDWMLVGDVPWEMFVESCKRLRIMKGKEAIGLAPRAVEKCKNRS

SEQ ID NO: 131 - DNA, 747 bp, Trifolium pratense
atggaagttgcagctggggtgatgaaaaaggagaagatggtgtttgatgaaactgaactgcggttgggattgcctggaaatattggagctggaaaagtaacagaagtagttgcaaggaagagaggtttctctgaaactgagagtgaaagtgatcaaacaaacactgtggatttgaagcttaatctctcttccaaagaaggagcagcagatccaattgagaagactaaggagaagactcttctgctctctgattctggcgccaagccacctgctaaggcgcaagtggtcggctggccaccagtgcggtcttaccggaagaacatgtttgcagctcaaaagagcaacaaaggattagaggaaagtgagaagaccaccactaatccaataagctttgtgaaagttagcatggatggtgcaccttacctccgcaaagtagatctcaagatgtacaagagttacccagagctctctgatgccttggccaaaatgtttaacctctttaccaacggaaattgtgaatcccaaggcactaaggatttcatgaaggagagtaataaggtgatggatctattgaacagctccgactatgtcccaacttatgaagacaaggatggtgactggatgcttgtcggtgatgtgccatgggagatgttcattgaatcatgcaagcgtttacgtatcatgaaaggaaaagaagctgttggactcgcaccaagagctatggaaaaatgcaagaacgggagctag

SEQ ID NO: 132 - protein, 248 aa, Trifolium pratense
MEVAAGVMKKEKMVFDETELRLGLPGNIGAGKVTEVVARKRGFSETESESDQTNTVDLKLNLSSKEGAADPIEKTKEKTLLLSDSGAKPPAKAQVVGWPPVRSYRKNMFAAQKSNKGLEESEKTTTNPISFVKVSMDGAPYLRKVDLKMYKSYPELSDALAKMFNLFTNGNCESQGTKDFMKESNKVMDLLNSSDYVPTYEDKDGDWMLVGDVPWEMFIESCKRLRIMKGKEAVGLAPRAMEKCKNGS

SEQ ID NO: 133 - DNA, 753 bp, Unknown
  Synthetic sequence
atggaagttgttggtatgaagaaggagaatatgggatttgaggaaactgagttgagacttggaattggatttcttggaaataatggatcagccacagctactgaaggagttgtaaggaagagaggattttctgagactgaaactgatgatgatacaactactatggatttgatgcttaatctttcctctaaggaagctactgctgaggtagatccaagtgatataaccaccaagactttgcaaaaggagaagacccttttgcctgcagatcctgccaagcctcctgcaaaggctcaagtggtgggttggccacctgtccggtcgtaccggaagaacatgttagcaatgcaaaagagtgaaagtgagaagaacagcagttccaatttcaatgcaattacatttgtgaaagttagtatggatggagctccttaccttcgtaaggttgacttgaagatgtacacaagttactcacagctttctgattccttaggcaaaatgttcagctccttcaccattggcaactgtgaatctcaaggaatgaaggatttcatgaatgagagtaagttgatggatcttttaaacaattctgattatgttccaacctatgaagacaaggatggtgactggatgcttgtcggtgacgtcccatgggagatgtttgttgaatcatgcaaacgtttgcgtatcatgaaaggaaaggaggctattggtatagcaccaagagctatggaaaaatgcaagaacaggagctag

SEQ ID NO: 134 - protein, 250 aa, Unknown
  synthetic sequence
MEVVGMKKENMGFEETELRLGIGFLGNNGSATATEGVVRKRGFSETETDDDTTTMDLMLNLSSKEATAEVDPSDITTKTLQKEKTLLPADPAKPPAKAQVVGWPPVRSYRKNMLAMQKSESEKNSSSNFNAITFVKVSMDGAPYLRKVDLKMYTSYSQLSDSLGKMFSSFTIGNCESQGMKDFMNESKLMDLLNNSDYVPTYEDKDGDWMLVGDVPWEMFVESCKRLRIMKGKEAIGIAPRAMEKCKNRS

SEQ ID NO: 135 - DNA, 1999 bp, Arabidopsis thaliana
gtagtgcccttcatggataccaaaagagaaaatttgatttagtgcatacatataacaatataacgccgcataataatactgtataaaacagtcatgtaacgatatgacagcagtaatacagttccaagagacgttataatcgtatgcaatcatatgcttgcgtagattttccaacagttttgtttcgttgataggaggaactcaacactctagggtagtgattggtagacactattagcacaaaaaatattaattttactctgatgtttaccaaaaaagttaccaatcaaatatttaagagatcgtactcttccacggcgactctaaaaaccaaagatataggttagactcataactactttataaagaaaatgtttaacgataactaccgagatctaataaataaaccttcattttcaagtatattatatttgcttcttttgtttatatatcaaaccaagttctggtttataaaaatattagataaaactcgtctaaataggtaggtgtaaaataaaattttaaatttttatcgataatatttaaaatttgaaaagttaataatgatccacacattttttctaatatttaatttagtaatttttgtattaaataaaatttcaatcatatacattcgatttttctatacattttaactatctatttctgcataataaactgtattttcattttatacgcttcatcttatggatgatatttaaattttaaatagtaattcatacactttttaatatttaatttagtattttcttaaatccaaattttaatcttacaatttaaatatctactttaacataatacaaatacaatttaatttcattgtattaaattcaaatataatttgattataataaaatacaatttaattctaaaaagtccatcttagattttaattttcctttttagttttgaaaattaaaaatttaaatttattagatatatatgttactttttcagttttcctatttatttaagaaaaaaatattttttaacacatgtcaacttgtaaacaatagactgaacacgtcattttatattatgtttagttttgaaaattaaagttaattaaatatttatatttcttttttttagcttttctaattatttttaaaatagtaaatatttttaatacaaatcaatatctgaacaatagatttgatacataacataatcctataaattattaacttggaaaacgatagtttatataataaaattattttcttaagttctctaaccataacaattaaactatattttagcgaagaaaagaagagaataccgagagaacgcaacttgcactaaaagctaccactttggcaaatcactcatttatattattatatactatcacctcaattcaatcgaaacctcaaaataacactaatatatacacaaagaaacaacagaataacaccgaagaatataggtttaggaaaatccagaatttgttgagactaaagagatcaaattttcgatacaaggttttgctcaatttgtattttcataataaaattctttatttcaccatagacttacatgattagtttttcttttaataaaaaaaaacacgcgacatgaaaattatattatctcagtgttgtcgaatttgaatttgaattttgagttaaatactacacatttgttgacaacttattaaactttacaagtctgctacaaatattgtcaaatatttactaattaatggaccaaaatcctctaacttgcaaatttgtatctacatcaacttaaaaattaggaatatgcgacccaaaaaaaaaaaaactaggaataataataaaaaaatggaatgatgtggaggaagctctttactctttgagaggaagtttataaattgaccacacatttagtctattatcatcacatgtattaagacttgacaacttgtctttctcacaccaaacccctctcctctgtttcataacatctgctctttcttttttttcctaagccccta

SEQ ID NO: 136 - DNA, 609 bp, Glycine max
gaagccacgtcatgaagagtatatcatttcagtaatgttttgagacgcctctataatgctttaccaacaaaacaaaacaaaaaaaagaacatttgaaaccatttgtattaaaaaaaaaaaggtatattaggccataatattataggtaacatgaaatatcaaatgacacgcaagagttttgtcaaaaatgaaaccatcacacatcagagattatggcaaataatgttttgtgtgtctcttgcttcacccataacataagcctctataactggagagaagaaaaaaaaaagtggaggggctagggtgggaatttggaagaatacagttatattgagcattgagcaagttgatagaaagcttctcaatttgtacaaaatttgcatccacatgattattaaagacgtagacagcacttcttccttctttttttctataagtttcttatatattgttcttcatgttttaatattattactttatgtacgcgtctaacagtagtcctcccaaactgctataaatagagcctcttcaacgcacctcttggcagtacaaaaattattcatctcttctaagttctaattttctaagcattcagtaaaagaactaacc

SEQ ID NO: 137 - DNA, 325 bp, Petroselinum crispum
aattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctc

SEQ ID NO: 138 - DNA, 1112 bp, unknown
  Synthetic sequence
ggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccgcgagctggtcaatcccattgcttttgaagcagctcaacattgatctctttctcgatcgagggagatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagtcaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcga